(19) United States
(12) Patent Application Publication
(10) Pub. No.: US 2026/0112487 A1
(43) Pub. Date: Apr. 23, 2026

(54) SYSTEMS AND METHODS TO MEASURE, PREDICT AND OPTIMIZE BRAIN FUNCTION

(71) Applicant: Horizon Neurosciences LLC, Pittsfield, MA (US)

(72) Inventor: Emiliano Santarnecchi, Pittsfield, MA (US)

(21) Appl. No.: 18/871,615

(22) PCT Filed: Jun. 5, 2023
(86) PCT No.: PCT/US23/24442

§ 371 Date: Dec. 4, 2024

Related U.S. Application Data
(60) Provisional application No. 63/349,382, filed on Jun. 6, 2022.
(60) Provisional application No. 63/354,469, filed on Jun. 22, 2022.

(51) Int. Cl. G16H 50/20; A61B 5/372

(52) U.S. Cl. CPC G16H 50/20; A61B 5/372

(57) ABSTRACT

Methods and apparatus for changing a brain state of a person from an initial brain state to a target brain state are described. The method includes receiving information characterizing the initial brain state, the information including a structural composition and a functional architecture of the brain, estimating based, at least in part, on the received information, a potential for the brain to change from the initial brain state to the target brain state, determining based, at least in part, on the received information and the estimated potential for the brain to change from the initial brain state to the target brain state, a non-invasive brain stimulation protocol, and controlling at least one non-invasive brain stimulation device to stimulate the brain according to the non-invasive brain stimulation protocol. The method also includes using brain information to inform the design of computational general artificial intelligence agents.

### FIELD OF INVENTION

The disclosure relates to systems and methods for measuring, predicting, and optimizing brain function in humans.

### BACKGROUND

The brain is a complex dynamic system characterized by topographic organization in distributed neural networks with specific temporal and spatial properties. Each individual brain is different, in terms of its structural and functional properties, and only a partial understanding of how brain activity explains cognition and behavior is available today. Moreover, very limited knowledge is available on how the brain adapts over time or in response to external and internal perturbations, as in the case of normal healthy aging, a trauma, dementia, brain cancer, or even exposure to a cognitive training aimed at improving abilities such as memory or attention. Additionally, models able to simulate and predict such changes are generally not currently available, and those developed so far not adopting artificial intelligence algorithms that take into account principles of brain functioning.

### SUMMARY

Some embodiments of the present disclosure relate to techniques for mapping, characterizing, predicting and/or optimizing brain function. Systems and methods described herein include data analysis and visualization tools, algorithms for estimation of brain potential and corresponding strategies for brain, cognitive and behavioral enhancement, hardware for data collection and neuromodulation, and application-specific algorithms for the generation of digital assets based on individual brain activity features.

In some embodiments, a method of changing a state of a brain of a person from an initial brain state to a target brain state is provided. The method includes receiving information characterizing the initial brain state, the information including a structural composition and a functional architecture of the brain of the person, wherein the information includes passive and active data recorded from the brain of the person, estimating based, at least in part, on the received information characterizing the initial brain state, a potential for the brain of the person to change from the initial brain state to the target brain state, determining based, at least in part, on the received information characterizing the initial brain state and the estimated potential for the brain of the person to change from the initial brain state to the target brain state, a non-invasive brain stimulation protocol, and controlling at least one non-invasive brain stimulation device to stimulate the brain of the person according to the non-invasive brain stimulation protocol to change the state of the brain of the person from the initial brain state to the target brain state.

In one aspect, estimating a potential for the brain of the person to change from the initial brain state to the target brain state comprises using an algorithm to analyze the received information characterizing the initial brain state. In another aspect, the algorithm is configured to enhance cognition and brain health. In another aspect, the algorithm is configured to optimize an intervention aimed at treating neurological or psychiatric conditions. In another aspect, the method further includes extracting at least one metric from the algorithm, and storing the at least one metric as a non-fungible token. In another aspect, the method further includes using the non-fungible token to perform one or more of tracking progress of an intervention associated with the non-invasive brain stimulation protocol, defining brain and cognitive stimulation approaches, or comparing a dynamic multilayer digital twin (DMDT) associated with the person to a population-level DMDT to compute a distance matrix.

In another aspect, the method further includes extracting at least one metric from the algorithm, and using the extracted at least one metric to inform a neuromorphic AI platform. In another aspect, the method further includes extracting at least one metric from the algorithm, and using the extracted at least one metric to set parameters for the noninvasive brain stimulation protocol. In another aspect, the method further includes extracting at least one metric from the algorithm, and using the extracted at least one metric to create avatars and content for a gaming and/or metaverse application. In another aspect, the method further includes extracting at least one metric from the algorithm, and using the extracted at least one metric to define personalized learning trajectories for skill acquisition, wherein the potential for the brain of the person to change from the initial brain state to the target brain state is estimated based, at least in part, on the personalized learning trajectories for skill acquisition. In another aspect, the method further includes extracting at least one metric from the algorithm, and using the extracted at least one metric to derive measures of brain plasticity and resilience, wherein the potential for the brain of the person to change from the initial brain state to the target brain state is estimated based, at least in part, on the measure of brain plasticity and resilience. In another aspect, the method further includes extracting at least one metric from the algorithm, and using the extracted at least one metric to derive interventions aimed at increasing one or more of brain resilience, brain plasticity, or overall brain health, wherein the non-invasive brain stimulation protocol is determined based, at least in part, on the derived interventions aimed at increasing one or more of brain resilience, brain plasticity, or overall brain health.

In another aspect, the method further includes extracting at least one metric from the algorithm, and using the extracted at least one metric to estimate metrics of brain evolution potential and/or state/trait transition, wherein the potential for the brain of the person to change from the initial brain state to the target brain state is estimated based, at least in part, on the estimated metrics of brain evolution potential and/or state/trait transition. In another aspect, the method further includes extracting at least one metric from the algorithm, and using the extracted at least one metric to estimate specific brain states related to one or more of thinking about the future, the past, emotional content, or specific memories, wherein the potential for the brain of the person to change from the initial brain state to the target brain state is estimated based, at least in part, on the estimate specific brain states related to one or more of thinking about the future, the past, emotional content, or specific memories. In another aspect, the method further includes extracting at least one metric from the algorithm, and using the extracted at least one metric to create templates of specific brain states related to one or more of thinking about the future, the past, emotional content, specific memories, wherein the potential for the brain of the person to change from the initial brain state to the target brain state is estimated based, at least in part, on the templates of specific brain states related to one or more of thinking about the future, the past, emotional content, specific memories. In another aspect, the method further includes using the specific brain states to classifying data from healthy controls and patients to assess brain health of the healthy controls and patients.

In another aspect, the method further includes extracting at least one metric from the algorithm, and using the extracted at least one metric to estimate disease progression in a neurodegenerative condition, wherein the potential for the brain of the person to change from the initial brain state to the target brain state is estimated based, at least in part, on the estimated progression in a neurodegenerative condition. In another aspect, the method further includes extracting at least one metric from the algorithm, and using the extracted at least one metric to estimate disease progression in patients with brain tumors, wherein the potential for the brain of the person to change from the initial brain state to the target brain state is estimated based, at least in part, on the estimated disease progression in patients with brain tumors. In another aspect, the method further includes extracting at least one metric from the algorithm, and using the extracted at least one metric to estimate disease progression and symptoms in patients with depression, wherein the potential for the brain of the person to change from the initial brain state to the target brain state is estimated based, at least in part, on the estimated disease progression and symptoms in patients with depression.

In another aspect, the method further includes extracting at least one metric from the algorithm, and using the extracted at least one metric to estimate disease progression and symptoms in patients with brain cancer, wherein the potential for the brain of the person to change from the initial brain state to the target brain state is estimated based, at least in part, on the estimated disease progression and symptoms in patients with brain cancer. In another aspect, the target brain state is a brain state predisposing to maximal clinical benefits of brain surgery in patients with brain cancer. In another aspect, the target brain state is a brain state with the least probability of experiencing tumor recurrence in patients with brain cancer.

In another aspect, the method further includes extracting at least one metric from the algorithm, and using the extracted at least one metric to estimate disease progression and symptoms in patients with sleep disorders, wherein the potential for the brain of the person to change from the initial brain state to the target brain state is estimated based, at least in part, on the estimated disease progression and symptoms in patients with sleep disorders. In another aspect, the target brain state is a brain state predisposing to a highest duration of deep sleep stages during sleep in patients with sleep disorders.

In some embodiments, a method of changing a state of a brain of a person to a target brain state is provided. The method includes receiving information associated with digital replica of an initial brain state of the person, the digital replica including a plurality of brain metrics, determining based, at least in part, on the received information associated with the digital replica of the initial brain state of the person, a non-invasive brain stimulation protocol to change the state of the brain of the person from the initial brain state to the target brain state, and stimulating, with at least one non-invasive brain stimulation device, the brain of the person according to the determined non-invasive brain stimulation protocol to change the state of the brain of the person from the initial brain state to the target brain state.

In some embodiments, a method of utilizing brain data to create digital content and architectures used for a videogame or the metaverse is provided. The method includes receiving information characterizing a structural and functional architecture of a brain of a person, wherein the information includes electrophysiological and neuroimaging data recorded from the person, analyzing the received information to create metrics of brain activity and performance in the form a digital twin, and creating first videogame or metaverse content based, at least in part, on the metrics of brain activity and performance in the digital twin.

In one aspect, the received information includes data sensed by a wearable device worn by the person. In another aspect, the method further includes receiving updated information from a network connected device, updating the digital twin based on the received updated information, and creating second videogame or metaverse content based, at least in part, on the updated digital twin. In another aspect, the first videogame or metaverse content comprises a multilayer avatar. In another aspect, the first videogame or metaverse content includes in-game mechanics. In another aspect, the in-game mechanics include in-game physics and/or an avatar leveling system. In another aspect, the first videogame or metaverse content comprises a unique digital asset for the person. In another aspect, the unique digital asset for the person comprises equipment in a videogame. In another aspect, the first videogame or metaverse content comprises artificial intelligence used to control one or more non-playing character in a videogame. In another aspect, the first videogame or metaverse content comprises at least one in-game progression sequence or storyline. In another aspect, the first videogame or metaverse content comprises a clinical metaverse application for patients with neurological and psychiatric conditions.

In some embodiments, a method of creating a digital replica of a brain state of a person is provided. The method includes receiving information associated with a brain of the person, the information including structural brain information and functional brain information, the functional brain information including electrophysiological data recorded from the person, processing the received information to determine a plurality of brain metrics that characterize a brain state of the person, and creating a digital replica of the brain state of the person based, at least in part, on the plurality of brain metrics determined from received information.

In one aspect, the method further includes designing a neuromorphic artificial intelligence agent based on the digital replica of the brain state of the person. In another aspect, the method further includes updating the digital replica of the brain state of the person based, at least in part, on an updated plurality of brain metrics determined from updated information associated with the brain of the person, and updating the neuromorphic artificial intelligence agent based on the updated digital replica of the brain state of the person. In another aspect, the neuromorphic artificial intelligence agent is based on oscillatory generators reflecting the brain state and a brain architecture of the person. In another aspect, the neuromorphic artificial intelligence agent is used as a conversational agent in a therapeutic setting. In another aspect, the neuromorphic artificial intelligence agent is used to increase the efficacy of a mental health intervention, the mental health intervention including a cognitive behavioral therapy.

In some embodiments, an apparatus for modifying a brain state of a person is provided. The apparatus includes at least one sensor configured to sense brain activity signals from a brain of the person, a stimulation device configured to provide non-invasive stimulation to the brain of the person, and a controller configured to control operation of the stimulation device based, at least in part, on one or more characteristics of the brain activity signals sensed from the at least sensor following stimulation of the brain of the person with the stimulation device.

In some embodiments, a method of providing personalized modulation of brain activity to a person to alter a brain state of a brain of the person is provided. The method includes sensing, in response to providing non-invasive stimulation to the brain of the person, a plurality of evoked potentials using a plurality of electrophysiological sensors, determining based, at least in part, on at least one characteristic of the plurality of evoked potentials, a personalized modulation plan for the person, the personalized modulation plan including a location of stimulation and one or more stimulation characteristics, and stimulating the brain of the person according to the personalized modulation plan to alter a brain state of the person.

In some embodiments, a method of personalizing a digital experience within a digitally-created environment is provided. The method includes receiving information associated with digital replica of a brain of a user, the digital replica including a plurality of brain metrics for the user, configuring at least one aspect of the digitally-created environment based, at least in part, on the plurality of brain metrics for the user included in the digital replica to create a personalized digitally-created environment for the user, and displaying the personalized digitally-created environment to the user.

In some embodiments, a method of inducing plasticity of a brain of a person is provided. The method includes receiving information associated with digital replica of a brain of a person, the digital replica including a plurality of brain metrics for the person, the plurality of brain metrics including a first plasticity level of the brain of the person, controlling at least one non-invasive brain stimulation device to deliver non-invasive stimulation to at least one location in the brain of the person, the at least one location having been identified as a location to induce plasticity of the brain of the person, and determining a second plasticity level of the brain of the person based, at least in part, on feedback received following stimulation of the at least one location of the brain of the person.

In some embodiments, a method of characterizing brain health metrics of a person is provided. The method includes receiving information characterizing, at least in part, a brain structural composition and a functional architecture of a brain of a person, wherein the information includes first data including passive data and active data recorded from the person, processing the first data to extract, at least in part, metrics of performance and efficiency of brain and cognitive systems of the person, and defining, based on the metrics of performance and efficiency of the brain and cognitive systems of the person, an intervention to modulate brain activity of the brain of the person.

In one aspect, the information is collected via a wearable device, a personal computer or a portable headset. In another aspect, the information is collected via at least one electrophysiology technique. In another aspect, the at least one electrophysiology technique includes electroencephalography. In another aspect, the information is collected via at least one neuroimaging method. In another aspect, the at least one neuroimaging method includes one or more of magnetic resonance imaging (MRI), positron emission tomography (PET) or near infrared spectroscopy. In another aspect, the information is from multiple sources, and processing the first data to extract, at least in part, metrics of performance and efficiency of brain and cognitive systems of the person includes harmonizing, using an algorithm, the information from multiple sources into a digital twin. In another aspect, defining an intervention to modulate brain activity of the brain of the person comprises using the digital twin to define an intervention that enhances cognition and brain health of the brain. In another aspect, defining an intervention to modulate brain activity of the brain of the person includes using the digital twin to define an intervention that treats one or more neurological or psychiatric condition.

In another aspect, the method further includes using the digital twin to predict a trajectory of a neurological or psychiatric disease, wherein defining an intervention to modulate brain activity of the brain of the person comprises defining the intervention based, at least in part, on the predicted trajectory. In another aspect, the neurological disease is Alzheimer's disease, dementia, or brain cancer. In another aspect, the method further includes receiving second data sensed by a wearable device or a brain activity recording device, and updating the digital twin based, at least in part, on the second data. In another aspect, defining an intervention to modulate brain activity of the brain of the person includes using the digital twin to set one or more parameters for noninvasive brain stimulation. In another aspect, defining an intervention to modulate brain activity of the brain of the person includes using the digital twin to define personalized learning trajectories for skill acquisition. In another aspect, defining an intervention to modulate brain activity of the brain of the person comprises using the digital twin to derive measures of brain plasticity and resilience.

In another aspect, the received information is formalized as a network, and processing the first data to extract, at least in part, metrics of performance and efficiency of brain and cognitive systems of the person includes extracting characteristics of the network using graph theory metrics and network control theory metrics. In another aspect, the received information is formalized as a network, and processing the first data to extract, at least in part, metrics of performance and efficiency of brain and cognitive systems of the person comprises extracting characteristics of the network by analyzing at least one of an amplitude, shape or frequency of the first data. In another aspect, processing the first data to extract, at least in part, metrics of performance and efficiency of brain and cognitive systems of the person includes calculating the structural, functional, and dynamic resilience of the brain. In another aspect, processing the first data to extract, at least in part, metrics of performance and efficiency of brain and cognitive systems of the person includes computing a structural connectome of the brain. In another aspect, the method further includes monitoring disease progression and predicting levels of neuroinflammation in the brain based, at least in part, on the structural connectome of the brain. In another aspect, the method further includes monitoring demyelination in the brain and/or identifying white matter lesions in the brain based, at least in part, on the structural connectome of the brain.

In another aspect, the information includes information on an emotional state of the person recorded by a camera, the method further comprising storing a non-fungible token representing the information including the information on the emotional state of the person. In another aspect, the method further includes creating an emotional virtual agent based on the information on the emotional state of the person. In another aspect, the method further includes using the emotional virtual agent to monitor and support a patient with dementia or to enhance the brain health of a healthy person. In another aspect, the method further includes guiding at least one cognitive or behavioral intervention based on the information on the emotional state of the person to improve brain health. In another aspect, the method further includes guiding a dose of a therapeutic intervention based on the information on the emotional state of the person.

In another aspect, the information includes spontaneous and/or stimulus-driven eye movements recorded via a camera, the method further including storing a non-fungible token representing an analysis of the spontaneous and/or stimulus-driven eye movements. In another aspect, the spontaneous and/or stimulus-driven eye movements are collected during execution of a cognitive task by the person. In another aspect, the method further includes assessing brain health and overall cognitive performance of the person based on the non-fungible token. In another aspect, the method further includes assessing improvement in cognitive performance and learning capabilities of the person based on the non-fungible token. In another aspect, the spontaneous and/or stimulus-driven eye movements are collected during playing a videogame by the person, the method further including using the non-fungible token to assess in-game performance of the person.

In some embodiments, a method of stimulating one or more brain regions of a person to guide a state of the brain of the person to a target state is provided. The method includes receiving non-invasive brain stimulation parameters, wherein the non-invasive brain stimulation parameters are determined using information included in a dynamic multilayer digital twin (DMDT) associated with the brain of the person, controlling at least one non-invasive brain stimulation device according to the non-invasive brain stimulation parameters to stimulate the one or more brain regions of the person, and receiving feedback regarding an effect of the stimulation of the one or more brain regions to determine whether the state of the brain of the person is in the target state.

In one aspect, the at least one non-invasive brain stimulation device is configured to provide one or more of transcranial electrical stimulation, transcranial magnetic stimulation or transcranial focused ultrasound stimulation. In another aspect, the at least one non-invasive brain stimulation device is configured to provide sensory stimulation, the sensory stimulation including audio stimulation and/or visual stimulation. In another aspect, the sensory stimulation is embedded within a multimedia content, the multimedia content comprising a movie, a song, a television show, a videogame or a virtual reality application. In another aspect, the at least one non-invasive brain stimulation device is configured to provide sinusoidal stimulation of patterns of noise bursts applied at a specific frequency or random noise stimulation limited to a specific frequency band. In another aspect, the method further includes providing the person with a non-invasive stimulation intervention sequentially or simultaneously with controlling the at least one non-invasive stimulation device to stimulate the one or more brain regions of the person. In another aspect, the non-invasive stimulation intervention comprises cognitive rehabilitation or cognitive training interventions including videogames. In another aspect, the non-invasive stimulation intervention comprises a behavioral intervention or a physical training intervention. In another aspect, the non-invasive stimulation intervention comprises a drug intervention.

In another aspect, controlling the at least one non-invasive brain stimulation device to stimulate the one or more brain regions of the person includes stimulating the one or more brain regions to selectively modify specific brain connections between the one or more brain regions. In another aspect, controlling the at least one non-invasive brain stimulation device to stimulate the one or more brain regions of the person includes stimulating the one or more brain regions to increase brain plasticity and/or accelerate a rate of learning. In another aspect, the one or more brain regions are defined based on data associated with the person. In another aspect, the one or more brain regions are defined based on group-level data. In another aspect, the one or more brain regions are part of a functional brain network. In another aspect, the one or more brain regions include subcortical brain structures. In another aspect, the one or more brain regions are defined based on neuroimaging data. In another aspect, the neuroimaging data comprises functional MRI data. In another aspect, the neuroimaging data comprises diffusion MRI data. In another aspect, the neuroimaging data comprises structural MRI data. In another aspect, the neuroimaging data comprises Positron Emission Tomography (PET) data. In another aspect, the neuroimaging data comprises Near Infrared Spectroscopy data. In another aspect, the one or more brain regions are defined based on electrophysiology data. In another aspect, the electrophysiology data includes scalp electroencephalography data. In another aspect, the one or more brain regions are defined via mapping of white matter tracts in the brain of the person. In another aspect, the one or more brain regions are regions connected or disconnected to a brain cancer. In another aspect, the one or more brain regions are regions responsible for sleep induction.

In some embodiments, a method for determining health and performance of a brain of a person is provided. The method includes sensing a plurality of evoked potentials in response to first non-invasive stimulation of each location of a plurality of locations in the brain of the person, determining, based at least in part on at least one characteristic of the plurality of evoked potentials, personalized stimulation parameters for the person, wherein the personalized stimulation parameters include a location of stimulation and one or more stimulation characteristics, providing second non-invasive stimulation to the person based on the personalized stimulation parameters, and receiving feedback regarding the effect of the second non-invasive stimulation to extract metrics of performance and brain health.

In one aspect, the method further includes controlling a non-invasive stimulation device to provide the first non-invasive stimulation to each location of the plurality of locations in the brain of the person. In another aspect, providing the first non-invasive stimulation includes providing at least one of transcranial electrical stimulation, transcranial magnetic stimulation or transcranial focused ultrasound stimulation. In another aspect, providing the first non-invasive stimulation comprises providing at least one sensory or cognitive stimulus. In another aspect, the personalized stimulation parameters are determined based, at least in part, on a comparison of the at least one characteristic of the plurality of evoked potentials with spontaneous activity in the brain of the person. In another aspect, the first non-invasive stimulation is delivered to multiple of the plurality of locations simultaneously or in a predefined sequence. In another aspect, the plurality of locations are arranged in a grid for systematic spatial assessment of brain dynamics. In another aspect, the at least one characteristic of the plurality of evoked potentials comprises a peak magnitude. In another aspect, determining the personalized stimulation parameters includes selecting the location of stimulation as the location of the plurality of locations associated with a largest peak magnitude of the plurality of evoked potentials. In another aspect, the plurality of locations include locations in a brain network, the brain network comprising one or more of the Default Mode Network, the Fronto-Parietal control Network, the Sensorimotor Network, the Anterior Salience Network, the Dorsal Attention Network, the Ventral Attention Network, the Visual Network, the Auditory Network, or the Language Network.

In another aspect, the method further includes treating or ameliorating a neurological or psychiatric disease by providing the second non-invasive stimulation to the brain of the person based on the personalized stimulation parameters. In another aspect, the neurological or psychiatric disease is Alzheimer's Disease. In another aspect, the neurological or psychiatric disease is Mild Cognitive Impairment (MCI). In another aspect, the neurological or psychiatric disease is Frontotemporal Dementia. In another aspect, the neurological or psychiatric disease is one of Depression (DEP), Schizophrenia (SCZ), Autism (AUT), Attention Deficit & Hyperactivity Disorder (ADHD), bipolar disorder (BP), amyotrophic lateral sclerosis (ALS), Parkinson's disease (PD), traumatic brain injury (TBI), Insomnia (INS), Disorder of Consciousness (DOC), headache (HD), multiple sclerosis (MS), Stroke (STR), or brain tumors (BT). In another aspect, the neurological or psychiatric disease is characterized by memory deficits. In another aspect, the neurological or psychiatric disease is characterized by deficits in cognitive control. In another aspect, the neurological or psychiatric disease is characterized by decrease of functional independence. In another aspect, the neurological or psychiatric disease is brain cancer. In another aspect, the neurological or psychiatric disease is a brain tumor. In another aspect, the second non-invasive stimulation to the brain of the person is provided before surgery to inform surgical planning associated with removal of at least a portion of the brain tumor. In another aspect, the second non-invasive stimulation to the brain of the person is provided after surgery to detect recurrence of the brain tumor.

In another aspect, the method further includes providing a drug intervention affecting the central nervous system to the person, wherein the drug intervention is provided sequentially or simultaneously with the second non-invasive stimulation. In another aspect, the method further includes providing a cognitive assessment, cognitive training or cognitive enhancement intervention to the person, wherein the cognitive assessment, cognitive training or cognitive enhancement intervention is provided sequentially or simultaneously with the second non-invasive stimulation. In another aspect, the method further includes providing a behavioral intervention to the person, wherein the behavioral intervention is provided sequentially or simultaneously with the second non-invasive stimulation. In another aspect, the method further includes providing third non-invasive stimulation to the person, wherein the third non-invasive stimulation is provided sequentially or simultaneously with the second non-invasive stimulation. In another aspect, the third non-invasive stimulation is transcranial electrical stimulation.

In some embodiments, a method of creating a plasticity-inducing intervention for a person is provided. The method includes receiving information characterizing a brain state and a plasticity level of the person, wherein the information includes electrophysiological, behavioral and neuroimaging data recorded from the person, identifying a plasticity-inducing intervention for the person, the plasticity-inducing intervention including one or more brain stimulation targets, controlling at least one non-invasive brain stimulation device to deliver stimulation to the one or more brain stimulation targets, and receiving feedback regarding an effect of the stimulation of the one or more brain stimulation targets to determine whether the plasticity level of the brain of the person has changed.

In one aspect, the plasticity level of the person is measured via brain and cognitive metrics obtained using a dynamic multilayer digital twin (DMDT) of the brain of the person. In another aspect, identifying a plasticity-inducing intervention is based on an enriched environment determined based on the DMDT of the brain of the person. In another aspect, the enriched environment is a virtual, augmented or mixed reality environment. In another aspect, the enriched environment is delivered in the form of a room equipped with sensory stimulation devices and data recording devices. In another aspect, the enriched environment is delivered in the form of a videogame. In another aspect, the enriched environment is represented by a dual task platform.

In another aspect, the plasticity-inducing intervention is configured to target the perineuronal net and extracellular matrix of the brain of the person. In another aspect, the plasticity-inducing intervention is based on modulation of CSPGs via injection of ChABC. In another aspect, the plasticity-inducing intervention is based on modulation of CSPGs via manipulation of BDNF. In another aspect, the plasticity-inducing intervention is based on modulation of CSPGs via ketamine administration. In another aspect, the plasticity-inducing intervention is based on drugs acting on the GABAergic circuitry of the brain. In another aspect, the plasticity-inducing intervention comprises using a drug to modulate one or more of PNN, ECM or CSPGs. In another aspect, the plasticity-inducing intervention is based on noninvasive brain stimulation methods. In another aspect, the noninvasive brain stimulation methods include protocols to induce fast oscillatory activity in the brain in the gamma frequency band. In another aspect, the noninvasive brain stimulation methods include protocols to induce activity in the gamma frequency band to activate inhibitory interneurons and glia cells. In another aspect, the noninvasive brain stimulation methods include protocols to induce gamma activity in the brain, the noninvasive brain stimulation methods including transcranial alternating current stimulation (tACS), transcranial pulsed gamma stimulation (tPGS), and/or narrow band transcranial random noise stimulation (nb-tRNS).

In another aspect, the plasticity-inducing intervention is applied in patients with neurological and psychiatric conditions. In another aspect, the plasticity-inducing intervention is applied in an athlete in order to boost performance and response to training. In another aspect, the plasticity-inducing intervention is applied to boost sleep quality and memory consolidation. In another aspect, the plasticity-inducing intervention is applied in patients with post-traumatic stress disorder (PTSD) to facilitate the manipulation and removal of traumatic memories and promote a healthy brain state. In another aspect, the plasticity-inducing intervention is applied in astronauts to increase learning and retention of skills, and to facilitate protection from space-flight associated brain and cognitive changes. In another aspect, the plasticity-inducing intervention is applied during sleep to enhance memory consolidation and overall brain plasticity.

In some embodiments, a method of enhancing cognitive performance and learning abilities by stimulation of the brain of a person in a predefined sequence is provided. The method includes receiving information characterizing an initial brain state of the person, wherein the information includes electrophysiological and cognitive data recorded from the person, defining an optimal sequence of cognitive changes to be induced to change a state of the person from the initial brain state to a target brain state, delivering, based on the optimal sequence of cognitive changes to be induced, one or more cognitive modulators targeting specific brain structures and cognitive functions, and receiving feedback regarding the effect of the delivered one or more cognitive modulators to determine whether a state of the brain of the person is in the target brain state.

In one aspect, the optimal sequence of cognitive modulators is defined in order to reach an Optimal Learning State (OLS). In another aspect, the OLS is defined on the basis of a dynamic multilayer digital twin (DMDT) of the brain of the person. In another aspect, the target brain state is a state of high cognitive performance defined by a specific pattern of activation and deactivation of brain regions based on a neuroimaging or electrophysiology map. In another aspect, the optimal sequence of cognitive modulators is combined with a brain modulator to increase brain excitability, modulate plasticity and/or increase brain efficiency or modularity. In another aspect, the brain modulator is a form of non-invasive stimulation. In another aspect, the one or more cognitive modulators include cognitive training acting on sensory and cognitive function. In another aspect, the target brain state is a brain state in which a rate of learning is accelerated. In another aspect, the rate of learning is associated with learning a sensorimotor skill including one or more of playing a musical instrument, augmenting memory performance, or learn a coding language. In another aspect, the rate of learning is associated with learning a rehabilitation skill in a patient with a neurological or psychiatric condition. In another aspect, the rate of learning is associated with improving physical and/or mental performance associated with athletics. In another aspect, the rate of learning is associated with improving educational performance. In another aspect, the rate of learning is associated with a cognitive-behavioral therapy, a verbal therapy, or a dynamic therapy. In another aspect, the rate of learning is associated with enhancing convergent thinking in the form of fluid intelligence and abstract reasoning. In another aspect, the rate of learning is associated with enhancing divergent thinking in the form of creativity and insight abilities. In another aspect, the rate of learning is associated with enhancing general intelligence by targeting brain regions and networks of a multilayer Convergent-Divergent thinking (CDt) model of human cognition.

In some embodiments, a system for administering a brain optimization procedure is provided. The system includes a data collection platform configured to collect and store data of an individual used for identifying an optimal optimization target, a data analysis platform configured to process the data of the individual and derive optimal stimulation parameters, and a database including the data of the individual collected before and/or during a brain stimulation treatment based on the optimal stimulation parameters.

In one aspect, the system is accessible to a clinician prescribing the brain stimulation treatment. In another aspect, the system is accessible to an end user via a portable computing device. In another aspect, the data analysis platform is a cloud-based computing platform in communication with the data collection platform via at least one network. In another aspect, the system is controlled via a blockchain environment.

In some embodiments, a system for recording and modulating brain activity in a person is provided. The system includes a stimulation device configured to provide non-invasive stimulation to each location of a plurality of locations in a brain region of the person, a sensor device configured to sense, in response to the non-invasive stimulation provided by the stimulation device, at least one evoked response, and a computer processor configured to select as a personalized stimulation target, one of the locations of the plurality of locations that is suitable for modulating brain activity via non-invasive stimulation, wherein the selection is based on at least one characteristic of the at least one evoked response.

In one aspect, the stimulation device and the sensor device are included as part of a portable device in the form of a wearable headset. In another aspect, the portable device is configured to be controlled by a separate computing device allowing for remote control of the portable device. In another aspect, the stimulation device is configured to provide at least one of transcranial electrical stimulation, transcranial magnetic stimulation, or transcranial focused ultrasound stimulation to the personalized stimulation target. In another aspect, the stimulation device is configured to treat or ameliorate a neurological or psychiatric disease of the person by providing the non-invasive stimulation to the selected personalized stimulation target. In another aspect, the neurological or psychiatric disease is Alzheimer's disease. In another aspect, the neurological or psychiatric disease is Frontotemporal Dementia. In another aspect, the neurological or psychiatric disease is Mild Cognitive Impairment (MCI). In another aspect, the neurological or psychiatric disease is brain cancer. In another aspect, the neurological or psychiatric disease is glioma. In another aspect, the neurological or psychiatric disease is depression. In another aspect, the neurological or psychiatric disease is ADHD. In another aspect, the neurological or psychiatric disease is a sleep disorder. In another aspect, the neurological or psychiatric disease is characterized by alterations of brain networks, such as Depression (DEP), Schizophrenia (SCZ), Autism (AUT), Attention Deficit & Hyperactivity Disorder (ADHD), bipolar disorder (BP), amyotrophic lateral sclerosis (ALS), Parkinson's disease (PD), traumatic brain injury (TBI), Insomnia (INS), Disorder of Consciousness (DOC), headache (HD), multiple sclerosis (MS), Stroke (STR), and brain tumors (BT). In another aspect, the neurological or psychiatric disease is characterized by memory deficits. In another aspect, the neurological or psychiatric disease is characterized by deficits in cognitive control. In another aspect, the neurological or psychiatric disease is characterized by decrease of functional independence.

In another aspect, the non-invasive stimulation provided to the selected personalized stimulation target is combined with a drug intervention affecting the central nervous system, delivered sequentially or simultaneously. In another aspect, the non-invasive stimulation provided to the selected personalized stimulation target is combined with one or more of a cognitive assessment, cognitive training, or cognitive enhancement intervention, delivered sequentially or simultaneously. In another aspect, the non-invasive stimulation provided to the selected personalized stimulation target is combined with a behavioral intervention, delivered sequentially or simultaneously. In another aspect, the non-invasive stimulation provided to the selected personalized stimulation target is combined with a different non-invasive stimulation intervention, delivered sequentially or simultaneously, including transcranial electrical stimulation.

In another aspect, the computer processor is further configured to elaborate a sequence of combined automated and semiautomated signal processing algorithms installed on a local hardware, resulting in a specific indication for one or more of stimulation location, frequency, or intensity. In another aspect, the computer processor is further configured to elaborate a sequence of combined automated and semiautomated signal processing algorithms installed on remote hardware with connectivity capabilities, resulting in a specific indication for stimulation location, frequency, or intensity. In another aspect, the sensor device includes electrodes configured to be placed on a scalp of the person. In another aspect, the sensor device includes electrodes configured to be placed inside an ear of the person. In another aspect, the sensor device includes electrodes configured to be placed on a face of the person. In another aspect, the sensor device includes high-density electrodes based on carbon nanotubes placed inside a stimulation cap placed on the head of the person. In another aspect, the system is configured to be used within the metaverse.

In some embodiments, a method of collecting neurophysiological and cognitive-behavioral data of an individual in order to characterize brain health metrics is provided. The method includes receiving information characterizing, at least in part, the brain structural composition and functional architecture of an individual, wherein the information includes passive and active data recorded from the person, processing the data to extract, at least in part, metrics of performance and efficiency of the brain and cognitive systems, and defining an intervention to modulate brain activity and optimize performance and efficiency of the brain.

In one aspect, the information is received from multiple sources and is harmonized via an algorithm and combined into metrics of brain performance and brain health as part of a DMDT. In another aspect, the information included in the DMDT is used to optimize interventions aimed at enhancing cognition and brain health via the DARWIN algorithm described herein. In another aspect, the information included in the DMDT is used to optimize interventions aimed at treating neurological or psychiatric conditions via the DARWIN algorithm. In another aspect, the information included in the DMDT is used to predict the trajectory of a neurological or psychiatric disease, in the form of diagnostic and prognostic markers. In another aspect, the information included in the DMDT is updated on the basis of new data collected via wearables or a brain activity recording device. In another aspect, the information included in the DMDT is used to set parameters for noninvasive brain stimulation. In another aspect, the information included in the DMDT is used to create avatars and content for gaming and metaverse applications. In another aspect, the information included in the DMDT is used to define personalized learning trajectories for skill acquisition. In another aspect, the information included in the DMDT is used to derive measures of brain plasticity and resilience.

In another aspect, the information included in the DMDT is stored as a Non-fungible token (NFT) and subsequently used to track the progress of a therapeutic, restorative or enhancement intervention. In another aspect, the information included in the DMDT is stored as a Non-fungible token (NFT) and subsequently used to define brain and cognitive stimulation approaches. In another aspect, the information included in the DMDT is stored as a Non-fungible token (NFT) and subsequently used to compare individual DMDT to a population-level DMDT and calculate a distance matrix. In another aspect, the information included in the DMDT is stored as a Non-fungible token (NFT) and used as a biometric data for encryption and cybersecurity applications. In another aspect, the information included in the DMDT is stored as a Non-fungible token (NFT) and used as an asset for digital commerce and financial transactions in the metaverse. In another aspect, the information included in the DMDT is stored as a Non-fungible token (NFT) and used as an asset to generate personalized digital content in gaming applications, including but not limited to gears, weapons, and outfits. In another aspect, a specific pattern of brain activity is stored as a Non-fungible token (NFT) and used as a password for encryption and cybersecurity applications in the context of online business and financial transactions.

In another aspect, a specific pattern of brain activity related to a specific stimulus is stored as a Non-fungible token (NFT). In another aspect, an individual cognitive architecture, including but not limited to information on brain networks dynamics and cognitive processing strategies, is stored as an NFT. In another aspect, an individual cognitive architecture is used as a target for cognitive enhancement, cognitive training and psychotherapy applications. In another aspect, an individual mind wandering pattern, including but not limited to a sequence of brain states recorded in relation to a specific event or stimuli, is stored as an NFT. In another aspect, an individual mind wandering pattern is used as a target for cognitive enhancement, cognitive training and psychotherapy applications.

### BRIEF DESCRIPTION OF DRAWINGS

The advantages of the invention, together with further advantages, may be better understood by referring to the following description taken in conjunction with the accompanying drawings. The drawings are not necessarily to scale, and emphasis is instead generally placed upon illustrating the principles of the invention.

FIG. 1 schematically illustrates exemplary components of an neuromodulating and enhancement platform, in accordance with some embodiments of the present disclosure.

FIG. 2 shows an example cloud computing and blockchain architecture for use with some embodiments of the present disclosure.

FIGS. 3A-3C schematically show multi-layer NFT creation from neural data, in accordance with some embodiments of the present disclosure.

FIG. 4 schematically shows component of a system for capturing information about an individual, in accordance with some embodiments of the present disclosure.

FIG. 5 illustrates an example data analysis pipeline including data processing components that may be used in accordance with some embodiments of the present disclosure.

FIG. 6A shows an example of individual differences in state or trait transitioning, in accordance with some embodiments of the present disclosure.

FIG. 6B shows metrics of state transitioning for a group of subjects, in accordance with some embodiments of the present disclosure.

FIG. 7 shows results of a study in which perturbation of the brain was performed to improve resilience measures of the brain, in accordance with some embodiments of the present disclosure.

FIG. 8 shows graph measures representing integration and segregation of information processing, in accordance with some embodiments of the present disclosure.

FIGS. 9A-9C schematically show a process for mapping a connectivity profile of a brain tumor, in accordance with some embodiments of the present disclosure.

FIG. 10 schematically shows components of a system for monitoring a brain tumor, in accordance with some embodiments of the present disclosure.

FIGS. 11A and 11B schematically show an example of dynamic decomposition for psycho-cognitive assessment, in accordance with some embodiments of the present disclosure.

FIG. 12 shows brain regions identified as supporting abstract reasoning and problem solving in humans that are used to inform neuromorphic artificial intelligence agents, in accordance with some embodiments of the present disclosure.

FIG. 13 shows brain networks identified as supporting abstract reasoning and problem solving in humans that are used to inform neuromorphic artificial intelligence agents, in accordance with some embodiments of the present disclosure.

FIGS. 14A-14C show main clusters of brain networks supporting intelligence in humans that are used to inform neuromorphic artificial intelligence agents, in accordance with some embodiments of the present disclosure.

FIGS. 15A-15B show activation foci and brain connectivity patterns, in accordance with some embodiments of the present disclosure.

FIGS. 16A-16D show functional connectivity of overlapping nodes across convergent and divergent thinking in a brain that are used to inform neuromorphic artificial intelligence agents, in accordance with some embodiments of the present disclosure.

FIGS. 17A-17B show example analysis, connectivity and cognition output from the DARWIN module of the architecture of FIG. 1, in accordance with some embodiments of the present disclosure.

FIGS. 18 and 19 schematically shows components of a brain-to-command engine module used to convert information from brain data into coding language for brain-computer interface applications, in accordance with some embodiments of the present disclosure.

FIG. 20 shows components of a neuromorphic oscillatory multiscale adaptive artificial intelligence module inspired by brain activity, in accordance with some embodiments of the present disclosure.

FIGS. 21A-21F show examples of controlled perturbations and corresponding brain responses used to map brain activity, in accordance with some embodiments of the present disclosure.

FIGS. 22A-22C show results of a study in which transcranial magnetic stimulation (TMS) was delivered over multiple networks of the brain based on individual MRI and fMRI data collected in patients with Alzheimer's disease, in accordance with some embodiments of the present disclosure.

FIGS. 23A-23B show evoked oscillatory activity in Alzheimer's patients after TMS, in accordance with some embodiments of the present disclosure.

FIGS. 24A-24B show network-level response to perturbation used to perform fingerprinting of brain activity, in accordance with some embodiments of the present disclosure.

FIGS. 25A-25E show clinical applications of combined neuroplasticity protocols and noninvasive brain stimulation to induce brain plasticity, in accordance with some embodiments of the present disclosure.

FIGS. 26A-26B show enhancement of brain plasticity via brain stimulation, in accordance with some embodiments of the present disclosure.

FIGS. 27A-27B show clusters of brain activation and deactivation in anticipation and/or during placebo response, in accordance with some embodiments of the present disclosure.

FIGS. 28A-28B show brain networks corresponding to placebo activity, in accordance with some embodiments of the present disclosure.

FIG. 29 shows a Modular, Adaptive, Neurological approach to Psychological Change (MANP) hierarchical approach to increase the efficacy of mental health interventions, in accordance with some embodiments of the present disclosure.

FIG. 30 shows a schematic representation of an example software architecture for a CLARITY module for a virtual assistant and caregiver, in accordance with some embodiments of the present disclosure.

FIG. 31 is a plot that illustrates modulation of brain and cognitive plasticity as a function of manipulation of environmental complexity in a virtual reality environment, in accordance with some embodiments of the present disclosure.

FIG. 32A shows a network of brain regions identified as a potential target for neuromodulation of motion sickness, vertigo and nausea during VR applications, in accordance with some embodiments of the present disclosure.

FIG. 32B shows an optimized electrical stimulation pattern for a tACS application to reduce nausea and vertigo during VR applications, in accordance with some embodiments of the present disclosure.

FIG. 32C schematically shows tACS electrodes placed underneath an elastic band holding the headset and in proximity of the right and left ear of person, in accordance with some embodiments of the present disclosure.

FIGS. 33A and 33B show results of a study in which the brain of a person was stimulated in a particular manner to reduce the sensation of motion sickness, in accordance with some embodiments of the present disclosure.

FIGS. 34A-C show responses to Adaptive Brain State Optimization (ABSO), in accordance with some embodiments of the present disclosure.

### DETAILED DESCRIPTION

The inventor has recognized and appreciated that quantification of the complexity of the brain is important for understanding and modifying human cognitive abilities and behavioral patterns, enhancing brain function, identifying personalized interventions to promote mental health and healthy aging, and providing individuals with knowledge about their brain capacity for learning, adapting, and evolving. Some embodiments of the present disclosure relate to techniques for mapping, characterizing, predicting and/or optimizing brain function, using an integrated software-hardware platform. Systems and methods include data analysis and visualization tools, algorithms for estimation of brain potential and corresponding strategies for brain, cognitive and behavioral enhancement, hardware for data collection and neuromodulation, and application-specific algorithms for the generation of digital assets based on individual brain activity features. The data processing techniques and algorithms described herein have a wide range of applications including, but not limited to, the creation of digital twins of a patient or a healthy individual, digital health biometrics, solutions to augment brain plasticity and learning abilities, algorithms for brain state-state and trait-trait transition via personalized neuromodulation, enhancement of brain health and cognitive performance, deriving personalized interventions to manipulate brain activity and alleviate symptoms in patients with neurological and psychiatric diseases, acceleration of skill acquisition, and for use in creating brain-inspired game mechanics and content for the gaming industry and the metaverse.

The inventor has recognized that even when solutions to map brain complexity and generate models of brain activity and behavior will become available, solutions to accurately manipulate brain activity via controlled perturbations will be needed. Data collected on individual brains may be used to generate computational evolutionary biology models and identify the best strategies to improve brain performance and behavior, coupled with interventions to modify the brain's ability to learn and evolve, e.g., via brain plasticity. The inventor has recognized the need for a new platform for data collection, harmonization, analysis and simulation of brain, cognitive and behavior data. Some systems and methods of the present disclosure may be referred to herein as a Neuromodeling and Enhancement Platform (NEP), encompassing software and hardware solutions to perform one or more of brain fingerprinting, estimation of brain potential, development of digital health assessment tools based on individual brain and cognitive features, and brain optimization.

Some embodiments relate to a platform (e.g., NEP) for data collection, data harmonization, data processing and manipulation, for the creation of digital replicas of the human brain and in-silico models that can be used to simulate brain and human cognitive behavior. NEP may include software and hardware approaches to, at least in part: characterize individual brain complexity and generate digital twins of healthy and pathological brain function; generate an artificial intelligence agent reflecting an individual's brain dynamics and cognitive architecture (neuromorphic artificial intelligence); personalize solutions for state-state and trait-trait brain transition used to estimate brain potential and create individualized trajectories of brain/cognitive optimization; derive cognitive/brain enhancement protocols leveraging cortical plasticity mechanics; promote interventions aimed at maintaining brain health as well as therapeutics protocols for patients with neurological and psychiatric conditions; generate assets for the metaverse and gaming industry by using neuro-data within open-and closed-loop environments. These solutions may have direct applications in various fields, including the medical and human performance field, as well as the metaverse where a new generation of applications and assets can be created based on brain activity and an individual's brain digital twin(s).

An overview of example components of the NEP framework and their specific applications are described in more detail below. The framework includes original data and algorithms involving principles and tools to modulate brain plasticity, techniques to map the human connectome, science of learning applied to videogames and cognitive training, virtual reality applications, and novel tools for noninvasive brain stimulation of the healthy and diseased brain. Following the general discussion of NEP, details on procedures and related data are described in greater detail for each example component.

### Mapping, Predicting and Modulating Brain and Behavior

The platform described herein provides for mapping the complexity of an individual brain, opening scenarios where such “brain-prints” may be used to define or guide personalized solutions across multiple domains and markets, ranging from discovery of novel biomarkers of disease to the definition of personalized therapies within the precision medicine framework, to the creation of brain-inspired artificial intelligence, implementation of personalized training and dietary regimes, to personalized cognitive and behavioral to accelerate skill acquisition, to algorithms for creating brain-inspired game mechanics and content for the videogaming industry and the metaverse.

Once such complexity is mapped and documented in digital form, models on how the brain of a given individual or a group of individuals respond to external or internal perturbation can be derived. In this context, solutions to quantify a brain's ability to adapt to change (often referred to as “neural plasticity” or simply “plasticity”), form memories, evolve in response to experience, as well as the brain's cognitive potential, become possible and can be used to improve the aforementioned applications of brain-prints. Recently, knowledge has accumulated on methods and approaches to map individual brains via neuroimaging and electrophysiology techniques and modify brain structure and function especially with the help of noninvasive brain stimulation techniques. Although basic principles on how to map brain structure and function effectively have been proposed, as well as behavioral or brain stimulation solution to boost brain's ability, methods and principles to parametrize such processes and configure the necessary steps and changes to transition the brain from a given state A to another state B (e.g., from pathology to normal functioning, from a relaxed state to an abstract reasoning state) are not available. The system and methods described herein allow, for instance, to define:
(i) state-to-state transition steps for each brain (e.g., for a given brain with baseline state A, the best strategy to reach state B may be to increase overall brain plasticity levels and increase network modularity; whereas for a brain with baseline state C, the focus may be to increase flexibility and cortical excitability levels);
(ii) an order of the state-to-state transition steps (e.g., first change stronger connections, weak connections will follow; focus should be first on increasing left hemisphere connections, then right ones);
(iii) the intensity/magnitude of stimulus needed to effect each change based on an individually defined brain state;
(iv) optimal temporal framework according to a brain's ability to change and adapt (e.g., deliver a given type of brain stimulation every day, week, multiple times a day, overnight, interleaved with cognitive and/or behavioral training).

By parametrizing such steps and notions, and estimating how much “change” different interventions can have on the brain (e.g., cognitive training can change brain connectivity by 0.5% every session; meditation increases spectral power of a given oscillation by 1% every 10 hours of stimulation), it becomes possible to predict the number of “steps” required to migrate from state A to state B. A specific module of NEP (also referred to herein as the “DARWIN” module), includes solutions for such analysis and model building, with application in both clinical and non-clinical contexts.

### Increasing a Brain's Capacity to Learn and Change

Opportunities to modulate brain states and traits also leverage knowledge on brain plasticity and related opportunities to reactivate/manipulate plasticity levels in the human brain via pharmacological, behavioral and cognitive approaches. In humans, brain plasticity peaks during early childhood, after which plasticity levels drop in parallel with the possibility of observing major modifications of brain wiring even in response to external stimuli. At the core of such change in a brain's ability to “learn” is, at least in part, a structural change in the perineuronal net (PNN), an extracellular matrix (ECM) structure rich in chondroitin sulfate proteoglycans (CSPGs). The major event that reduces adult CNS plasticity is the accumulation of extracellular matrix molecules, such as CSPGs, around somata and dendrites of neurons. The systems and methods described herein include algorithms and hardware to further amplify the effect of ECM/PNN modulation, thereby improving learning as well as brain physiology (e.g., brain resilience, flexibility, efficiency) and state-trait transition. Some of the systems and methods described herein combine interventions including PNN modulators with brain optimization and brain modifiers (including but not limited to noninvasive brain magnetic and electrical stimulation) as a platform to enhance brain physiology, increase cognitive functioning, accelerate recovery from injury and brain pathology, and promote brain health. The sections below on brain plasticity and learning applications of NEP provide further detail.

### Personalized Content Generation From Neuro-data

Information on brain activity, behavior and cognition for a given individual can be used in some embodiments for applications in the videogaming industry and the metaverse. For instance, individual brain activity can guide a virtual reality/augmented reality/extended reality (VR/AR/XR) experience, be used to generate assets and content based on raw and processed brain data acquired in real-time or processed offline, guide the automatic creation of “worlds” and corresponding physics based on brain activity and other physiological data (e.g., neuro-architecture). Similar techniques may apply to the gaming industry, where information on each individual player may be used to characterize in-game parameters and adaptive game dynamics, whereas information on each player's evolvability may be used to derive individual trajectories for in-game growth, avatar optimization, creation of personalized artificial intelligence (A. I.), and/or controlling other players or non-player characters (NPCs) in the game. The section below describing the NEUROCREATOR module provides additional details on how the NEP can be used for example metaverse and gaming applications.

Clinical Applications The systems and methods described herein include algorithms with applications in the medical field including, but not limited to, techniques for the identification of novel biomarkers or novel treatment targets based on brain evolvability and connectome analysis. Moreover, data acquired from each individual can be stored and later used as a template for trait-trait transition; for instance, a snapshot of brain activity obtained from a patient with dementia before the onset of clinical symptomatology (including but not limited to memory problems) can be used to guide a sensory, cognitive, or brain stimulation intervention aimed at restoring pre-pathology healthy brain dynamics. A similar application can be used to guide surgery and post-surgery recovery in patients with brain tumors. Similar applications can be used for neurological and/or psychiatric conditions including, but not limited to, post-traumatic stress disorder, and depression and anxiety disorders.

### Data Protection and Storage

Given the uniqueness of each individual human brain, the capacity of some of the techniques described herein to synthesize, quantify and model brain structure and function may allow for storing Non-fungible tokens (NFTs) based on individual brain/cognitive/behavioral data. NFTs may be used as a target brain state and trait for brain transitioning (e.g., go back to a high memory performance state), as a form of digital currency, as a template for the creation of digital avatars, as the wiring diagram of biologically-inspired artificial intelligence. Dynamic brain data collected via a portable device in specific, meaningful, life moments, could be stored and reproduced, with applications from art to gaming-related online trades. Systems and methods for collecting and storing data are also described herein. Data may be stored via a Data Management & Protection system (referred to herein as “DATANET”), responsible for data storage, quality assurance, and protected data sharing. DATANET may be based on a multikey, decentralized ledger technology (e.g., blockchain) algorithm with enhanced compression capabilities to allow storage and transfer of large DMDT and/or DARWIN files.

### Neuromorphic A.I.

Knowledge accumulated on brain functioning, its association with behavior and cognition, and strategies for brain enhancement and optimization, is also used in some embodiments to create neuromorphic artificial intelligence (A. I.). Group-level patterns of brain activity optimal for the solution of a given problem, or related to a particular skill set (e.g., high abstract reasoning performance) may be used to design A.I. tools for specific tasks, and generalized A.I. tools able to produce independent thinking and synthetic consciousness. In some embodiments, the A.I. tools may be based on knowledge related to computational and topological properties of brain function, including the role of oscillatory networks. In some embodiments, the A.I. tools may include assistive (e.g., conversational) agents to help patients with reduced functional independence [CLARITY], A.I. tools to integrate patients' psychotherapy treatments via personalized digital counseling, and A.I. tools for generative digital assets creation via natural language processing used for interactive videogame applications. The section below entitled NEURO-AI on neuromorphic A.I. applications of NEP provides additional details.

### EXAMPLE ARCHITECTURE

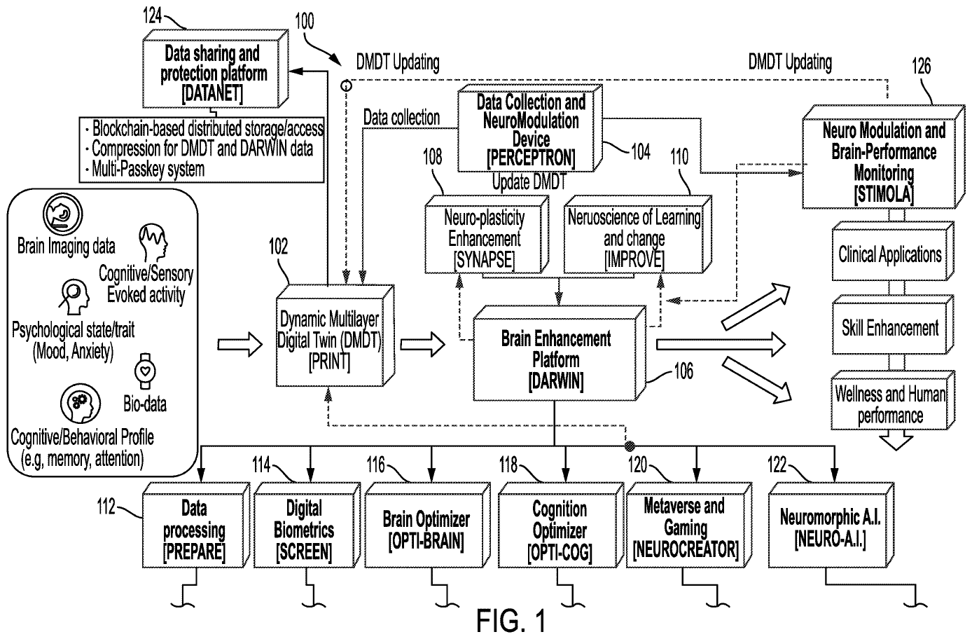

FIG. 1 schematically illustrates exemplary components of an NEP platform 100 in accordance with some embodiments of the present disclosure.
1) [BRAINPRINT] is a harmonization unit 102 used to characterize each individual user of the platform via a combination of multimodal data collected via dedicated or third-party systems;
2) [PERCEPTRON] is a monitoring and stimulation unit 104, in the form of a portable device, allowing for capturing brain data, physiological data, cognitive and behavioral performance data, and allowing for brain stimulation via multiple stimulation modalities including, but not limited to, sensory, electrical, or magnetic stimulation;
3) [DARWIN] is a brain enhancement platform 106 that includes a set of algorithms and computational tools for multipurpose analysis of BRAINPRINT data to estimate one or more of brain potential, brain evolutionary trajectory, brain plasticity levels and other properties related to brain health.

DARWIN modules include:
(i) [SYNAPSE] is a module 108 that includes systems and methods to measure, predict and modulate neuroplasticity, including behavioral and brain stimulation solutions to disrupt the perineuronal net (PNN) and reopen windows of plasticity in the human brain;
(ii) [IMPROVE] is a module 110 that includes systems and methods for enhancement/optimization of learning processes, and solutions for cognitive-behavioral enhancement, including knowledge on optimal brain targets for neuromodulation of specific cognitive functions and behaviors, and the optimal hierarchy of cognitive and behavioral steps necessary to accelerate learning;
(iii) [PREPARE] is a module 112 that includes algorithms for data processing and analysis, including but not limited to solutions to clean, preprocess, and analyze brain data from single individuals or a group of individuals, providing inputs for second-level algorithms such as OPTI-BRAIN, OPTI-COG and NEUROCREATOR, which are described in more detail below;
(iv) [SCREEN] is a digital biometrics platform 114, including but not limited to methods and tasks for assessment of cognitive, psychological and brain health, as well as the development of novel assessment tools based on DMDT data and principles from SYNAPSE, DARWIN and IMPROVE;
(v) [OPTI-BRAIN] is a module 116 that includes systems and methods for optimizing brain function, including but not limited to solutions to increase brain resilience, information processing, modularity, flexibility of brain networks; solutions also include applications to promote and guide transition from/to a given brain state or trait, and to identify optimal stimulation targets for therapeutic applications in neurological and psychiatric conditions;
(vi) [OPTI-COG] is a module 118 that includes systems and methods for assessing and enhancing human cognition, including approaches to modulate brain function and specific brain networks supporting specific cognitive abilities, such as abstract reasoning, memory and attention. The module also includes algorithms for the definition of cognitive assessment tools based on individual properties captured via DARWIN, including plasticity principles from SYNAPSE, learning principles from IMPROVE and brain properties quantified via OPTI-BRAIN. The modules compose a digital health platform for assessment and modulation of brain activity and cognition.
(vii) [NEUROCREATOR] is a module 120 that includes systems and methods for the generation of unique assets and content for the metaverse and videogaming industry based on individual features extracted via BRAINPRINT and processed via DARWIN;
(viii) [NEURO-AI] is a module 122 that includes systems and methods for the creation of neuromorphic artificial intelligence, including algorithms to generate A.I. tools resembling an individual's brain characteristics captured by DMDT or group-level characteristics of a particular cluster of individuals, and evolutionary algorithms to improve A.I. performance following data generated by DARWIN on human brain data.
4) [DATANET] is a data management & protection system 124 responsible for data storage, quality assurance, and protected data sharing. DATANET is based on a decentralized ledger technology (e.g., blockchain) algorithm with enhanced compression capabilities to allow storage and transfer of large DMDT and DARWIN files. DATANET includes a multi-passkey encryption for separate data access by commercial and non-profit institutions, allowing data owners to share their data to multiple users for different purposes, without ever sharing the totality of their data. DATANET includes a remuneration system for data owners to receive compensation based on their DMDT data usage, in perpetuity.
5) [STIMOLA] is a module 126 that includes a system and methods for manipulation of brain activity, cognition and behavior via neuromodulation; these include, but are not limited to, noninvasive brain stimulation techniques, cognitive training, videogames, and augmented, mixed and virtual reality tools;
6) [TRAINER] includes systems and methods for skill-specific enhancement, where information from BRAINPRINT and models from DARWIN modules are combined to program personalized training regimes maximizing learning and cognitive performance; these include, but are not limited to, language acquisition, musical training, visuomotor training, behavioral training, and cognitive therapy. TRAINER may be combined with methods from STIMOLA for additive effects.

Each of the enumerated components above are described in more detail below, and example datasets demonstrating the feasibility and functionality of said components and approaches are provided.

### WORKFLOW

A general example application where data flows through the NEP platform is described below:
1) Data collected from a person through PERCEPTRON and/or other wearable systems may be stored in a cloud-based database as part of BRAINPRINT. The data, including input from multiple modalities including, but not limited to, neuroimaging data (including, but not limited to, electroencephalography (EEG), magnetic resonance imaging (MRI), positron emission tomography (PET), and magnetoencephalography (MEG)), cognitive-behavioral measures (including, but not limited to, memory scores, language proficiency, reaction times, and emotional response to stimuli) and physiological data (including, but not limited to, heart-rate variability, galvanic skin response, and thermal face response), may be preprocessed for further analysis and extraction of features for the creation of a Dynamic Multilayer Digital Twin (DMDT). Preprocessing steps may include, for instance, removal of data recording artifacts, resampling, filtering, relabeling of signal traces and averaging; other preprocessing steps may include an algorithm for harmonization of data based on distribution fitting and alignment, re-weighting, normalization; feature selection based on adaptive machine learning (AML) may be used to identify features carrying unique information of a person through a comparison with population-average templates of data distribution/variability part of a Human Template Repository (HTR); preprocessed brain, physiological, cognitive and behavioral data may be used to extract performance indexes (e.g., Brain Plasticity Index, Learning Index) based on data collected at baseline; In some embodiments, the performance indices are updated with new incoming data collected via PERCEPTRON and/or other apparatus.
2) DMDT data are sent to the DARWIN module for, at least in part, the creation of predictive models, estimation of brain potential, extraction of features for content generation in the metaverse, and/or personalization of neuromodulatory interventions.
3) DMDT data are sent to PREPARE, for data preprocessing, harmonization and databasing; data are processed and formatted in order to allow for further analysis via the DARWIN module.
4) OPTI-BRAIN may perform simulations on DMDT data to establish the optimal pattern for enhancement of a specific cognitive function, for instance logical reasoning. In this example, OPTI-BRAIN uses the organization of cognitive and functional brain networks as a primary reference to estimate a person's potential for learning and identify optimal, personalized learning trajectories.
5) OPTI-BRAIN solutions may be further refined based on algorithms and proprietary knowledge on the neuroscience of learning (e.g., via IMPROVE) and neuroplasticity (via SYNAPSE); for instance,
   algorithmic data from IMPROVE describing the optimal sequence of enhancement steps to achieve general cognitive enhancement is applied to the original OPTI-BRAIN model; for instance, based on specific cognitive and brain qualities described in DMDT data and the initial analysis from OPTI-BRAIN, IMPROVE may suggest to first improve visuo-spatial attentional levels, followed by enhancement of two executive functions (e.g., inhibition and flexibility), followed by increase in semantic verbal fluency;
   the revised OPTI-BRAIN solution for enhancement of logical reasoning may be further constrained by SYNAPSE, on the basis of DMDT data describing individual qualities related to neuroplasticity; given low levels of brain plasticity indexed by EEG and cognitive data described in DMDT, SYNAPSE suggests a global neuromodulation intervention (e.g., a drug disrupting the peri-neuronal net) aimed at increasing global brain plasticity first, followed by more selective/focal modulation of plasticity (e.g., via transcranial electrical stimulation-tES) targeting the Fronto-Parietal Control Network (FPCN) and Dorsal Attention Network (DAN) of the brain before implementation of solutions suggested by IMPROVE;
6) Following input from IMPROVE and SYNAPSE, the OPTI-BRAIN model for enhancement of logical reasoning may be sent to STIMOLA for implementation, including, for example, execution of specific transcranial electrical stimulation (tES) protocols to boost plasticity, and cognitive training protocols for the enhancement of cognitive functions (e.g., executive functions, visuo-spatial attention);
7) The protocol for enhancement of logical reasoning may be implemented via STIMOLA, based on an algorithm assessing dose-response curves and identifying personalized training regimes. Brain, cognitive and behavioral data may be collected via PERCEPTRON;
8) STIMOLA may provide feedback to DARWIN on the efficacy of enhancement protocols, while also updating the individual's DMDT, which may be used to update OPTI-BRAIN solutions in a closed-loop fashion.
9) PERCEPTRON may be used to track brain, behavioral and cognitive changes during the enhancement period by constantly or periodically updating the individual's DMDT and informing DARWIN models.

A different (but possibly similar) workflow can be used in different scenarios. For instance, in some embodiments, DMDT data and DARWIN models are used to create personalized avatar information for the metaverse, and to generate digital assets based on, for instance, a person's brain efficiency, resilience, or cognitive profile.

In some embodiments, DMDT data and DARWIN models are used to generate assets and content for the development of video games, where, for instance, specific brain and cognitive features of a player are reflected in an avatar, or specific game dynamics of a game are modulated by individual DMDT characteristics and real-time streaming of data via PERCEPTRON.

In some embodiments, DMDT data and DARWIN models (e.g., SYNAPSE, IMPROVE, OPTI-BRAIN) are used to create tailored, optimal, and/or improved in-game training strategies and learning protocols to enhance gaming performance.

In some embodiments, DMDT data and DARWIN models (e.g., SYNAPSE, IMPROVE, OPTI-BRAIN) are used to track brain health via PERCEPTRON, and identify tailored, optimal, and/or improved strategies for improving an individual's brain health.

In some embodiments, DMDT data and DARWIN models are used to create VR applications to enhance brain plasticity via a Virtual Augmented Environment (VAE) where specific combinations of stimuli and their features are presented to an individual to elicit a neuroplastic effect in the brain via modulation of the PNN and plasticity circuitry, with impact on learning, cognition and rehabilitation protocols.

In some embodiments, DMDT data and DARWIN models are used to guide STIMOLA protocols delivered in VR, to enhance the sense of embodiment, reduce motion sickness, and/or boost the effect of cognitive interventions delivered in a virtual environment.

In some embodiments, DMDT data and DARWIN models may be used for planning of personalized psychotherapy interventions, where the sequence of cognitive and behavioral exercises/tasks is defined on the basis of an optimal evolvability path for each individual. Methods and algorithms from OPTI-BRAIN, OPTI-COG, NEUROCREATOR and STIMOLA may be used to identify interventions acting directly on brain activity to be interleaved with cognitive and behavioral exercises in order to improve or maximize the impact of therapy.

In some embodiments, OPTI-BRAIN may perform simulations on DMDT data to identify brain targets for noninvasive brain stimulation therapeutic interventions in patients with neurological and psychiatric conditions.

In some embodiments, OPTI-BRAIN may be configured to perform simulations on DMDT data to identify brain targets for noninvasive brain stimulation interventions to boost plasticity via sleep modulation.

In some embodiments, OPTI-BRAIN may be configured to perform simulations on DMDT data to identify brain targets for noninvasive brain stimulation interventions to slow down or arrest brain tumor migration and recurrence.

In some embodiments, OPTI-BRAIN may be configured to perform simulations on DMDT data to identify brain regions affected by brain tumor recurrence, with the information being used to guide surgery.

In some embodiments, DMDT data and DARWIN models may be used to inform a general A.I. agent based on an individual's brain and cognitive properties via NEURO A.I.; the agent may be trained based on similar DMDTs from multiple individuals to generate a population-average behavior to be used for specific problem solving and decision making processes.

### BRAINPRINT

Some embodiments relate to solutions for capturing individual features of a given brain and combining the individual features with one or more of cognitive, behavioral and biometric data to provide a comprehensive summary of a given individual's abilities and potential, as well as to design personalized interventions to, at least in part, enhance cognitive function and brain health, optimize brain function, boost neuroplasticity and/or accelerate learning. The combined information may form a Dynamic Multilayer Digital Twin (DMDT) for the individual, representing a snapshot of an individual's profile at a specific moment in time and its temporal evolution as observed in the data as well as predicted by DARWIN models. The DMDT may represent a digital replica of local and distributed brain dynamics constrained by structural information derived from brain data. Mechanics of brain activity are described by equations and assemblies of equations describing local and distributed activity over time.

Some embodiments relate to creating a DMDT based, at least in part, on passive data, evoked data, active data, or any combination thereof. For example, the passive data may be obtained by recording passive data from the brain using noninvasive electrophysiology in the form of electroencephalography (EEG), and the dynamics of brain activity may be summarized in a series of indices and metrics describing topology, activity and dynamic of a given brain under the name of “Electrome” per its similarity to the brain Connectome.

The evoked data may be obtained by, for example, recording active data from the brain including, but not limited to, electrical and/or hemodynamic signals measured in response to external stimuli (e.g., in response to a flashing light or sound) or manipulation of brain state via specific instructions (e.g., induction of a meditative state).

The evoked data may be obtained by, for example, recording active data from the brain including, but not limited to, electrical and hemodynamic signals measured in response to cognitive stimulation as described as part of a cognitive assessment procedure where tests and tasks are administered to infer information about an individual's cognitive profile. Procedures may also include measuring the response to a cognitive intervention aimed at enhancing or restoring cognitive function in healthy individuals or individuals with a neurological or psychiatric condition.

The evoked data may be obtained by, for example, recording active data from the brain, including but not limited to electrical and hemodynamic signals measured in response to a psychological assessment procedure where tests and tasks are administered to infer information about an individual's psychological profile. Procedures may also include measuring the response to a psychological intervention aimed at providing psychological relief and/or alleviate symptoms of a particular psychological cognition including those related to mood and anxiety disorders.

The evoked data may be obtained by, for example, recording active data from the brain, including but not limited to electrical and hemodynamic signals measured during the completion of a task as part of a videogame. Brain activity related to specific aspects of a videogaming experience may be recorded in order to infer the neural correlates of in-game performance. In a similar embodiment, data are collected during a physical performance, such as a sport-related activity or during a meditation session.

The evoked data may be obtained by, for example, recording data from the brain, including but not limited to electrical and hemodynamic signals measured in response to, but not limited to, external electrical, magnetic or ultrasound perturbation of the brain performed using an apparatus mounted on an individual's head. Brain activity and corresponding cognitive and behavioral data may be collected before, during, and after the perturbation, to derive metrics of the individual's response to targeted noninvasive stimulation of the brain.

In some embodiments, brain perturbation is delivered via methods and techniques of noninvasive brain stimulation (NIBS) including, but not limited to, transcranial magnetic stimulation (TMS) in the form of, but not limited to, repetitive TMS (rTMS), patterned rTMS protocols such as Theta Burst stimulation, multi-pulse TMS and paired associative stimulation; transcranial electrical stimulation (tES) in the form of, but not limited to, transcranial direct current stimulation (tDCS), transcranial alternating current stimulation (tACS), and transcranial random noise stimulation (tRNS); focused ultrasound (FUS). The description of the STIMOLA module below provides additional details on methods and approaches for providing stimulation in accordance with some embodiments.

For any of, and not limited to, the modalities and applications described above, a DMDT may be created at least in part by examining properties of brain activity recorded at meso-and macro-scale levels, locally and across multiple brain structures, circuits and networks. The resulting model may be a digital replica of meso-macro scale dynamics of an individual brain, where information at different spatial and temporal scales is summarized at multiple levels including, but not limited to, a (i) structural level including information about brain wiring and local tissue properties, a (ii) functional level describing spontaneous and/or evoked activity measured in brain regions or local circuits, a (iii) dynamics level where multi-regional activity happening over time is summarized (for instance, functional connectivity and spectral coherence data across brain regions or networks), and a (iv) mechanistic level where algorithmic modes explaining and predicting functional and dynamics levels data are stored. Models include mathematical models accounting for layer-to-layer grey matter activity in the brain to those explaining long-distance network-level activity, such as that captured by high-resolution EEG. Machine learning and/or deep learning models may be used to validate predictions against null distributions and/or a template of random brain activity. Information from different data modalities may be summarized with metrics applicable to different data types, for instance those related to network-level activity as summarized by graph theory and network control theory metrics. Anatomical and functional templates may be used to ensure symmetry between data collected across modalities and different spatial resolutions (for instance, EEG and MRI). Metrics related to local activity may also be extracted and integrated with network-level metrics. Metrics related to activity may be applied to both static and dynamic data (e.g., time series data), as a summary of activity over time and/or a continuous discrete measure of activity over time.

For example, in some embodiments, a DMDT may be obtained at least in part by looking at the synchronicity between the activity of two or more brain regions, using metrics of static (functional), dynamic and effective connectivity. In some embodiments, a DMDT may be obtained at least in part by looking at the organization of brain networks, using metrics derived from graph theory, network topology analysis and network control theory. In some embodiments, a DMDT may be obtained at least in part by looking at patterns of eye movement recorded via a camera during the execution of one or more cognitive tasks. In some embodiments, a DMDT may be used to define a quantitative template of “healthy” brain and cognitive dynamics to be used as a reference for DARWIN applications involving, but not limited to, estimating brain potential, executing a brain optimization program via OPTI-BRAIN or defining therapeutic interventions.

Data may be assimilated and harmonized across data input modalities and data formats, including input from, but not limited to, EEG and neuroimaging data, cognitive metrics and hearth-rate variability. Subsequent analysis of individual data may be performed within a module (e.g., OPTI-BRAIN) able to summarize an individual's data into performance indexes divided in macro domains, including but not limited to a (i) Brain Performance Index, a (ii) Cognitive Performance Index, a (iii) Perturbation Index, a (iv) Learning Index, a (v) Plasticity Index. In some embodiments, a DMDT may be used to calculate a series of indexes capturing brain, cognitive and behavioral performance.

A Brain Performance Index may be derived from individual data, representing a person's brain capacity to efficiently propagate information within brain regions and networks, properly and dynamically allocate metabolic resources for different tasks.

A Perturbation Index may be derived from individual data, representing a person's brain capacity to respond to endogenous and exogenous perturbations in the form of, but not limited to, cognitive stimulation, electro-magnetic stimulation, behavioral interventions such as psychotherapy or meditation, or drugs.

A Plasticity Index may be derived from individual data, representing a person's brain capacity to, but not limited to, adapt to novel stimuli by creating novel brain connection or rearranging existing ones at the micro-meso-macroscale.

A Learning Index may be derived from individual data, representing a person's capacity to, but not limited to, generate, organize and apply novel knowledge through information acquisition and manipulation of novel inputs, including one's strategic ability to learn e.g., Learning to Learn).

In some embodiments, DMDTs from multiple individuals are stored in a database, and the stored DMDTs are used to estimate Brain-modes representing specific brain configurations used to build neuromorphic artificial intelligence (A.I.) algorithms; for instance, the average of DMDTs collected in individuals with a high IQ may be used to generate a general purpose A.I. module; DMDTs collected in individuals with high creativity may be used to generate a divergent thinking A.I. module; DMDTs collected in individuals with high executive functions may be used to generate a decision making A.I. module; DMDTs collected in individuals with a high Brain Performance index may be used to generate a backbone A.I. infrastructure where modules are connected and synchronized.

In some embodiments, DMDTs from multiple individuals are stored in a database, and the stored DMDTs are used to estimate Brain-modes representing specific brain configurations suitable for specific tasks; for instance, the average of DMDTs collected in individuals with a high learning index may be used to identify brain targets for neuromodulation interventions aimed at boosting learning and memory performance in individuals with a low learning index.

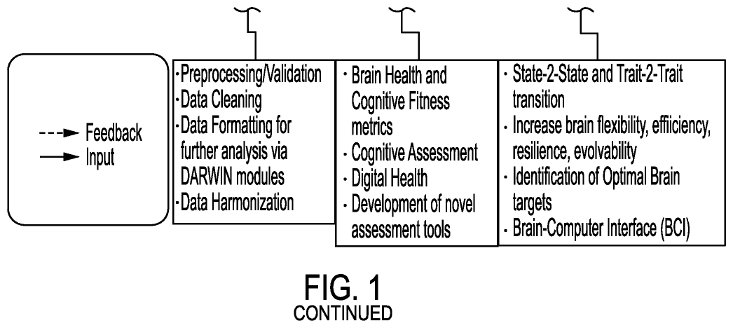

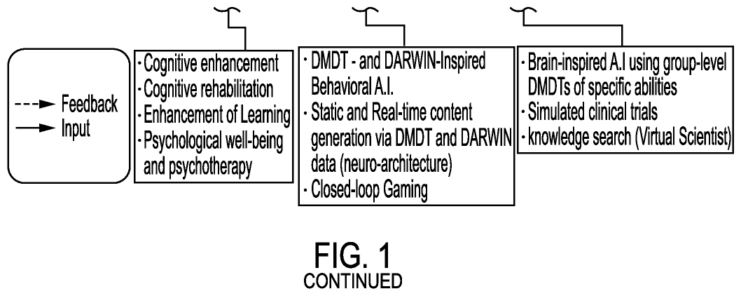

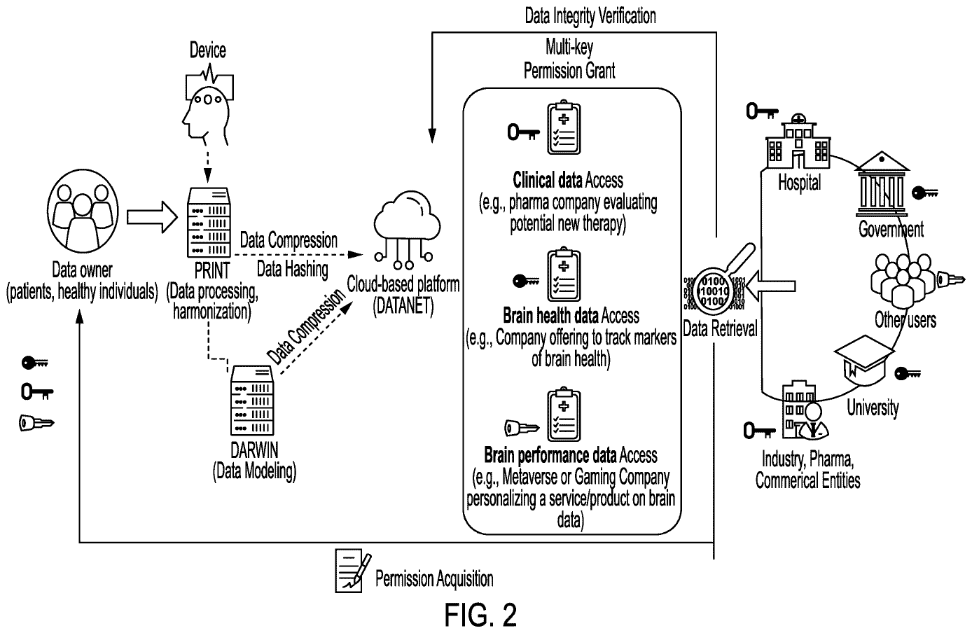

DMDT data may be stored at multiple levels for distinct and/or overlapping purposes. For instance, DMDT data may at least in part be locally stored in a PERCEPTRON device used to collect data as part of the DMDT creation process. The device may be configured to store data and host local routines for data harmonization and analysis, including at least some of those described in DARWIN. As shown in FIG. 2, DMDT data may at least in part be stored in a database DATANET (e.g., a cloud-based biobank), where DMDT data from multiple individuals is analyzed and harmonized to create, for instance, population-based DMDTs of brain health, cognitive performance and resilience to pathology, as well as identify candidate brain targets for neuromodulation interventions.

FIG. 2 shows an example cloud computing and blockchain architecture for data storage, processing, verification and distribution sharing across multiple platforms in accordance with some embodiments, as well as different scenarios involving different users and clients requesting access to data.

### Non-fungible Token (NFT) Generation and Use

In some embodiments, a DMDT is stored as a Non-Fungible Token (NFT), in the form of a digital packet containing information on an individual's brain structural and functional properties. In one instance, the NFT may be related to global characteristics of a given brain, and may be unrelated to a particular state or trait. In another instance, the NFT may be related to one or more specific characteristics of a given brain recorded during a particular state or moment in time, for instance during the successful completion of a cognitive task (e.g., successful memory encoding and retrieval), a behavioral task (e.g., meditative state, music playing), during a state of emotional activation (e.g., during retrieval of past traumas or during experiencing a trauma), during a state of high creativity or abstract reasoning (e.g., during the conception of the solution to a problem or during novel idea generation). The NFT may be then used as a template to derive DARWIN solutions to transition from a current DMDT to a past brain state/trait using approaches and methods from STIMOLA (e.g., using brain stimulation to re-enter a brain state of high creativity).

In some embodiments, a NFT may be used in clinical settings as a template to derive DARWIN solutions to transition from a current BRAINPRINT of a patient to a past brain state/trait using STIMOLA and including interventions including, but not limited to, transcranial electrical and magnetic stimulation. Applications may include, but are not limited to, using a preclinical DMDT (e.g., before onset of symptoms or detection of pathology biomarkers) of a patient to optimize neuromodulation interventions [STIMOLA] in an effort to ameliorate clinical symptoms or modify the disease course (e.g., use a DMDT of a patient with dementia collected before memory deficit onset, to optimize current brain processing towards effective memory encoding and retrieval patterns; use a DMDT of a patient with PTSD collected before the PTSD-inducing trauma, to identify patterns of change in brain activity and derive helpful interventions via DARWIN).

In some embodiments, a NFT may be used in human performance applications, as a template to derive DARWIN solutions to transition from a current DMDT of an individual to a past brain state/trait using STIMOLA. Applications may include, but are not limited to, using a DMDT of an athlete collected during peak condition and/or during a peak performance, to optimize neuromodulation interventions to re-enter a state of high performance; or use a DMDT collected during a deep meditative state to guide step-by-step modifications of brain dynamics needed to reach such state.

In some embodiments, a NFT may be enriched with data related to, but not limited to, psychosomatic response (e.g., stress response, galvanic skin response), cardiac activity (e.g., heart rate, heart rate variability), behavioral patterns (e.g., sleep activity and efficiency), cognitive status (e.g., memory performance, attentional levels, intelligence quotient—IQ), to further personalize the creation of a DMDT and define parameters for brain optimization. In some embodiments, a group of NFTs from individuals with similar profiles may be used to create group-level representations of brain and cognitive activity representing a particular group of individuals.

In some embodiments, patterns of brain activity recorded during a specific moment in time of particular relevance for an individual may be stored as an NFT for its value as a memory. In some embodiments, patterns of brain activity related to a specific moment in time during a videogame performance of particular relevance for an individual may be stored as an NFT for its replay value, its value as a template of a memorable performance, its value as training material to improve performance and train other individuals on in-game dynamics and performance.

An NFT may be represented by brain data alone, brain data combined with behavioral data capturing one or more performance characteristics, brain data combined with cognitive data, or brain data combined with other physiological data. In some embodiments, an NFT may include the both the data and the procedures for its generation, including data processing techniques and data visualization solutions.

In some embodiments, an NFT may contain a set of brain states collected via PERCEPTRON or a similar device, in the form of brain, cognitive and physiological data. The NFT is labeled by an individual as a specific memory of unique value, and the NFT is then used as a template to recreate the pattern of brain activity generating such memory. Memory re-creation may be achieved using DARWIN algorithms suggesting optimal connectome modulation approaches and tools from STIMOLA. NFTs for unique memories, moments and brain states may be manually generated by an individual by assigning labels to specific moments in time, or automatically generated via algorithms based on thresholds of brain, psychological and physiological activation marking the beginning and end of a uniquely valuable moment in time to be stored as an NFT. For instance, an NFT may be automatically generated during a high performance videogaming tournament to memorialize brain patterns supporting peak performance, or those happening during a celebratory event. An NFT may be automatically generated in moments of high emotional activation (for instance, when meeting a partner or member of the family after a long separation, when interacting with a pet or a child, when remembering a positive or negative memory). Brain state detection, analysis and storage may be performed via algorithms from DARWIN and stored in DATANET.

As part of DATANET, individuals may maintain control of an NFT generated by them and/or on the basis of their brain, physiological and cognitive data. Individuals may have the possibility of sharing their NFTs, editing and modifying their NFTs, exchanging and selling their NFTs using DATANET or other associated platforms.

In general, models of brain activity, behavior, cognitive performance, psychological profile and physiological profile collected as part of a DMDT and/or analyzed via DARWIN, may be stored as an NFT representing a specific, unique characteristic of a given individual. DARWIN models obtained by extracting features from DMDT of an individual may constitute a unique set of information describing an individual's phenotype related to one or more of DARWIN's indexes (examples of which are described herein), including but not limited to indexes of brain and psychological resilience, flexibility and evolvability. The characteristics of a given brain providing, for instance, a high state of evolvability and plasticity in a given individual may constitute an NFT uniquely identifying the individual whose data was used to generate such brain features. In some embodiments, the data from multiple individuals and DMDTs, collected in DATANET, may be used to generate group-level NFTs representing unique configurations of brain activity describing a specific group of individuals, for instance a group of patients with similar clinical characteristics or a group of healthy individuals with high working memory performance.

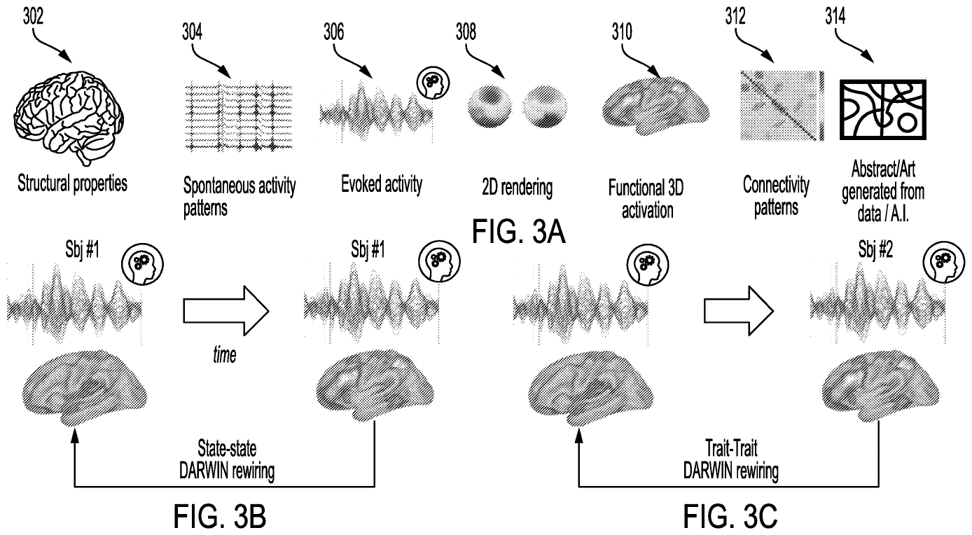

FIGS. 3A-3C schematically show multi-layer NFT creation from neural data in accordance with some embodiments. FIG. 3A shows a NFT may represent a structural property of a brain 302, its spontaneous activity patterns 304 unique to each individual, patterns of evoked activity 306 induced by various types of perturbation (including but not limited to electrical stimulation; magnetic stimulation; sensory stimulation as in the case of visual and auditory stimulation; an emotional stimuli evoking a psychosomatic response), a graphical representation 308 of said activity in 2D, a 3D rendering 310 of brain activity using high-resolution brain data or a 3D template, patterns of connectivity 312 and network-level activity characterizing how brain regions/networks/circuits interacts over time, and/or art 314 generated from brain data via generative algorithms, A.I. or human-guided procedures. FIG. 3B shows evoked activity may be recorded in an individual while solving a cognitive task or performing a particular activity (for instance, playing a videogame or solving a math problem); changes in brain activity might occur over time due to normal aging processes, lack of training and other factors; the individual may want to restore the previous pattern of brain activity in order to regain his/her performance; the NFT used at a first time point may be used as a template to guide rewiring of brain activity patterns using DARWIN. FIG. 3C shows that a similar procedure may be implemented to rewire brain activity of an individual to match that of a different individual with desirable patterns of brain activity and corresponding behavior/performance.

### SYSTEMS & HARDWARE [PERCEPTRON]

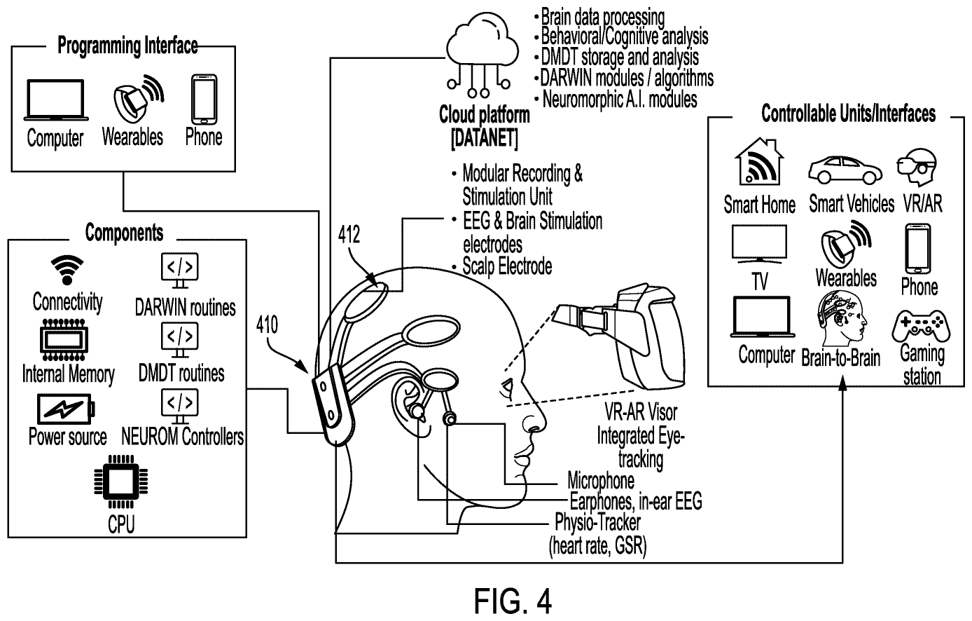

Some embodiments relate to a system for capturing information about an individual (referred to herein as “PERCEPTRON”). For example, FIG. 4 shows that the PERCEPTRON system may include, but is not limited to, a portable, wearable headset configured to record brain signals (e.g., EEG, blood flow), voice via a microphone, galvanic skin response (GSR) or similar data for stress-related responses, and/or heart/cardiac data (e.g., heart rate, heart rate variability), and to deliver a stimulation protocol received from STIMOLA.

In some embodiments, PERCEPTRON is a battery-powered headset 410 with recording electroencephalography (EEG) electrodes 412 arranged to be placed in contact with the scalp of an individual. The electrodes may be arranged to cover the entire scalp according to pre-specified locations depending on the data being collected. The PERCEPTRON system may have a modular structure with a main frame hosting electrodes and a power source (e.g., a battery), and a set of additional components that can be attached to the frame to expand the number of electrodes on the scalp, as well as other components including, but not limited to, a microphone, a VR headset or AR goggles. The PERCEPTRON system may include a processing unit (e.g., embedded in the frame) with computational capacity and a memory unit for local storage of data. The PERCEPTRON system may have wireless communication capabilities, including, but not limited to, circuitry that enables communication via Wi-Fi and/or Bluetooth. The PERCEPTRON system may be configured to connect directly with external hardware including, but not limited to, a smartphone, desktop computer, laptop, tablet, TV. The PERCEPTRON system may have connectivity capabilities allowing direct network (e.g., Internet) connection without the need of external devices; this capability may be used for, but is not limited to, cloud-based computing, real-time storage and processing of data, or interaction with other PERCEPTRON systems. Interaction with other devices allows for directly receiving and sending information about stored or real-time data from a given PERCEPTRON system including, but not limited to, EEG activity, heart-rate, and GSR. Connectivity capabilities may be used to interact with hardware other than PERCEPTRON systems via one or more Brain Computer Interface (BCI) applications including, but not limited to, controlling a TV set, a laptop computer, a mobile phone, and for more direct interactions with content such as controlling an avatar in a videogame or a metaverse application.

In some embodiments, the PERCEPTRON system also includes a battery-powered neuromodulation unit allowing for various forms of transcranial electrical stimulation (tES) via the same EEG electrodes included in the PERCEPTRON system. Neuromodulation also includes, for example, transcranial focused ultrasound (tFUS) and near-infrared spectroscopy.

In some embodiments, the PERCEPTRON system includes composite electrodes made of carbon nanotubes allowing for dry recording and dry brain stimulation without the requirement of gel application on the scalp. The carbon nanotubes may be positioned as conductive filaments inside a fabric cap positioned on the scalp. The filaments may be positioned across the entire scalp, and sub-portions of each filament may be activated by microcontrollers able to activate millimeter-resolution segments of the filament; this solution allows high-spatial resolution for both brain recording and brain stimulation, allowing a 1-1 match between observed brain data on the scalp and injected brain stimulation.

In some embodiments, the PERCEPTRON system includes electrodes for gel-based brain recording and brain stimulation equipped with a pressure-based dispenser system and active-impedance monitoring system; the gel may be stored inside the electrode and released when an impedance level evaluated on the surface of the electrode touching the scalp reaches a pre-specified threshold level. The system allows for minimal gel usage, avoiding bridge effects and signal contamination.

In some embodiments, the PERCEPTRON system is controlled via a multi-platform software package available for personal computing (PC) devices and/or mobile devices in the form of an app.

In some embodiments, the PERCEPTRON system may be used to acquire data for a DMDT of an individual and/or may be subsequently used for longitudinal assessments for the individual over time. Baseline and longitudinal assessments may be completed during resting-state activity and/or during execution of a particular task to evaluate evoked brain activity.

In some embodiments, the PERCEPTRON system may capture data used to create a DMDT, as described above. For example, an individual may be exposed to a VR or AR environment where behavioral and cognitive responses are captured using the PERCEPTRON system, with biometrics being recorded via a wearable data collection unit. The physical features of the VR/AR environment may be manipulated to elicit a target response and provide meaningful data for the creation of a DMDT. VR and AR may be used to overcome limitations of standard cognitive and behavioral assessment, as well as to administer special, non-ordinary stimuli tailored to probe brain and mental function (e.g., expose patients to enriched environments and non-ordinary stimulation patterns including sensory, auditory and visual stimulation).

In some embodiments, the PERCEPTRON system may be connected and/or controlled via a mobile device (e.g., smartphone, tablet), for remote monitoring and/or real-time data collection during daily activities, execution of particular tasks, etc.

In some embodiments, stimuli presentation and response recording using the PERCEPTRON system may be performed in a dedicated physical space allowing for more immersive sensory and cognitive stimulation, as well as exposure to more complex stimuli. As an example, the physical space may be a room that includes tools for audio and video stimuli presentation and recording sensors installed on each wall of the room. Sensors and stimuli presentation apparatus of the physical space may be connected with wearable devices used to guide or update a pattern of perturbations provided to the individual in real-time based on individual responses.

In some embodiments, the PERCEPTRON system may be configured to deliver noninvasive brain stimulation including, but not limited to, transcranial electrical stimulation. Stimulation may be delivered before, concurrently and/or after brain data collection.

In some embodiments, the PERCEPTRON system may be used for gaming applications, to monitor, guide/modulate brain and biometric data during gaming performance, and/or to extract DMDT data for the generation of gaming content and assets. In some embodiments, the PERCEPTRON system may be used as a digital biometric assessment tool to monitor activities of daily living and everyday cognitive and brain activity in healthy individuals. Data collected via the PERCEPTRON system and metrics extracted via DARWIN may be used to trigger in-depth assessments carried out in clinical settings, as in the case of an individual showing altered brain oscillatory activity at a weekly home-based assessment being invited for a clinical EEG assessment at a local hospital.

In some embodiments, one or multiple PERCEPTRON systems may be used to monitor brain activity from multiple individuals simultaneously, providing data for analysis of brain-to-brain communication relevant for applications in the field of decision making and cognitive neuroscience.

In some embodiments, the PERCEPTRON system may be used for clinical assessment in individuals with a neurological or psychiatric condition, as well as other medical conditions affecting the central nervous system.

### DATA PROCESSING AND ANALYSIS [PREPARE]

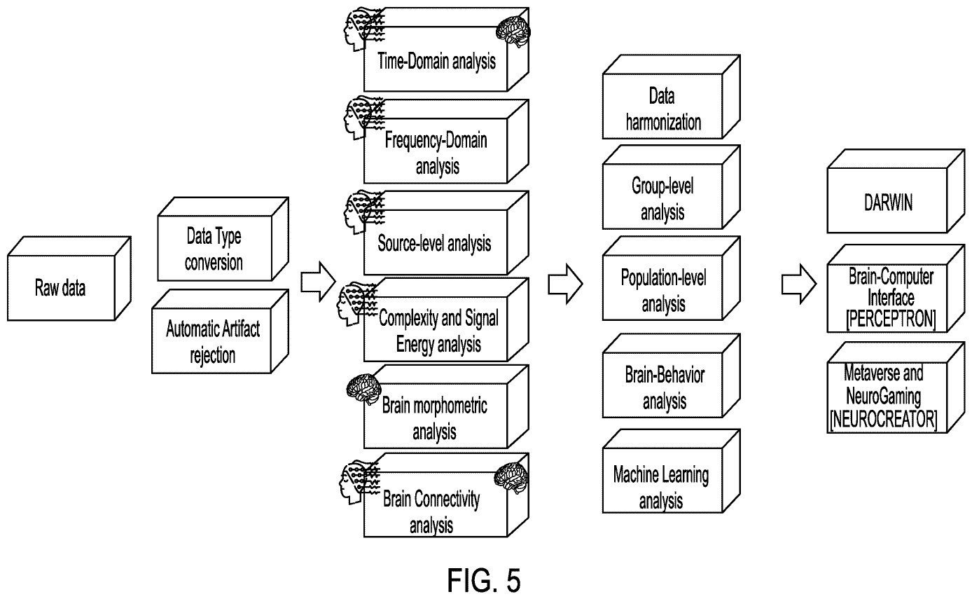

Information collected as part of BRAINPRINT and composing an individual's DMDT, may be processed and analyzed as part of a module PREPARE. Data cleaning, preprocessing, harmonization, and analysis methods may be implemented to transform raw data collected from devices into data suitable for data analysis and visualization. PREPARE includes pipelines for data transformation that facilitate, for instance, second-level analyses performed via the DARWIN module, brain-computer interface (BCI) applications informing brain stimulation interventions (e.g., via STIMOLA and/or PERCEPTRON), applications involving VR, or the metaverse and neurogaming (e.g., NEUROCREATOR). Pipelines may apply to multiple data formats and modalities including, but not limited to, brain scans, electroencephalography data, and behavioral data. In some embodiments, pipelines are used for analysis of different brain states, including analysis of spontaneous brain data and behavior, analysis of evoked activity in response to external or internal events/stimuli, analysis of brain data in response to external perturbation as in the form of transcranial electrical, magnetic or ultrasound stimulation. Pipelines for data preprocessing and analysis may include both unsupervised algorithms for automated processing and semi-automated, supervised algorithm for data interpretation and decision making. FIG. 5 illustrates an example data analysis pipeline including data processing components that may be included in PREPARE in accordance with some embodiments.

### Data Processing Methods

Data processing includes solutions for automated and semi-automated cleaning/preprocessing of data, with the possibility for manual identification of artifacts. Processing and analysis may be done as part of separate modules, covering, for example, (i) data collection, (ii) data validation and format conversion, (iii) data cleaning and preprocessing, (iv) data harmonization, (v) data analysis, and (vi) detailed report creation (including optimal stimulation target/parameters and summary of processing steps).

### Brain Scans Processing

When brain scans are available for stimulation target selection, two types of information may be used: (i) structural properties of the brain, including but not limited to density/volume/thickness/gyrification/sulcal depth of grey/white matter, CSF distribution, diffusivity and anisotropy of white matter, and spectroscopy profile of neurotransmitters; and (ii) functional properties of the brain including, but not limited to, hemodynamic response, blood perfusion, metabolic activity (e.g., glucose consumption), neuroinflammation levels, and protein burden. Steps for preparing the brain scans for statistical analysis include, but are not limited to, conversion of single images to a 3D volume format; segmentation in brain tissue classes; spatial and temporal filtering; removal of physiological noise; removal of image artifacts; extraction of average values and/or timeseries of brain activity, co-registration to a common anatomical or functional template for group-level analysis; calculation of evoked activity when multiple scanning conditions are present as in the case of block-fMRI data. Follow-up analysis may be performed on both voxel-based volumetric data or vertex-based surface images and includes masking of clean data on the basis of anatomical or functional atlases describing relevant networks or brain regions.

### Electrophysiological Data Processing

Data collected via electroencephalography (EEG) are often contaminated with recording artifacts including, but not limited to, eye blinks, heart rate, motion-related artifacts. The systems and methods described herein provide for data cleaning, preprocessing, and/or harmonization to at least partially reduce or eliminate the effect of such artifacts.

Preprocessing and cleaning steps include, but are not limited to: raw data conversion (e.g., in .edf format); trimming of raw data into epochs of predefined length; automated or semi-automated data inspection to identify EEG channels with excessive noise or artifacts; zero removal of muscle artifacts (e.g., according to voltage-based thresholds, kurtosis and joint probability); independent component analysis (ICA) to identify and remove components, with an additional data reduction via principal component analyses (PCA) to minimize overfitting and noise components; data interpolation; band pass filtering using a forward-backward filter; notch filtering to account for line noise; referencing to global average; a second ICA to manually remove all remaining artifactual components including eye movement/blink, muscle noise (EMG), single electrode noise, as well as auditory evoked artifacts (artifacts are identified and labeled on the basis of their spectral frequency profile, power spectrum, amplitude, scalp topography, and time course); application of machine learning and deep learning algorithms for identification of residual artifacts; interpolation of previously removed electrodes. Data cleaning and processing may be performed as a supervised method, e.g., with human validation of processing steps and visual inspection of data, or as an unsupervised procedure based on ML and AI with no human interaction.

### Concurrent Brain Stimulation and EEG Recording

In some embodiments, brain activity may be recorded during the delivery of noninvasive brain stimulation as described in the STIMOLA section. Unique adjustments to standard EEG processing pipelines may be used to ensure proper processing of stimulation-induced artifacts and interpretation of data.

In the example below, procedures for cleaning, preprocessing and analysis of EEG data collected before, during and after the delivery of TMS pulses are described. The same procedures may apply to concurrent tCS and/or focused ultrasound during EEG recording.

TMS-EEG analysis: In addition to typical artifacts present during EEG recording (e.g., eye movements and heartbeat), EEG data collected during TMS may be contaminated by TMS-specific artifacts including, but not limited to, a magnetic artifact changing the impedance of EEG electrodes, TMS-induced muscle artifacts characterized by high-frequency activity, and artifacts related to the TMS machine recharging process in-between TMS pulses. These artifacts usually have amplitudes several orders of magnitude bigger than EEG data, thereby confounding brain signals in the EEG.

Preprocessing and cleaning steps including, but are not limited to, raw data conversion (e.g., in. edf format); trimming of raw data into epochs of predefined length, including segments capturing pre-and post-TMS brain activity; normalization of post-TMS activity by subtracting the average signal amplitude of EEG data collected pre-TMS; automated or semi-automated data inspection to identify EEG channels with excessive noise or artifacts; zero padding of activity concurrent to the single TMS pulse to remove early signal decay and muscle artifacts induced by the TMS pulse (e.g., according to voltage-based thresholds, kurtosis and joint probability); independent component analysis (ICA) to identify and remove components including early TMS evoked high amplitude electrode, with an additional data reduction via principal component analyses (PCA) to minimize overfitting and noise components; interpolation of previously zero-padded signal across the TMS pulse; band pass filtering using a forward-backward filter typically between 1 to 150Hz; notch filtering to account for line noise; referencing to global average; a second ICA to manually remove all remaining artifactual components including eye movement/blink, muscle noise (EMG), single electrode noise, TMS evoked muscle activity, cardiac signal (EKG), as well as auditory evoked artifacts (artifacts are identified and labeled on the basis of their spectral frequency profile, power spectrum, amplitude, scalp topography, and time course); application of machine learning and deep learning algorithms for identification of residual artifacts; interpolation of previously removed electrodes.

Data cleaning and processing may be performed as a supervised method, e.g., with human validation of processing steps and visual inspection of data, or as an unsupervised procedure based on machine learning (ML) and A.I. with no human interaction.

### BRAIN ENHANCEMENT PLATFORM [DARWIN]

Once a DMDT is available, the DARWIN module may be configured to estimate the potential of an individual brain to grow, evolve, adapt and/or learn, in accordance with some embodiments. DARWIN may be configured to create a digital representation of an individual brain, including its structural and functional properties and corresponding cognitive and psychological hierarchies and abilities, allowing to perform simulations describing the response to stimuli such as normal and pathological aging, external perturbation as in the case of traumatic brain injury or brain tumors or magnetic/electrical stimulation, or psychological stressors such as trauma. For instance, DARWIN may be configured to identify optimal trajectories to guide brain change, both when the goal is a modifiable, transient target state, or a new stable brain configuration (a trait). DARWIN also allows to specifically modify one or more brain properties relevant for a given objective, for instance learning a new musical instrument or inducing a meditative state. Details on exemplary different applications of using DARWIN are described below. DARWIN may be informed by data collected via PERCEPTRON, by models and information from SYNAPSE and IMPROVE, may generate assessment tools via SCREEN, and/or guide procedures used in STIMOLA.

### DIGITAL BIOMETRICS [SCREEN]

Through SCREEN, DARWIN may be configured to generate data-driven digital biometric assessment tools and metrics. SCREEN may include methods for the generation of assessment tools quantifying cognitive, brain and psychological dimensions, constructs, and functions, promoting personalized digital biometrics of brain, cognitive and mental health. In embodiments, assessment tools are deployed in the form of digital tasks or games via portable devices including, but not limited to, mobile phones, tablets, laptops and wearables. The mechanics and design of assessment tools may be based on analysis of data collected as part of DMDTs, and informed by design principles and neurobiological/neurophysiological/neurological concepts and models from SYNAPSE and IMPROVE. Assessment tools may be based at least in part on knowledge related to brain plasticity and science of learning, in combination with original data related to the architecture of human cognition and psychological wellbeing of an individual (e.g., see IMPROVE).

In some embodiments, information obtained via OPTI-BRAIN and OPTI-COG may be used to design ad-hoc cognitive and brain health assessment tools based on individual qualities derived from brain and cognitive data. Information used for the creation of assessment tools may be related to a single individual or a group of individuals sharing a particular feature of interest, for instance, a similar performance for a cognitive function (e.g., high working memory capacity) or a neurological condition (e.g., patients with dementia). Brain characteristics related to said function or condition may be modeled either from spontaneous activity data or from activity evoked in response to external perturbation. In an example application, DMDT data from a sample of individuals with a diagnosis of dementia and memory problems may be aggregated via PREPARE, and features related to brain plasticity and resilience may be extracted via DARWIN. Altered plasticity mechanisms and low levels of brain resilience in particular areas of the brain may suggest that a cognitive test capturing these brain features may be valuable for diagnostic and prognostic purposes in individuals with dementia and memory problems. As a result, a set of stimuli may be combined in a specific sequence in order to conduct a stress-test on those brain regions with low plasticity and resilience, thus measuring brain resilience to perturbation.

In some embodiments, cognitive, brain and psychological assessments are delivered via a VR or AR platform, including in the form of gamified applications. In some embodiments, cognitive, brain and psychological assessments are conducted while recording biometric data via a device including, but not limited to, PERCEPTRON; individual performance at the assessment tasks may be linked to specific patterns of brain activity for the generation of evoked-brain activity metrics of cognitive, brain and psychological function and health.

In some embodiments, a perturbation-based assessment is performed where brain and physiological activity recorded in biometrics is modulated by external stimuli, generating perturbation-based biomarkers indexing an individual's brain/cognitive/psychological capacity to adapt to perturbation as a measure of plasticity.

In some embodiments, physiological data collected in combination with brain data may include, but is not limited to, EKG, GSR, eye movements via eye-tracking technology, and body temperature.

In some embodiments, assessment tasks are developed to index metrics calculated via DARWIN such as those described in the following sections including, but not limited to, metrics of brain potential, fitness, resilience, adaptability, evolvability, stability, and plasticity. SCREEN may be configured to generate assessment tasks for each metric and index created as part of the BRAINPRINT and DARWIN modules, and in combination with protocols included in STIMOLA.

In some embodiments, assessment of brain properties is conducted to derive recommendations on beneficial activities and interventions personalized for a given individual on the basis of their DMDT. Interventions and activities may include, but are not limited to, psychotherapy, cognitive training, brain stimulation protocols, meditation and physical exercise.

### BRAIN POTENTIAL ESTIMATION

Every brain is characterized by various elements composing its structural and functional connectome, including for instance the amount of grey matter in each brain region, the number and strength of white matter fibers connecting different regions and networks, the vasculature supplying nutrients to brain structures, as well as the functional organization of the interplay between brain elements (synchronization patterns, oscillatory dynamics, local coherence patterns). This complexity spans both the micro, meso and macroscale of the human brain, with many dynamics also taking place across scales. Each individual connectome, at any scale, is the result of genetic and environmental factors, with the latter being more related to early life experiences as well as sustained practice and learning activity gradually shaping brain structure and function over time. While functional connections and dynamics are typically considered the most versatile and flexible, e.g., more susceptible to change, structural connections tend to be more robust and less responsive to change, providing a “backbone” for brain activity and computational processes. A granular understanding of the structural and functional properties of the brain, as well as their interplay (e.g., as provided by a DMDT), may be important for understanding the potential for change of a given brain. The system and techniques described herein allow for computationally estimating the potential for change of a given brain on the basis of a series of tests and simulated manipulations of a given structural and functional connectome, resulting in a quantification of a series of indexes holding predictive power over narrow scopes (e.g., learning a new musical instrument, improving a certain behavior) and broader scopes (e.g., successful aging, overcome post-traumatic stress disorder, avoid delirium onset after elective surgery). The estimation process may include analysis of existing patterns of activity as well as connectivity, but also a quantification of potential new patterns of activity guided by structural and functional constraints of the present DMDT.

Some of the systems and methods described herein relate to approaches for Adaptive Systematic Rewiring (ASR) used to estimate multiple parameters and indexes. The indexes may including, but are not limited to, a brain evolvability index, a brain optimization index, a brain adaptability index, a brain stability index, a brain neuroinflammation index, or a brain associative plasticity index, each of which is described in further detail below.

### Brain Evolvability Index

Given, at least in part, the number, location and strength of brain connections, as well as magnitude and frequency of local activity in the brain, a set of constraints may be imposed over an evolutionary algorithm able to compute mutations (e.g., all possible mutations) of the original feature set until reaching full development of the resulting network where no additional manipulations are possible. Several metrics may be used to summarize the performance of a given brain including, but not limited to, the number of modifications the brain can sustain, the number of configurations it can reach, and the level of energy needed to reach each configuration.

In some embodiments, simulated evolution may be based on different targeting strategies including, but not limited to, random targeting, topology-based targeting, modularity-based targeting including separate simulation for intra-module and inter-module connections manipulation, or hierarchical targeting.

In some embodiments, simulated evolution is performed via a connectome rewiring process, where existing, active or silenced connections of a network made of brain data are modified. Modifications may include, but are not limited to, a change in the strength of a given connection, a change in its input and/or output, its reactivation, or its reallocation to a different target brain structure. A fixed amount of energy available for rewiring may be set on the basis of one or multiple parameters including, but not limited to, the amount of circulating blood-oxygen-level-dependent (BOLD) signal when considering functional MRI data, total available metabolic energy as estimated by techniques including PET or SPECT, vascular activity and perfusion levels. Given the finite amount of energy for rewiring, each connectome is exposed to a rewiring procedure where each modification is assigned a cost. As a result, each connectome (e.g., each individual brain of an individual, or each brain state), can undergo a finite amount of rewiring steps, thus determining the intrinsic potential of a given brain to rewire itself, modify and optimize its connectome and modulate its resulting behavior, cognition and performance. The number of possible rewiring steps and the overall potential of a connectome to be rewired based on energy constrains may constitute a “Rewiring Index.”

In some embodiments, simulated evolution may be based on a fixed amount of energy used to induce manipulations to the connectome, based on brain metabolic data and estimates of brain response to perturbation. In some embodiments, simulated evolution may be based on increasing or decreasing a given connectivity between brain regions, increasing or decreasing brain activity in a given brain region, increasing global connectedness of a brain region or network, or increasing or decreasing synchronicity between brain networks. The magnitude of the increase and decrease may be defined based on, but not limited to, brain perturbation and plasticity indexes.

In some embodiments, simulated evolution may be based on amplifying existing patterns of connectivity and local brain activity, reducing noise and improving on intrinsic characteristics of a given brain (e.g., magnifying individual DMDTs). In some embodiments, simulated evolution may be based on different levels of compensation of connectivity and activity based on past changes induced to the DMDT including, but not limited to, redistribution of functional connectivity to neighboring regions, redistribution to positively connected regions, redistribution to regions belonging to the network being manipulated, or redistribution to regions belonging to the module being manipulated.

In some embodiments, simulated evolution may be constrained by information on structural wiring of the brain, expressed as the density of white matter fibers, their anisotropy and mean diffusivity, and myelination levels. In some embodiments, simulated evolution may be constrained by information on local metabolic brain activity and perfusion levels, as measured via, but not limited to, PET imaging and arterial spin labeling (ASL) MRI.

### Brain Optimization Index

Given, at least in part, the number, location and strength of brain connections, as well as the magnitude and frequency of local activity in the brain, a target configuration representing an optimal functioning configuration for the human brain may be considered as a reference target towards which brain modifications may be made. The optimal configuration may include features related to network functioning such as, but not limited to, a state of high small-worldness (representing the optimal ratio between segregation and integration), or high network modularity. Some features of network functioning may be selected based on their association with favorable traits, such as, but not limited to, high intelligence quotient (IQ), high memory performance, or high rate of information processing. The brain optimization index may provide, at least in part, a distance metric between an individual current state and the most desirable state from a cognitive and computational point of view.

### Brain Adaptability Index

Given, at least in part, the number, location and strength of brain connections, as well as magnitude and frequency of local activity in the brain, a given brain is exposed to a series of state changes and its capacity for adapting to such changes may be quantified in a computational model taking into account the amount of energy required to adapt to state change and the duration of the induced adaptation. Brain adaptability may be used as a measure of neuroplasticity and network plasticity, including measures related to network memory of previous state changes and network generalization (e.g., a brain's ability to predict the broad category of upcoming changes given the history of past ones, leading to generalization of knowledge and enhanced preparedness for future change). State changes can be local or distributed. Brain adaptability may inform a brain's tendency to change over time and the magnitude of such change, to then constrain analyses related to state or trait change.

### Brain Stability Index

Given, at least in part, the number, location and strength of brain connections, as well as magnitude and frequency of local activity in the brain, a given brain is exposed to a series of state changes and its average distance from any possible state transition may be quantified. Brain stability may be used as a metric of brain current tendency to approach a change of its ongoing dynamics, as observed in the analysis of network criticality and avalanches.

### Brain Neuroinflammation Index

Given, at least in part, the number, location and strength of brain connections, as well as magnitude and frequency of local activity in the brain, an individual is exposed to a series of stimulation over two brain regions located at the receiving ends of a white matter brain tract. The delay between stimulation of one end, e.g., a first brain region, and generation of a brain response on the second end, e.g., a second brain region, may be computed and used as a proxy of conduction delay between the two target brain regions; given the relationship between conduction speed and myelination of white matter fibers in the human brain, the conduction delay may be computed multiple times over a given time window including seconds, minutes, hours, days, weeks, months and years. Longitudinal changes in the delay may be used as an index of altered signal propagation in the white matter fibers which may signal the presence of conditions including, but not limited to, altered myelination and presence of lesions. In some embodiments, brain inflammation levels are quantified via analysis of spontaneous and evoked brain activity recorded via noninvasive methods including, but not limited to, EEG and NIRS. Activity in a specific frequency band may be measured (e.g., between 1 Hz and 9 Hz), and related to activity in a high-frequency EEG band (e.g., between 19 Hz and 85 Hz). Activity may reflect both local and long-range, distributed connectivity in the brain, capturing speed of processing and fiber conductivity. An index, the electrical-neuroinflammation index (ENI²), is computed at both global level, by looking at oscillatory activity across the brain/scalp, and locally by selecting specific electrodes located in regions at specific distances across the scalp; in particular, the ratio between electrodes located in frontal and parietal areas vs. those located in occipital and temporal cortices is computed, as an index of cognitive vs. sensory processing streams capturing different speed of processing. Visual and auditory stimuli may be delivered to amplify the signal across regions; in some embodiments, a low-voltage alternating electrical stimuli is delivered to each electrode at a resonant frequency to elicit a local and distributed response; the propagation of signal is tracked across the selected electrodes (electrical tomography detection); amplitude and frequency of early and late potentials generated by the perturbation are combined together and inputted into the ENI² score of an individual. ENI² scores may be used to confirm or inform cognitive and neuroimaging evaluations investigating the presence of neurodegeneration and neuroinflammation in the brain and central nervous system of an individual.

### Brain Associative Plasticity Index

Given, at least in part, the number, location and strength of brain connections, as well as magnitude and frequency of local activity in the brain, two brain regions may be stimulated via two quasi-simultaneous magnetic or electrical stimuli in order to induce paired-associative plasticity. Repeated quasi-simultaneous stimulation generates long-term potential in each region and a process of Hebbian plasticity across the two brain regions. The rate at which the two regions display Hebbian plasticity is dependent from the state of the two regions and the integrity of the monosynaptic white matter fibers connecting the two regions. By measuring the rate of association over the course of a fixed number of stimulations, a dose-response curve may be obtained for any given pair of brain regions, in any given brain of any individual. Each curve represents the corresponding brain's ability to increase interregional connections and generate plasticity, and might serve as an index of altered brain plasticity in neurodegenerative conditions characterized by increased neuroinflammation or neurodegenerative processes such as dementia, Alzheimer's disease, or multiple sclerosis.

In some embodiments, after having obtained a detailed map of brain structure and function (e.g., as represented as a DMDT), the brain's potential to change (evolve) towards other configurations may be estimated. The estimate may be based on, for example, the number, location, strength, configuration of brain regions and connections, and a finite number of iterations (“changes”) a brain can sustain/support on the basis of its past and current behavior/activity.

In some embodiments, OPTI-BRAIN may be based on concepts from evolutionary biology, evolutionary game theory, network control theory, network theory, agent-based modeling, artificial intelligence (A.I.), heat diffusion models, and/or epidemiological spread models.

In some embodiments, estimation of a brain's potential may be used to identify optimal strategies for enhancement, by calculating a similarity index with specific brain states/traits, and identifying the hierarchy of brain modifications to be applied in order to reach a goal state/brain.

### Brain and Cognitive State and Trait Transitioning

Starting from the same DMDT, in some embodiments, DARWIN may be used for parametrization of the trajectory of transition from a present brain state or trait to a target brain state or trait, by simulating multiple modifications (e.g., every possible modification) of the structural and functional connectome, and deriving a sequence of events (e.g., an optimal sequence of events) represented, for example, as a change of a connection strength, reduction of local activity in a specific brain area, etc., allowing to transition from a current state/trait to a desired state/trait utilizing the least amount of energy and time.

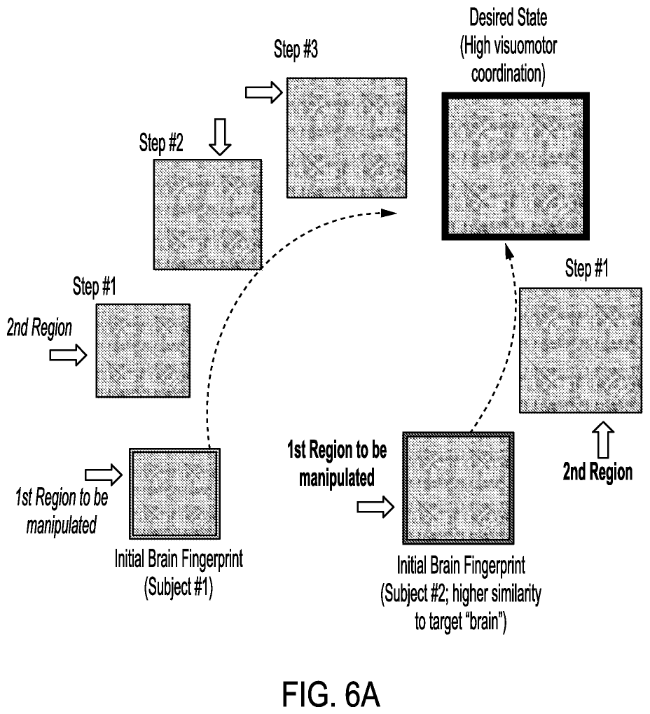

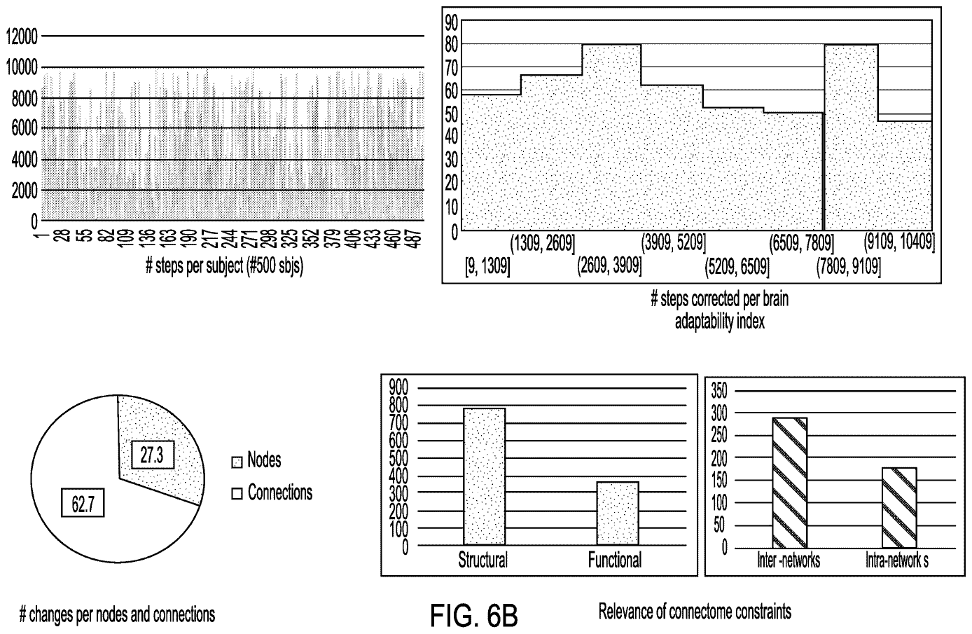

FIG. 6A shows an example of individual differences in state or trait transitioning, where two individuals aiming to reach the same target state follow two separate trajectories with a different number of steps required to evolve towards the desired state due to differences at baseline. FIG. 6B shows metrics of state transitioning within a sample of 500 subjects, highlighting individual differences in brain's potential for change.

In this framework, state transitioning is used as a term to indicate a more acute and transitory change of brain function, as well as a change in a given “brain state” (e.g., being in an optimal brain state to perform task X). When techniques able to induce long-lasting changes and promote brain plasticity such as those part of non-invasive brain stimulation methods are used, the application of Noninvasive Brain Stimulation (NIBS) techniques and their long-lasting brain changes are expected, and the effects can lead to trait transitioning, e.g., more durable changes of brain structure and/or function leading to changes in trait-level measures (e.g., intelligence, memory capacity, creativity).

In some embodiments, the estimated brain potential may be used to guide the planning of interventions (e.g., using STIMOLA, SYNAPSE, NEUROCREATOR) aimed at promoting brain transition in a specific direction (e.g., a target state or trait). Brain characteristics may be used to identify the most appropriate brain targets (e.g., regions, connections) to be modulated to reach a target state or a target trait.

In some embodiments, the target state may be defined as a state related to a particular cognitive activity (e.g., memory processing; meditative state). In some embodiments the target state may represent a prior state of the same individual recorded in the past, including but not limited to a brain state of successful memory encoding or retrieval for a patient suffering from dementia. In some embodiments, the target state may represent a specific brain state related to a high physical performance state.

In some embodiments, the target trait may be defined as a prior brain configuration of the same individual recorded in the past (e.g., brain configuration before dementia onset), that can be used to tailor a brain modifier and bring the brain back to a pre-disease onset state. In some embodiments, the target trait may be represented by a template trait representing a specific configuration linked to a cognitive trait (e.g., high IQ brain), or a cognitive mode related to a particular skillset (e.g., brain of a mechanical engineer). In some embodiments, the target state or trait may be defined as a prior brain configuration of the same individual stored as an NFT.

### BRAIN OPTIMIZATION

Changes of state or trait can be applied to modifications of brain activity that are not explicitly related to a measurable cognitive or behavioral ability, but rather a change in brain physiology itself with implications for brain health or disease. In some embodiments, through careful brain mapping and network analysis, each brain's resilience (e.g., a system's ability to absorb external attacks or internal failures without losing performance), adaptability (e.g., a system's ability to rapidly adapt to attacks and failures on the basis of its wiring/connectivity profile) and flexibility (e.g., a system's ability to shift from one given state to another without need to alter its structure, crucial for adaptability), may be quantified. DARWIN may be used to identify and plan targeted brain optimization (e.g., brain stimulation, cognitive programs, behavioral therapy) solutions in order to, e.g., (i) increase resilience to brain changes occurring during physiological and pathological aging, such as brain atrophy, loss of crucial network nodes, decrease in metabolism in regions relevant for memory performance; (ii) increase resilience to external events thereby decreasing the chance of stress-related pathological response (e.g. PTSD); (iii) optimize resilience and adaptability before general surgery to avoid onset of post-surgical delirium; (iv) increase brain resilience in conditions where the influence of external stimuli is not properly tolerated, such as psychiatric disorders and conditions related to high levels of anxiety and hypersensitivity; all these approaches and others fall under the concept of “brain shielding,” with other possible implications expanding into wellness and overall brain health.

In some embodiments, a DMDT is used to identify the optimal targets to increase a brain's ability to respond to external perturbation, e.g., brain resilience. The best brain targets (e.g., region, connection) and the sequence of changes needed to boost resilience may be identified as part of a Brain Shielding Protocol (BSP). Brain modifiers may then be used to modify a brain configuration through steps involving the modification of, at least in part, activity of a specific region(s), the strength of specific connections, and/or the organization of brain functional networks, according to principles defined in DARWIN. In an example, the DMDT of an individual preparing for a stressful activity, for instance a soldier preparing for deployment in a mission or a person with PTSD about to relive a traumatic experience, may be used to define a set of target locations in the brain whose manipulation may lead to an increase in brain resilience against perturbation. A specific sequence of actions including, but not limited to, the delivery of noninvasive brain stimulation to specific brain regions and brain connections over multiple sessions and the exposure to cognitive and sensory retraining protocols, including VR-based applications, may be defined according to a DARWIN resilience optimization algorithm. Subsequently, the individual may receive the treatment, and multiple measurements of brain resilience may be capture over time to assess the impact of the BSP, including those using PERCEPTRON. The duration, frequency and intensity of the BSP may be defined according to desired effects, based on expected duration of the effects (acute—minutes, short-lasting—hours, long-lasting—weeks-months).

In some embodiments, a BSP includes a protocol composed by exposing a given brain to progressive, adaptive, perturbations in the form of magnetic, electrical and ultrasound stimulations. Perturbations may be targeted towards nodes, regions, networks of the brain previously identified via a DMDT and DARWIN. Application of serial perturbation stimuli of modulated intensity induces a response of brain networks and circuits in a desired direction (e.g., by performing guided rewiring). An overall increase in the brain's resilience to external perturbation may be built via long-term potentiation (LTP) plasticity processes and network remodeling. In some embodiments, perturbations are delivered using sensory stimulation in order to elicit specific responses in sensory regions of the brain. Perturbations include visual, audio and tactile stimulation, with visual and audio perturbations being delivered via various media including VR and AR techniques.

In an example embodiment, a DMDT is used to identify the optimal targets to increase the brain's modularity, by modifying the strength, direction and/or temporal dynamics of specific connections between brain regions belonging to known brain networks and circuits. In an example embodiment, a DMDT is used to identify the optimal targets to increase the brain's flexibility by decreasing the strength of connections between brain networks responsible for redirecting attentional load during perception and networks responsible for cognitive processing including memory and inhibition functions.

In an example embodiment, DMDT and DARWIN indexes are used to determine a personalized dietary regime for an individual based on his/her profile of brain activity and notions related to the Gut-Brain axis, including but not limited to, their brain's resilience levels, modularity, flexibility, and level of neuroinflammation. The dietary regime may be determined by selecting nutrients and ad-hoc supplements associated with effects over brain activity including, but not limited to, vitamins, probiotics, minerals, and proteins. Personalized dietary interventions may be defined by looking at the effect of specific nutrients on the gut microbiome as well as on the brain, including but not limited to, anti-inflammatory effects, blood flow and perfusion effects, effects on intracranial pressure and effects on brain oscillatory patterns. In some embodiments, a specific dietary regimen is selected for its anti-aging properties including, but not limited to, Vitamin B1, B6, B12 and folate (B9), Potassium, calcium for its impact on brain plasticity, magnesium, and Beta-Carotene for its interaction with ApoE status in healthy individuals and patients with Alzheimer's disease.

In some embodiments, DMDT and DARWIN are used to personalize dietary information for an individual analyzing a brain's response to different nutrients and estimating the optimal set of nutrients to elicit a desired brain change including state-to-state and trait-to-trait transitions. In an example application, an individual's brain activity is recorded via PERCEPTRON before, during and after the ingestion of specific food and nutrients, and his/her brain activity is processed via PREPARE. The individual may also be exposed to systematic perturbation of brain, cognitive and sensory activity via SCREEN including, but not limited to, brain electrical stimulation, visual stimulation, and/or auditory stimulation. The brain's response to perturbation may be quantified and used to track the individual's brain and cognitive state over time in response to different nutrients and food products.

In some embodiments, the resulting brain and cognitive profile obtained in response to different nutrients and food products may be used to (i) identify a dietary plan able to optimize brain and cognitive performance, by increasing indexes of brain health and cognitive health identified in IMPROVE, OPTI-BRAIN and OPTI-COG. Longitudinal evaluations of brain and cognitive profile may be conducted over time to further optimize personalized dietary regiments. Example applications include, but are not limited to, the optimization of dietary regimes (i) to boost brain abilities and cognitive performance in healthy individuals, (ii) to accelerate recovery from injury, (iii) to accelerate post-surgical recovery by reducing neuroinflammation, (iv) to increase focus in special operators, and (v) to increase muscle growth and increase physical performance in athletes by looking at dietary regimes able to increase synchrony between brain activity and muscular response.

In some embodiments, the resulting brain and cognitive profile obtained in response to different nutrients and food products may be used to (i) identify existing nutrients and combinations of nutrients, or to (ii) design novel nutrients and combinations of nutrients, able to optimize brain and cognitive performance, by increasing indexes of brain health and cognitive health identified in IMPROVE, OPTI-BRAIN and OPTI-COG. Example applications include, but are not limited, to the design of nutrients and combination of nutrients (i) to boost brain abilities and cognitive performance in healthy individuals, (ii) to accelerate recovery from injury, (iii) to accelerate post-surgical recovery by reducing neuroinflammation, (iv) to increase focus in special operators, and (v) to increase muscle growth and increase physical performance in athletes by looking at dietary regimes able to increase synchrony between brain activity and muscular response.

### EXAMPLE USE CASES BASED ON EXPERIMENTAL DATA

Some embodiments of the system and methods described herein are further illustrated by the following examples and detailed protocols. However, the examples are merely intended to illustrate embodiments and are not to be construed to limit the scope of the technology described herein.

Brain optimization: increasing brain resilience to perturbation/stress Resilience is the ability of a complex system to retain its functional properties and performance in face of external perturbation, such as environmental factors like disease, stress, and aging. In case of the brain and pathology, resilience can be considered as the individual capacity of the brain to sustain a damage of great intensity and/or for a long time before the display of overt symptoms. The capacity to recruit additional regions to compensate for and/or defer disruptions and consequence is one of the potential factors able to increase resilience in the brain. Intrinsic properties of the structural wiring of the brain, as well as its functional organization, may be linked to different levels of resilience. The possibility to map an individual brain's resilience, identify candidate target regions/networks/system for its enhancement, and artificially manipulate resilience levels via external modulation tools, represents an approach with repercussions for both brain health and brain pathology. Such an approach may also allow the development of novel cognitive enhancement applications, clinical therapeutic tools, or solutions to enhance brain health and overall wellness improvement techniques. The data reported below show an example of the impact of targeted, personalized noninvasive brain stimulation on brain resilience in healthy individuals, demonstrating the application of BRAINPRINT, DARWIN and STIMOLA (within an Adaptive Systematic Rewiring framework) to change the brain's ability to respond to external perturbation.

A DMDT was created for a sample of healthy controls. DARWIN was used to estimate the optimal modulation targets to boost brain resilience based on simulated lesioning of each possible connection and node of the brain network for each individual and consequent estimation of induced change in brain performance under stress. Once the optimal target brain regions were identified, a session of Transcranial Magnetic Stimulation (TMS) was performed with concurrent EEG recording before, during and after each TMS pulse. EEG data were analyzed to examine brain efficiency and other measures indexing resilience to external perturbation. These measures included the largest connected component (LCC) corresponding to the largest set of nodes whose pairs are connected by at least an edge as the main outcome. A corresponding drop in the LCC was recorded as a measure of the induced damage. At each lesioning iteration, the nodes'degree was re-calculated, and the order of removal adjusted based on the effect of previous removal. Also included in the measures was the Speed of Decay, representing the individual pace of brain connectivity matrix lesioning that was computed as the slope of decay of the LCC following the targeted removal of edges; the Early Edges Drop resulting from the overall amount of connections that needed to be lost before the LCC would be destroyed; the Late Edges Drop corresponding to the overall amount of connections' loss necessary to completely destroy the LCC; and the Collapse point, or the point of maximum deflection in the lesioning curve of the LCC, based on the targeted removal of edges.

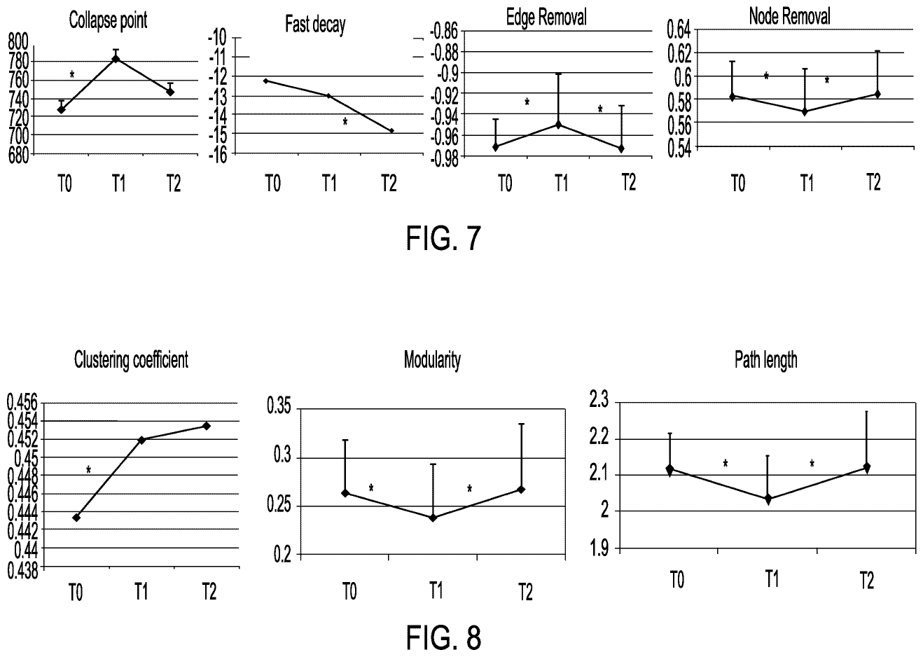

FIG. 7 shows that perturbation of the brain via STIMOLA induced significant increase in resilience measures across the entire brain (Note: *p<0.05). After targeted TMS, the whole brain network was more connected and more resilient, gradually returning to its original state after stimulation. This outcome is explained by both graph measures, with a decrease in the characteristic path length of the network after the TMS pulse, and resilience metrics, with a significant increase in the number of links needed to decrease network efficiency (Late Edges Drop) after TMS. Higher resilience, as was shown in this case, may be explained by a greater network stability, such as that both the point of maximum deflection and the point of complete network depletion occur at more advanced stages of lesioning. Repetitive application of resilience-boosting perturbation led to sustained states of heightened resilience outlasting the perturbation period, thereby contributing to increased resilience in non-experimental settings (e.g., daily living, stressful work environment).

In some embodiments, resilience boosting and brain shielding may be performed using techniques other than TMS, including but not limited to transcranial electrical stimulation. In some embodiments, resilience boosting and brain shielding may be performed via the delivery of cognitive perturbations in the form of visual, auditory, tactile and cognitive stimuli in a temporal sequence to activate and reinforce specific brain activity patterns linked to brain resilience.

In some embodiments, analysis of brain resilience and brain health are performed before surgery including, but not limited to, brain surgery, cardiac surgery and orthopedic surgery. Brain data may be collected via PERCEPTRON to build an individual DMDT via PRINT. Data in the DMDT include, but is not limited to, brain data collected via PERCEPTRON and Magnetic Resonance Imaging (MRI); cognitive data; medical history; perturbation-based biomarkers collected via PERCEPTRON while an individual was receiving sensory, electrical, magnetic stimulation. DMDT may be used to define the optimal brain state able to reduce post-surgery complications (e.g., delirium, cognitive deficits, neuroinflammation) and/or accelerate recovery via OPTI-BRAIN. Recovery may include, but is not limited to, physical rehabilitation, including assisted robotic rehabilitation, physical therapy, cognitive training and cognitive rehabilitation. In an example application, brain state analysis may be performed to quantify brain resilience and optimal brain state to undergo general anesthesia for hip replacement surgery; brain data may be collected via PERCEPTRON, cognitive data may be collected via SCREEN, data may be processed via PREPARE and a DMDT of the patient may be created. DARWIN may then be used to create a differential matrix between the patient's current pre-surgical brain and cognitive state, and the optimal state to maximize recovery; in an example application, the system estimates that high brain modularity and high segregation of the motor network including lower limbs representation are preferable to avoid hyper-connectivity which could lead to engagement of motor brain areas in non-motor functions during recovery.

In some embodiments, the assessment is repeated over time after surgery to create a longitudinal projection of recovery, which may be used to adjust training and rehabilitation protocols. OPTI-BRAIN and OPTI-COG may be used to maximize cognitive and motor recovery over time, with applications including, but not limited to, stroke recovery, motor rehabilitation after orthopedic surgery in athletes, and patients with brain tumors.

### Optimization of Brain Function and Brain Health

In this example, data showing the impact of targeted, personalized noninvasive brain stimulation on brain functional networks associated with high cognitive performance are provided. The data refer to a similar protocol as the one described for enhancement of brain resilience to perturbation, with an external perturbation in the form of an electromagnetic pulse delivered to the brain and brain data collected before and after the perturbation. Analysis of functional networks properties related to, but not limited to, information processing, integration and segregation of brain networks, synchronization (connectivity) within and between functional networks, are reported, with a significant increase of brain fitness after targeted personalized perturbation.

Analysis of brain efficiency and network dynamics included characteristic path length (the average distance between a node and all the other nodes of the system) that may represent how easy the information can move within the network; the nodal degree (total number of edges that are connected to a given node), global efficiency and local efficiency, the diffusion efficiency, the clustering coefficient (as the fraction of nodes being neighbors with the surrounding nodes, forming triangular triplets), and the small-worldness or the property of a system to have concomitant high clustering coefficient and low path length. Changes in the number of the modules and the corresponding modularity index expressing how much each structural connectivity matrix was arranged in sub-modules as computed using the Louvain algorithm were also examined.

FIG. 8 shows three different graph measures representing integration and segregation of information processing (e.g., Characteristic Path Length, Modularity, Global and Local Efficiency). The brain network appeared to be more connected shortly after brain perturbation and subsequently going back to baseline after stimulation. A decrease in Characteristic Path Length represents a facilitation in how information can move within the network after each perturbation. This result is also confirmed by modifications of the topology of the brain network, as captured by Nodal Degree, Clustering Coefficient, Modularity Score, and Number of Modules. Note: *p<0.05.

### Personalization of Neuromodulation Targets for Optimal Brain Control

In some embodiments, DARWIN may be used to identify optimal brain structures, areas and networks exerting a controlling effect on brain activity, and therefore may be important to induce changes in brain state. Identification of such structures and functional networks may allow for performing a selective perturbation aimed at increasing levels of brain control, thus inducing, for instance, a specific change in brain state or an overall decrease in brain controllability (e.g., resulting in enhanced shielding against perturbation). Analysis of network controllability was performed by representing the human brain as a network, based on both its structural and functional connectome. The resulting connectome matrices were used to define preferential connectivity pathways supporting information processing and hierarchical organization of brain activity.

DARWIN may be used to perform Brain Control Analysis (BCA). BCA allows to probe a system's ability to drive its output towards a desired outcome through the application of suitable input signals to selected nodes. The temporal evolution of the underlying neural system denotes the states of N nodes (neurons or brain regions) at a given time describes the nonlinear dynamics of each node/region, accounting for the impact of an external stimuli, e.g. in the form of noninvasive brain stimulation, applied to an input node(s). The goal of BCA is to identify M nodes (driver nodes) able to guide the system towards a desired final state. When applied to the brain via BCA, identifying driver nodes can inform the selection of which brain regions should be stimulated via NIBS to ensure that the brain can be influenced into a desired direction/state.

BCA was applied, within the DARWIN framework, to functional EEG data of healthy individuals for the identification of optimal network nodes able to induce a transition from a spontaneous mind wandering state to an enhanced cognitive control state. Analysis was performed by identifying personalized driver nodes for each individual, minimizing the control energy needed to reach the desired brain state. The study involved optimization of an intervention based on noninvasive brain stimulation, in which a single node/area/network is targeted at any given time or, alternatively, multiple nodes/areas/networks are targeted.

BCA was able to identify specific driver nodes for each individual, with a variability across individuals of ˜78% and only approximately ¼ of the sample receiving simulated stimulation on the same node/area/network. A comparison of brain controllability results in response to noninvasive brain stimulation performed over personalized vs standard (i.e., same for every individual) targets, showed an 81% increase in brain controllability when using personalized vs standard targets. The most represented brain targets for successful brain state transition from a mind wandering state to an enhanced cognitive control state were identified as the anterior cingulate cortex, the left dorsolateral prefrontal cortex, the inferior frontal gyrus, and the superior parietal lobe. Biophysical modeling performed in brain scans of the participants was used to identify the brain targets with the highest potential of being targeted effectively given their location and morphology in the brain. Results suggested the left dorsolateral prefrontal cortex and superior parietal lobe as the main targets to achieve brain state control in 79% of the participants while guaranteeing an induced electrical field (i.e., stimulation) of at least 0.25V/m, necessary for inducing brain activity.

Additional analyses were performed to investigate the possibility of transition to other brain states, including transition to a memory retrieval state relevant for patients with Alzheimer's disease, and transitions to a high creativity state and a mood-control state relevant for patients with depression. BCA was able to identify optimal brain targets for each condition, with successful state transition achieved in 88%, 68%, and 79% respectively.

In this example, it was demonstrated that BCA can be used to identify brain targets that when stimulated may induce a controlled change in brain state in humans. Data processed with PREPARE and analyzed via DARWIN offer the opportunity to identify personalized brain stimulation targets for state controls applicable to both cognitive enhancement and therapeutic applications.

### Connectome-based Analysis of Tumor Migration Pathways and Prediction of Clinical Outcomes

Due to limited therapeutic options, incidence in relatively young people, delayed diagnosis, and infiltration into the brain parenchyma, malignant brain tumors rank fourth among all cancers in terms of number of years of life lost, despite representing only 2% of all cancers. In particular, glioblastoma (GBM) is the most frequent and aggressive high-grade glioma (HGG, WHO glioma IV), with a mean survival of approximately 16-18 months from diagnosis.

The recent discovery of neuron-to-glioma synaptogenesis offers the possibility for novel strategies to interfere with this new pathophysiological behavior. Neuron-to-glioma communication is not unilateral, and even if glioma cells are not able to spike, they have been found to promote neuronal firing with the purpose of creating positive feedback with neurons for their further activation via multiple mechanisms, such as synaptogenic factor secretion, non-synaptic glutamate release, and by reducing the activity of inhibitory interneurons in the surrounding microenvironment. Animal experiments have further confirmed and extended these data by showing increased high-frequency (70-110 Hz) activity-an index of neuronal activation-in infiltrated tissue of patients with HGG. These changes expand to both meso- and macro-brain level, with resulting changes in the activity of brain functional networks.

The systems and methods described herein, using DMDT and DARWIN submodules, includes algorithms to localize and map brain tumors based on brain scans and electrophysiology data, characterize their behavior and relationship to clinical symptoms, predict their migration trajectories in the brain and may be used to inform on optimal targets for therapeutic neuromodulation interventions. When targets are identified via DARWIN, brain stimulation may be performed, e.g., according to STIMOLA.

In some embodiments, NiBS may be used to modulate the positive feedback mechanism between increased neuronal excitation triggered by gliomas and its impact on mitosis and migration. Controlling neuronal excitability in patients with HGG may inhibit tumor growth and proliferation, ultimately prolonging patient survival. In some embodiments, NiBS techniques such as TMS and tES may be used for their ability to induce LTD-like changes in synaptic excitability, relevant in the neuron-to-glioma context, where reduced probability of neuronal firing after a presynaptic event would reduce the activation of Ca2+AMPA-R in the post-synaptic glioma cell, leading to a limited inflow of Ca2+ signal mitosis-promoting and thus potentially limiting the neuronal contribution to glioma growth. In some embodiments, NiBS may be used to modulate/suppress synaptic signaling of neurons surrounding an HGG tumor, thereby, for example, slowing down its mitosis and migration rate. In some embodiments, NiBS may be applied to modulate tumor perfusion and blood-brain barrier permeability, thereby, for example, increasing the efficacy of chemotherapy. In some embodiments, inhibitory tDCS may be used to reduce the probability of neuronal spiking in the targeted cortical areas via LTD-like effects, thereby, for example, interrupting diffuse neuron-to-glioma communication.

In some embodiments, STIMOLA may be used to transiently increase the permeability of the blood-brain barrier to small and large molecules, thereby, for example, enhancing the delivery of drugs through the barrier.

In some embodiments, STIMOLA may be used to control tumor cells migration and spreading leveraging galvanotaxis principles. Cells can be oriented and guided in their migration when exposed to electric fields (galvanotaxis). In some embodiments, the cathodal field generated by tDCS in the region surrounding the brain tumor may be used to restrict migration of cancer cells localized in the cathodal field (e.g., in the immediate vicinity of the tumor borders), thereby, for example, interfering with migration and infiltration across the brain. In some embodiments, STIMOLA may be used to suppress neuronal tumor-promoting activity via inhibitory NiBS protocols including but not limited to cathodal tDCS, closed-loop anti-phase tACS, and/or continuous theta burst stimulation.

In some embodiments, BRAINPRINT, DARWIN, and STIMOLA may be used to identify optimal stimulation targets to suppress tumor activity based on location of the tumor and its brain connectivity profile with respect to known brain functional networks. The possibility of interacting with an entire network rather than with a single brain area could help in suppressing the neural activity regulating cancer growth and tumor spread. Inhibition, of an entire network could be more effective in slowing neuronal-related cancer growth, with tools derived from network control theory potentially representing a valuable approach to select the most relevant stimulation targets.

In some embodiments, BRAINPRINT and DARWIN may be used to identify optimal stimulation targets to suppress tumor activity. Information derived from the structural and functional connectome data of a patient may be used to map potential migration pathways of a solid tumor, based on white matter tracts connections and the tumor's profile of functional synchronization/connectivity. Tools may include, but are not limited to, network analysis, network control theory, graph theory and evolutionary biology. Analysis of longitudinal changes in brain network dynamics collected via neuroimaging and electrophysiology techniques, including changes in network topology, may be used to inform a predictive model identifying the most probable brain regions affected by tumor migration.

In some embodiments, BRAINPRINT and DARWIN may be used to identify patterns of connectivity between the tumor mass and the rest of the brain holding predictive power over clinical status and disease trajectory including, but not limited to, a patient's overall survival. Resulting patterns of connectivity may be used as targets for NIBS interventions aimed at shutting down connectivity, slowing down tumor aggressiveness and increase survival.

In some embodiments, BRAINPRINT, DARWIN, and STIMOLA may be used to identify brain networks associated with tumor symptomatology including, but not limited to, cognitive deficits. STIMOLA may be used to modulate network activity to slow down cognitive impairment or enhance recovery.

In some embodiments, NiBS may be used to restore the excitation/inhibition (E/I) balance of brain network(s). Cortical E/I ratio is typically altered in patients with gliomas, as well as in other neurological and psychiatric conditions, often associated with cognitive deficits and symptoms. In patients with gliomas, the E/I imbalance is involved with the emergence of epileptiform activity, and with subsequent neuronal death, paralleled by tumor progression.

In some embodiments, STIMOLA may be used to enhance the effect of drug therapies and chemical agents, including using magnetic or electrical stimulation to modulate the blood-brain-barrier (BBB) permeability and improve transfer and absorption of molecules, for instance chemotherapy agents. STIMOLA may be used to modulate local or distributed brain perfusion to enhance the effect of chemotherapy agents, by modulating neurovascular coupling in the tumor or the surrounding environment.

In some embodiments, DMDT and DARWIN may be used to extract indexes of a tumor's functional behavior via analysis of dynamic connectivity pattern within the tumor mass and in the surrounding brain tissue, including but not limited to white and grey matter. Dynamic connectivity metrics may include measures of similarity of tumor's activity with the activity of other brain regions, including those close to the tumor and those located in the rest of the brain. Similarity of spontaneous and evoked activity is an index of a tumor's invasiveness, and in some embodiments is calculated as the proportion of data recording displaying tumor's activity being synchronized with that of healthy brain structures over time. Parcellation of brain regions in networks and modules may also be performed, allowing to estimate metrics including but not limited to: (i) labelling a tumor's activity as belonging to a specific network or module over time, (ii) estimating the number of times the tumor's mass has switched its activity resulting in increased similarity with a different network or module, (iii) estimating the stationarity of tumor's activity over time, (iv) estimating the potential for the tumor mass to evolve its behavior over time as a function of its complexity features, thus providing an index of the tumor's aggressiveness. Additional metrics may be extracted from tumor's data including but not limited to those described herein as part of DMDT and DARWIN modules.

In some embodiments, data from DMDT is used to identify regions of potential tumor recurrence by analyzing patterns of functional connectivity within the infiltrated tissue around the tumor lesion (e.g., edema). An index of functional connectivity representing the strength of correlation between each voxel of an edema mask and the rest of the brain may be calculated using functional MRI BOLD data, the resulting quantitative map capturing voxels and clusters of voxels displaying positive or negative connectivity with the rest of the brain; an algorithm compares the pattern of connectivity of each voxel with that of voxels belonging to a solid tumor mask (comprising only the tumor mass) and a similarity score may be obtained. Voxels and clusters of voxels with high similarity may be labeled as potential locations for tumor recurrence due to their similar connectivity profile which reflects tumor infiltration.

In some embodiments, the same approach described in the previous embodiment is used with diffusion imaging data instead of functional MRI BOLD data. Maps of diffusion are created, resulting in voxel-wise representations of metrics including but not limited to fractional anisotropy, mean diffusivity, free water and number of streamlines. An algorithm may compare the pattern of diffusion of each voxel in the edema mask with that of voxels belonging to the solid tumor mask (comprising only the tumor mass) and a similarity score may be obtained. Voxels and clusters of voxels with high similarity may be labeled as potential locations for tumor recurrence due to their similar diffusion profile which reflects tumor infiltration.

In some embodiments, a videogame application is created to guide an individual's mental thinking and reasoning pattern, in order to selectively engage specific brain regions while avoiding activating others. The selection of regions may be based on DMDT and DARWIN data extracted from an individual with brain tumors including, but not limited to, glioma, glioblastoma, astrocytoma, oligodendroglioma, and melanoma brain metastasis, for their involvement with the tumor's network. The tumor network may be defined as a set of brain regions located in one or both hemispheres, connected with the tumor via structural connection (including white matter fiber tracts) or functional connection (including those measured via functional MRI imaging—functional connectivity). The videogame may be configured to induce a selective activation of regions not connected to the tumor, preventing the tumor from receiving additional stimulation/activation and induce tumor proliferation and migration. Brain tumor cells have been shown to migrate along white matter tracts, and establish connection with regions displaying high functional connectivity with the tumor; activation of the brain regions surrounding the tumor increases blood flow and metabolism in the area, also feeding tumor cells; avoiding direct tumor activation and activation of regions connected to the tumor is the goal of the videogame application, to prevent tumor growth and spread.

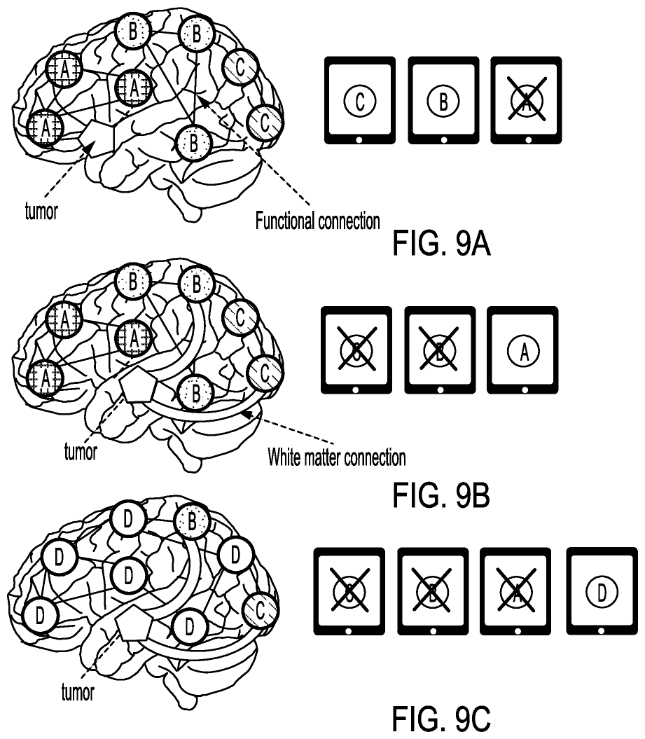

In some embodiments, the DMDT and DARWIN data of a patient with a brain tumor may be analyzed to create a map of regions to avoid and one or more regions whose activation will not affect the tumor (e.g., target regions). FIGS. 9A-9C schematically describe a cognitive engagement application in accordance with some embodiments of the present disclosure. Cognitive functions corresponding to the target regions may be identified (e.g., via IMPROVE) and ad-hoc cognitive tasks may be created to selectively activate these functions. DMDT and DARWIN may also identify brain regions (and corresponding cognitive activities) to avoid during the day, either based on proximity to the tumor (FIG. 9A) or because of their connections with the tumor (FIG. 9B). The tasks supported by the target region(s) may then be embedded into a game to increase playability and ensure adherence to the treatment. The individual with a brain tumor may be asked to play the game for a predefined duration per day, in specific moments, to maximize activation of the target region(s) and minimize tumor activation as much as possible throughout the day.

In some embodiments, DMDT and DARWIN are used to map a connectivity profile of a brain tumor, and identify regions connected to the tumor mass as shown in FIGS. 9A-9C. Depending on tumor's location and connectivity profile, an application may suggest one or more brain regions to be avoided because of their functional connectivity profile (regions “A” in FIG. 9A), structural connectivity profile (regions “B”, “C” in FIG. 9B), and may identify clusters of regions to be engaged to maximize brain activity unrelated to the tumor's activity (regions “D” in FIG. 9C).

### Brain Tumor Monitoring via Spontaneous and Evoked Brain Activity Analysis

Brain tumors including, but not limited to, gliomas, glioblastomas, astrocytoma, and oligodendrogliomas, induce significant changes to brain structure and function, due to mechanical pressure, metabolic influence on surrounding healthy brain tissue, and/or alteration of brain synchronization patterns important for cognitive functions. Brain tumors are among the most aggressive tumors, and therapeutic options are limited. Moreover, diagnostic options tend to be even more limited, with physicians being only able to diagnose brain tumors when patients request their consult after symptoms onset. Importantly, even after successful treatment of brain tumors via surgery, chemo-therapy and radiation therapy, brain tumors tend to reappear (tumor recurrence), with the rate of survival being dramatically smaller after the first and second tumor recurrence. Being able to detect new brain tumors and post-treatment recurrence may be important to successful treatment, longer survival and better quality of life; however, solutions for detection and monitoring of brain tumor's activity tend to be limited to expensive, hospital-based exams including but not limited to Magnetic Resonance Imaging (MRI) and Positron Emission Tomography (PET), which are requested only after symptoms onset or at fixed time points after surgery. Some embodiments of the present disclosure relate to a platform for remote, self-administered, brain activity monitoring that is able to capture changes in brain activity over time and identify patterns of abnormal brain activity indexing tumor activity.

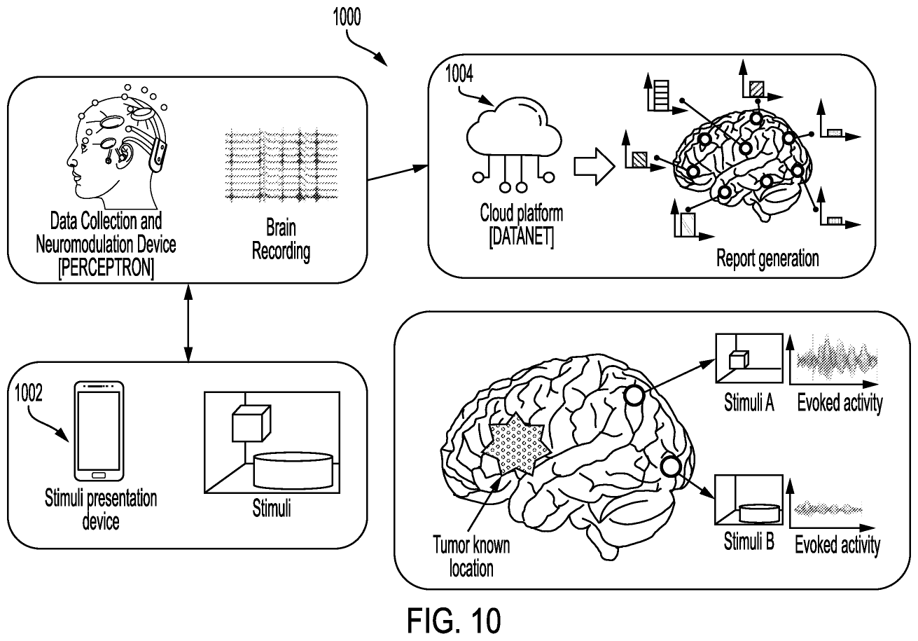

A device configured to measure brain activity non-invasively (e.g., [PERCEPTRON]) and a digital platform configured to deliver cognitive tasks activating specific brain regions may be included in a system for monitoring a brain tumor in accordance with some embodiments. An example of such a system is shown schematically in FIG. 10. An individual response to each cognitive task generates a map of spontaneous and evoked brain activity, which is recorded over time and inform the individual DMDT. Some embodiments relate to a specific approach to combine information from each brain response, both cross-sectionally and longitudinally, to generate a score of brain activity which is then used to assign a probability of tumor presence, recurrence and migration.

In some embodiments, brain tumor activity is quantified via cognitive tasks performed on portable device including, but not limited to, a personal computer, tablet and mobile phone. The cognitive tasks may induce specific patterns of brain activity that are recorded via a portable device to capture brain activity (e.g., [PERCEPTRON]), generating specific profiles of responses depending on the type of stimuli presented to the individual performing the tasks. The tasks may include, but are not limited to, simple stimuli presentations including, but not limited to, visual and auditory stimuli presented in a rhythmic fashion on the screen and/or via speakerphones or headphones. The tasks may include, but are not limited to, specific classes of visual and auditory stimuli including, but not limited to, objects, persons, animals, and their subclasses (including but not limited to living and non-living objects). Specific stimuli may be chosen to evoke specific patterns of brain activity detected using methods including, but not limited to, scalp electroencephalography (EEG) (e.g., [PERCEPTRON]), in the form of Event Related Potentials (ERPs). The stimuli may be selected to evoke activity in specific brain regions, including all brain lobes and both brain hemispheres.

In some embodiments, the EEG signal is analyzed by analyzing specific brain waves generated in specific time windows before, during and after the presentation of the stimuli. Brain waves characteristics including, but not limited to, amplitude, latency, frequency and coherence across EEG channels may be quantified and used to create a user-specific fingerprint of response (e.g., [DMDT]).

In some embodiments, the location of specific responses, their characteristics, and/or their change over time, may be used to generate a map of brain activity reflecting the influence of brain tumor's presence on brain activity. Longitudinal monitoring of the change in brain activity may be used to determine patterns of deviation in brain activity associated with tumor recurrence, progression or migration. The information may be used for early detection of tumor recurrence and progression, and may trigger more in-depth investigation by a tumor specialist including a neurooncologist, neurologist, neurosurgeon, or neuroradiologist. The information about the changes may be used for mapping of tumor migration, identifying brain regions where the tumor is migrating based on altered patterns of brain activity.

In some embodiments, the metrics extracted from evoked and spontaneous brain activity may include, but are not limited to, amplitude of evoked spikes and peaks in brain signal; latency of spikes and peaks in brain signal; frequency of oscillatory activity; level of synchrony between activity recorded over multiple brain locations (functional connectivity); influence of activity evoked at one brain region over activity evoked from another brain region (causal connectivity); algorithmic complexity of the signal; and/or delay between the onset of a stimuli and the time brain activity return to a pre-stimuli activity level (brain responsiveness).

In some embodiments, the metrics extracted from evoked and spontaneous brain activity may include metrics calculated via the DARWIN module (including, at least in part, brain plasticity, resilience, efficiency, and/or evolvability), and other perturbation-based metrics described herein in response to TMS and TCS.

In some embodiments, the assessment may be performed over different timescales, ranging from once a day to once a year, may be performed based on an established schedule or may be performed in response to the onset of symptoms possibly related to tumor onset, recurrence, migration, including but not limited to cognitive deficits and health changes.

In some embodiments, data collected locally during task execution and brain recording (e.g., using PERCEPTRON) may be uploaded to a cloud-based analysis platform (e.g., DATANET), where spontaneous and evoked brain activity may be processed and analyzed. In some embodiments, the results of the assessment may be summarized in a report, reporting the level of activity and likelihood of tumor onset, recurrence and migration, over multiple brain regions and networks. In some embodiments, the report may be used to determine targets for therapeutic or preventative interventions aimed at interrupting tumor migration, slow down tumor growth or counteract cognitive symptoms.

FIG. 10 shows a brain tumor monitoring platform 1000 in accordance with some embodiments of the present disclosure. Brain activity may be recorded before, during and after stimuli are presented via a portable stimuli presentation device 1002. Stimuli may be presented in order to evoke specific patterns of brain activity in different brain regions, networks and systems, evoking different patterns of activity (example, Stimuli A evokes strong activity in a specific brain region, whereas Stimuli B evokes low brain activity in a different region). Activity may be monitored over time, with assessments performed over the course of a day, weeks, months and years. Stimuli-related data and brain recordings may be processed in a cloud-based platform 1004 (e.g., DATANET) and a report of tumor-related brain activity may be generated.

### DMDT and DARWIN for Consciousness Sampling and Classification

Assessment of brain, cognitive and psychological state, including evaluation of performance-related brain state (flow state) and pathological mental states is typically performed based on outdated standards and tools, including questionnaires and self-report measures. The implementation of wearables has opened to novel applications for state detection, but direct access to brain data is needed for prediction of brain state change and identification of state-markers. Moreover, methods and algorithms for state-analysis can also be used to evaluate the impact of interventions used to modify brain states. In this example, methods from DMDT, DARWIN and SCREEN are used for consciousness sampling and classification (CSC), based on EEG data and other non-invasive electrophysiology recordings including, but not limited to, galvanic skin response and cardiac output, leading to the creation of a consciousness assessment tool.

### Methods

Electroencephalography (EEG) and galvanic skin response (GSR) data from a sample of healthy participants was collected during multiple brain states induced via external instructions provided before the data collection. Participants were familiarized with the nature of each state and on how to induce them. Snapshots of brain and biosensing data were labeled according to each brain state, with brain states including at least in part states related to thinking about the future, thinking about the past, recalling a traumatic memory, recalling a pleasant memory, thinking about the present experience, focusing on individual body sensations, performing a memory exercise, performing a language exercise, thinking of a preferred physical or mental activity, thinking about family and friends, thinking on a previously assigned problem, thinking about art in the forms of paintings or sculptures, thinking about themselves, or solving a creativity puzzle.

### Dynamic Decomposition

Analysis of data was performed following procedures and methods described as part of the PREPARE module. A fingerprint of dynamic brain states was created by sampling segments of data corresponding to each specific state. A search for the spatio-temporal fingerprint of each state was then performed within the resting-state, unprompted, mind wandering data recorded at the beginning of the session. A machine learning algorithm was trained to identify each state within the spontaneous EEG and biosensing data, resulting in a quantification of time spent in each state during spontaneous mind wandering. The same fingerprinting/labeling procedure was applied to data collected after a VR-based trauma reconciliation intervention aimed at alleviating symptoms related to past traumas in the context of a condition of post-traumatic stress disorder. Participants were exposed to multiple sessions of trauma reconciliation, EEG and biosensing data were collected before and after the series of sessions.

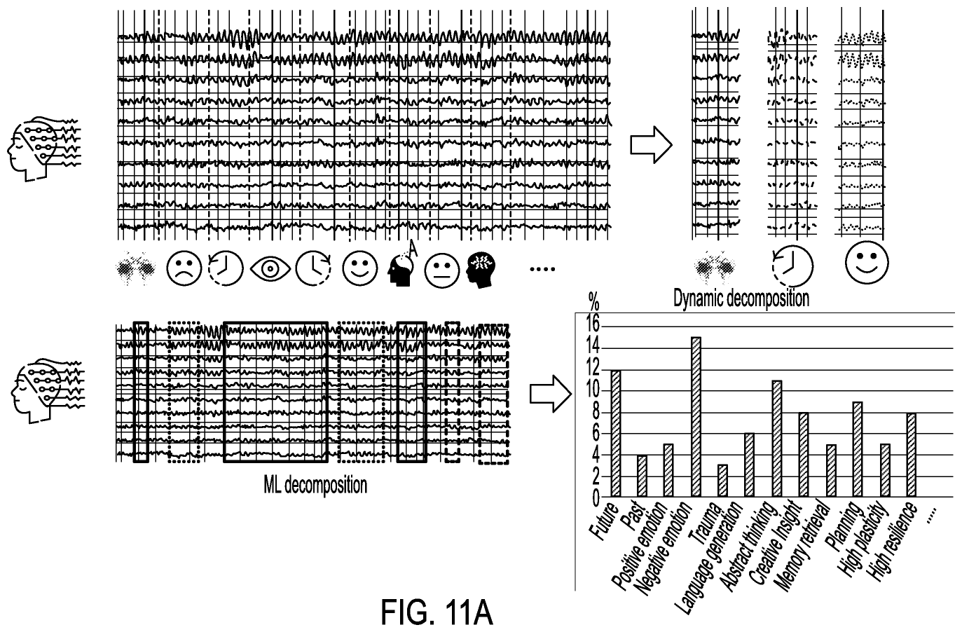

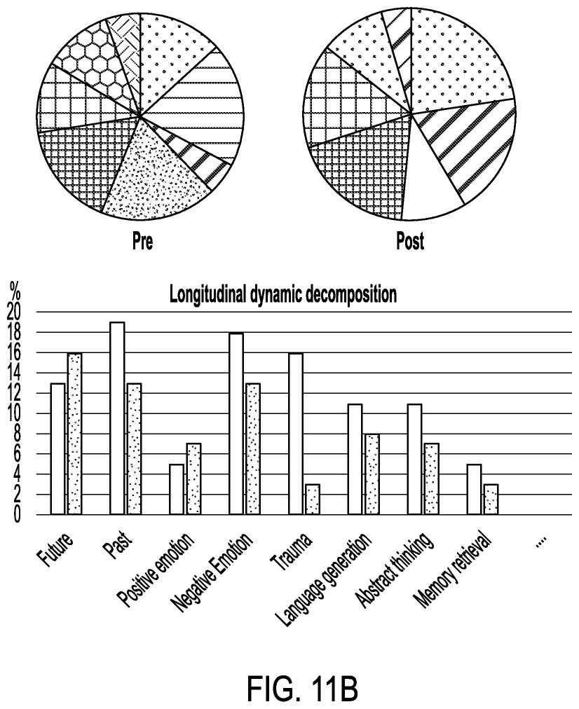

FIGS. 11A-11B show an example of dynamic decomposition for psycho-cognitive assessment in accordance with some embodiments. FIG. 11A shows data from DMDT that includes brain data collected during various externally induced brain states, as well as data collected during spontaneous mentation. Previously-identified brain states were mapped onto spontaneous mentation segments, resulting in a quantification of states and relative representation during un-prompted mind wandering. States were identified via data processing via PREPARE, involving extraction of data features related to multiple dimensions, including at least in part time and frequency, network metrics and complexity. FIG. 11B shows that exposure to an intervention aimed at suppressing trauma-related memories and negative thoughts, resulted in structural remodeling of spontaneous mentation with a change in target brain states.

### Results

As shown in FIG. 11A, characterization of brain states and their labeling via machine learning was achieved with 83% accuracy, with pre-labeled states addressing for approximately 78% of available spontaneous EEG data across participants. The decomposer failed to classify on average 22% of the data, with a range of 11%-39%. Hierarchical estimates related to each state also correlated with self-report measures of each state and its estimated % of presence during spontaneous mentation.

As shown in FIG. 11B, overall, subjects exposed to mind-stream training showed an increased sense of control and awareness on their stream of consciousness, regardless of the type of training. Participants exposed to the VR intervention displayed a change in the structure of spontaneous mentation, with a reduction of trauma-related brain states and states associated with negative thoughts and negative emotions. A change in the ratio between brain states indexing thought about the future and those about the past was also observed.

The results of this example demonstrate that dynamic brain state decomposition via consciousness sampling and classification (CSC) is an effective method for passive, data driven assessment of internal mentation that may be used, at least in part, to identify severity of mental illness beyond conventional verbal/interview/self-report methods. The method may be used, at least in part, to track longitudinal changes in brain states compositions and related psychological symptoms and dimensions.

In some embodiments, CSC is performed in combination with perturbation-based biomarkers to evaluate the brain state of patients with disorders of consciousness including, but not limited to, vegetative state, coma, emerging vegetative state, locked-in syndrome. Perturbation may be applied to the body and/or brain via presentation of external stimulation including, but not limited to, transcranial electrical stimulation, transcranial magnetic stimulation, electrical peripheral stimulation, sensory stimulation including auditory, visual and tactile stimulation (e.g., vibration, pulsatory mechanical pressure). A device for recording of brain data (e.g., [PERCEPTRON]) may be used to sample brain activity in response to external perturbation; brain data may be analyzed via PREPARE and then combined with time-locked information related to external stimulation, resulting in patterns of brain activity related to stimuli presentation. The system may generate a report of brain activity for different types of stimulation, that may be repeated over time to create a longitudinal profile used to identify shifts in brain activity representing a shift in consciousness levels. In an example application, a patient in vegetative state may be presented with auditory and vibrotactile stimulation on different body parts, including upper and lower limbs, and brain activity may be recorded via PERCEPTRON; changes in brain activity immediately following each stimuli may be recorded and stored in the patient's DMDT for off-line data analysis. The patient's brain activity in response to stimulation may be correlated with clinical information to determine an individual brain consciousness index (BCI) that is then monitored over time via repeated assessments. The method may be used to sample brain activity over different consciousness levels to determine level-to-level thresholds used to track consciousness level over time and identify trajectories of brain state change from the current consciousness level (e.g., vegetative state) to a different state (e.g., emerging vegetative state).

In some embodiments, the BCI includes information on brain response to stimuli related to specific brain regions, systems and networks, which may be used to monitor longitudinal changes in local brain activity in relation to changes in consciousness level. In some embodiments, this information may be used to identify targets for neuromodulation intervention via STIMOLA, to accelerate state-to-state transition via targeted brain stimulation interventions including, but not limited to, electrical stimulation delivered via PERCEPTRON.

In some embodiments, the VR-based intervention for trauma-reduction may be used, at least in part, to reduce symptomatology and obtain quantitative measures of psychological distress via features extracted as part of DARWIN. In some embodiments, dynamic brain state decomposition may be used to sample spontaneous mind wandering in healthy participants of various age to detect fingerprints of state-space modulated by aging, thus resulting in an index of brain and cognitive age related to brain aging and brain health. In some embodiments, dynamic brain state decomposition may be used to sample spontaneous mind wandering in patients with neurological conditions, in particular patients with dementia where the brain and cognitive age index described above may be used to predict the onset and progression of dementia including, but not limited to, Alzheimer's disease and Mild Cognitive Impairment. In some embodiments, dynamic brain state decomposition may be used to sample spontaneous mind wandering in patients with mood disorders including major depression, and in patients with anxiety-related conditions, as a biomarker of disease onset, progression and response to therapeutic interventions.

### NEUROMORPHIC ARTIFICIAL INTELLIGENCE [NEURO-AI]

The quest for the creation of a brain-inspired A.I. has been challenged by both the lack of global models of human intelligence, and the need for large amount of brain, cognitive and behavioral data to inform A.I. solutions. When brain data are available, cognitive and/or behavioral correlates tend to be lacking; vice-versa, when good cognitive and behavioral models of human problem solving are available, corresponding patterns of brain activity tend not to be available mostly due to high costs and lack of opportunities to collect such data in an organized fashion. As a result, true neuromorphic A.I. does not exist, limiting A. I.'s ability to show a significant impact on real-life tasks and challenges. One of the issues is represented by the search for a 1:1 replica of the human brain as the pinnacle of neuromorphic A.I., which has limited the evolution of brain informed A.I. over the past two decades. In order to, for instance, simulate high-order brain functions and network level dynamics, predict behavior and cognition, understand pathology and derive therapeutic treatments, a full carbon-copy of brain microcircuitry at the micro-scale (cellular) level may not be required. Rather, an optimal balance between the need for replication of brain features and the implementation of the actual organizational principles guiding such complexity may be identified and used to evolve current A.I. modules.

In this regard, the systems and methods described herein allow to (i) create a structured platform for acquisition and harmonization of meso-and macro-scale brain data, as well as corresponding cognitive and behavioral data (e.g., DMDT, PERCEPTRON), coupled with novel neuroscientific models of human intelligence elaborated by following the organizational principles derived via analysis of DMDT data (including at least in part the brain efficiency and plasticity indexes in DARWIN). In some embodiments, this allows to create a neuromorphic A.I. agent based on principles extracted from the DMDT of a single individual or a group of individuals, including modeling of oscillatory activity and micro-to-macroscale connectivity modeling. In some embodiments, this allows to create purpose-specific A.I. agents specialized on a specific task (e.g., idea generation, emotional support, negotiation), or general-purpose A.I. mimicking human reasoning abilities including logical and abstract reasoning. In some embodiments, this also allows to conceptualize cognitive architectures used to guide the creation of novel assessment tools as part of SCREEN.

### General-Purpose A.I.

Human cognition does not result from the brute, unorganized assembly of independent cognitive modules (e.g., memory module, attention module, abstract thinking module), but rather from the fine coordination of multiple cognitive streams flowing into a balanced architecture. Following this reasoning, a superior global-purpose A.I. able to tackle any problem may not be assembled by using, for instance, the best DMDT template for high memory performance, coupled together with the best DMDT of high attention capacity, and so on. Rather, an optimal balance may be found when organizational principles are followed.

In some embodiments, specific aspects of a DMDT and its DARWIN models may be used to create modules of a general-purpose A.I. (referred to herein as “NEURO-AI”), where the general structure and algorithmic wiring are defined based on data collected in individuals with high convergent and divergent thinking, constituting the optimal compromise between knowledge-based and intuition-based learning and problem solving. By using brain imaging and cognitive-behavioral data of healthy individuals, a model has been created that identifies a set of backbone brain regions supporting human convergent and divergent thinking, together with their preferential interaction pathways and dynamics. This model represents the wireframe for the generation of a novel class of A.I. where both elements of knowledge-based intelligence (convergent thinking) and creativity as well as intuition (divergent thinking) are represented and optimally tuned for the solution of different classes of problems. Additional details are provided in the section below entitled “Multi-layer Convergent-Divergent thinking (CDt) model of human cognition.”

In some embodiments, additional information may be implemented in NEURO-AI, including data on physiological and anatomical brain properties. For instance, information on local brain metabolic activity may be used to allocate energy to A.I. processing units following the DMDT of individuals with high CDt, whereas the organization of local and distributed information processing of a high CDt brain can be mimicked for resource allocation. Moreover, properties captured by DARWIN (including at least in part brain plasticity, resilience, efficiency, and/or evolvability) can also be used to define network topology of NEURO-AI, improving on the CDt model. For instance, characteristics related to brain resilience have been linked to higher cognitive performance in humans, suggesting how evolutionary dynamics might have shaped the human brain towards a high-resilience system where multiple hub brain regions are present thus decreasing the risk.

### Task-Specific A.I.

Following a similar principle as data-inspired model creation, but focusing on specific tasks and cognitive abilities, specific modules of NEURO-AI may be created by aggregating DMDTs and DARWIN data capturing specific cognitive functions of interest. In some embodiments, data of individuals with high inductive reasoning and executive functions is used to generate an AI solution for acute problem solving; data from individuals with high semantic access and high deductive reasoning is used to create an AI solution for medical diagnostics; data of individuals with high brain plasticity and learning abilities is used to generate an AI solution for adaptive learning in gaming applications; data from individuals with high insight abilities, semantic abilities and conceptual knowledge is used to create an AI solution for scientific inference, knowledge search and hypothesis generation (e.g., a virtual scientist).

### EXAMPLES

Some embodiments described herein are further illustrated by the following examples and detailed protocols. However, the examples are merely intended to illustrate embodiments and are not to be construed to limit the scope of the technology described herein.

### Neural Substrate of High Abstract Reasoning and Fluid Intelligence and Relevance for Enhancement

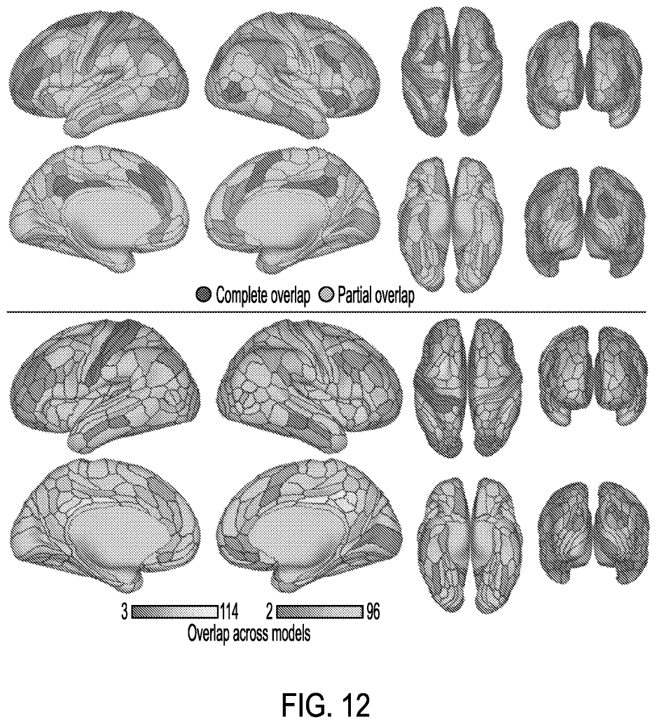

Analyzing brain spontaneous patterns of metabolic activity may be informative about—and even predict—individual evoked activity during sensorimotor and cognitive tasks. Such intrinsic activity is thought to reflect not only the past experiences of the brain as a system, but it may also form the functional foundation from which the brain will generate future goal-oriented behavior. Even though investigations about the functional connectivity (FC) correlates of fluid intelligence have been proposed, a clear overview of the role played by regions belonging to specific resting-state networks is not available. Below are the results of an analysis elucidating the most relevant brain regions supporting intelligence in humans, as well as their corresponding functional brain networks. Moreover, functional networks were classified into three main components supporting intelligence in humans, resulting in a list of targets for neuromodulation and enhancement of intelligence. Functional and structural MRI data were analyzed looking at local and inter-regional synchronization across a sample of healthy subjects, identifying regions and networks involved in any cognitive function relevant for intelligence (in red, FIG. 12), and those involved in some of the cognitive functions associated with intelligence.

FIG. 12 shows brain regions identified as supporting abstract reasoning and problem solving in humans using the systems and methods described herein. The top panel of FIG. 12 shows the most relevant regions (in red) supporting human fluid intelligence, across different cognitive domains and neuroimaging modalities. Regions in which activity supports some of the cognitive functions involved in fluid intelligence tasks are shown (in green). The bottom panel of FIG. 12 shows regions color-coded by their importance.

A hierarchy of regions and brain networks was identified as well, suggesting a wiring diagram for NEURO-AI and for task-specific A.I. related to convergent problem solving and hypothesis testing. Moreover, results also offered a framework for intelligence enhancement in humans, with specific brain targets and patterns of inter-regional synchronization to be optimized using DARWIN and neuromodulatory interventions from STIMOLA.

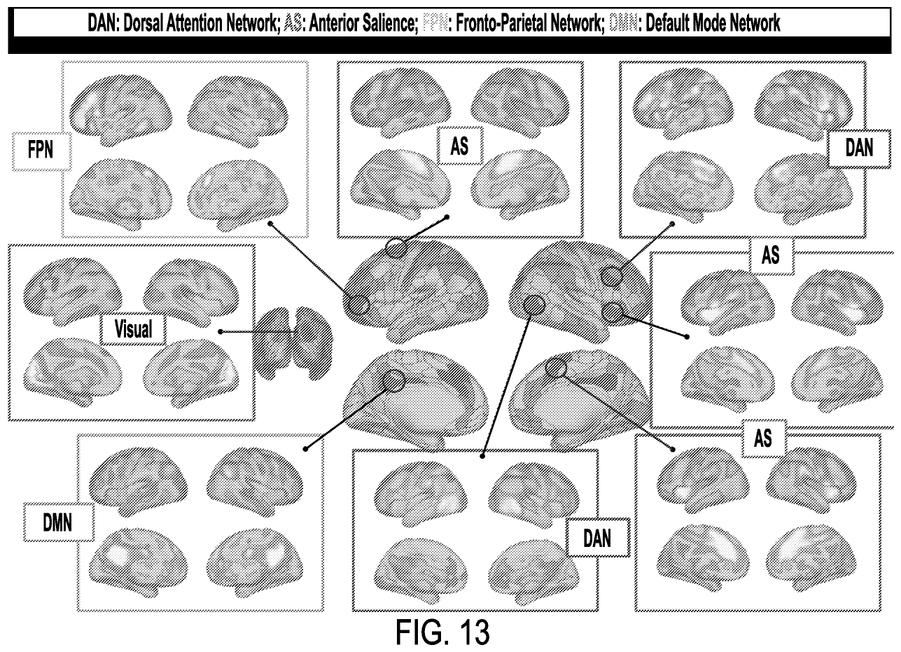

FIG. 13 shows brain networks identified as supporting abstract reasoning and problem solving in humans. The association between regions supporting intelligence and functional brain networks is shown.

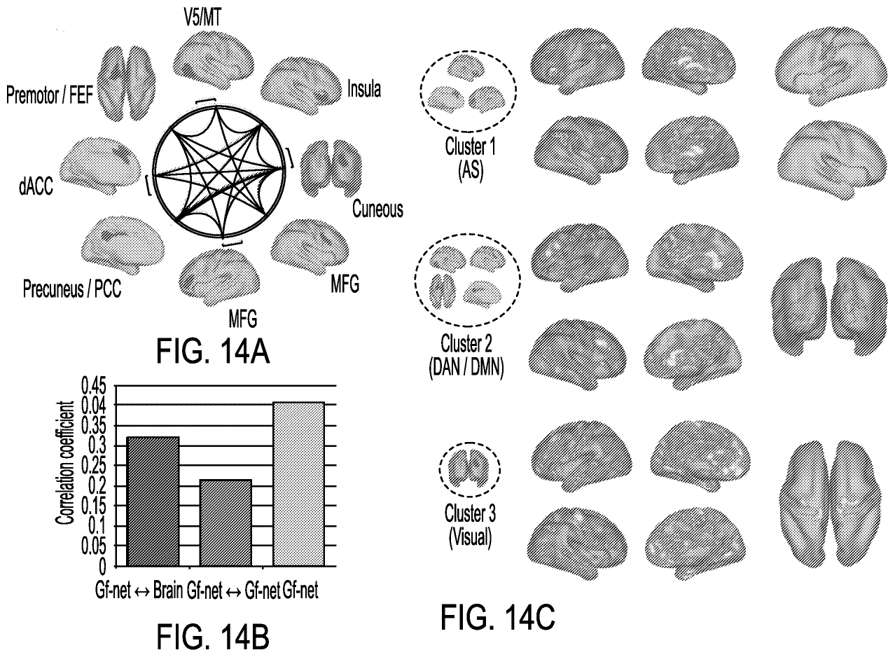

FIGS. 14A-14C show main clusters of brain networks supporting intelligence in humans. Regions and networks associated with intelligence were grouped in three main components based on their spontaneous activity and inter-network dynamics. The three components represent (i) areas related to the Anterior Salience (AS) network, involved at least in part in proprioception, perception of one owns' body and sensations, attention to internal signaling; (ii) areas related to the Dorsal Attention Network (DAN) and Default Mode Network (DMN), relevant for balancing attention towards external stimuli and internal dialogue; (iii) regions of the Visual Network, as a critical function of the human brain. The patterns of functional connectivity for each component are shown on the extreme right of FIG. 14C, showing how each component explains intelligence variability across subjects via its positive or negative synchronization with sensory regions.

### A Multi-Layer Convergent-Divergent Thinking (CDt) Model of Human Cognition

Human cognition includes a wide spectrum of abilities allowing for different problem-solving strategies depending on the task at hand. Convergent thinking, including so-called fluid intelligence (gf), characterize individuals able to find the only correct solution to a problem by means of logical and deductive reasoning. Divergent thinking, more associated to creativity and intuition (so-called insight), instead define individuals'able to produce a variety of possible solutions to an open-ended problem, a quality associated to artistic talent and geniality. Whether, regardless of their conceptual antagonism, the two cognitive domains share a common neuroanatomical substrate in the human brain is not known.

By analyzing the functional activation patterns related to convergent and divergent thinking, DMDT and DARWIN data were used to identify a subset of brain regions in the left inferior frontal gyrus, left frontal eye fields and bilateral anterior cingulate cortex, composing a domain-unspecific cognitive network correlated with both convergent and divergent thinking tasks. These overlapping regions also carry predictive value on a latent convergent/divergent thinking cognitive factor. The strength of the negative correlation between these brain regions/structures and activity of the default mode network was also identified as the best predictor of high convergent/divergent thinking in humans.

The results of this example promote the idea of a high-level backbone structure for abstract thinking in humans called the “Multilayer CDt model of human cognition”, subserving human cognition as a whole and therefore representing a crucial building block of a novel class of neuromorphic A.I. Such A.I. offers a platform where abilities and functions related to creativity, fluid intelligence and insight can be tuned to obtain heuristics best fitting the problem at hand.

Moreover, the CDt model also allows to identify optimal brain targets for cognitive enhancement to be modulated via neuromodulatory interventions including, but not limited to, noninvasive brain stimulation techniques.

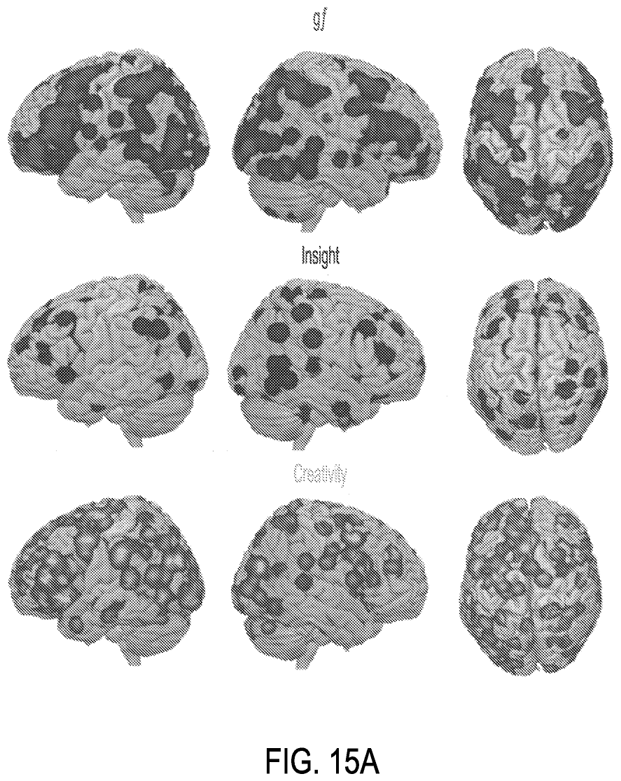

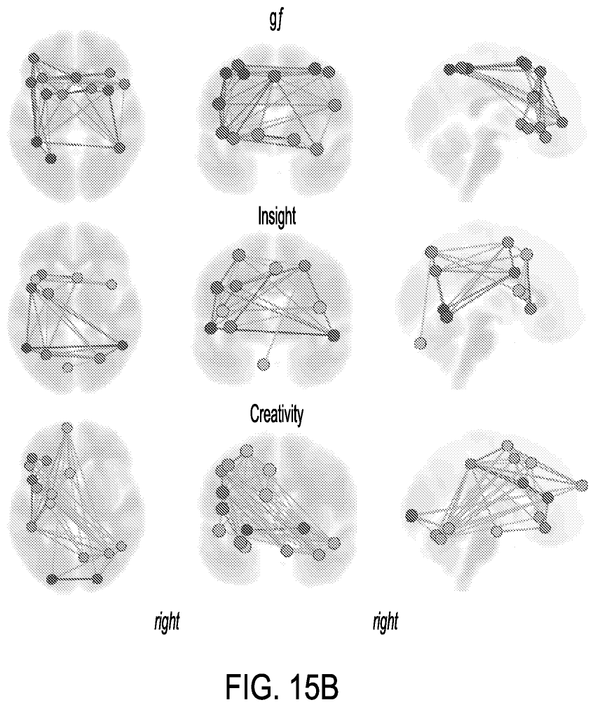

FIGS. 15-19 relate to CDt definition and results in accordance with some embodiments. Results were obtained by creating weighted maps reporting localization of the most relevant brain regions for gf, creativity and insight. The maps were then statistically compared, deriving areas of overlap between gf, creativity and insight as well as domain-specific regions, producing a quantitative answer to the matter of intelligence, creativity and insight being more overlapped than separated at the neurobiological level. Results show brain activations in both hemispheres for both convergent and divergent thinking, with no unique lateralization of the three functions examined. Functional brain data was analyzed by looking at the interaction between gf, creativity and insight nodes as well as other brain regions belonging to functional brain networks, using a clustering approach to identify similarities in their functional connectivity patterns. Finally, connectivity patterns of previously identified overlapping regions were correlated with behavioral gf, creativity and insight scores, as well as other lower-level cognitive functions. DARWIN was used to identify the best predictor of a global “abstract reasoning” latent factor in humans, with applications including, but not limited to, cognitive enhancement and accelerated learning.

FIGS. 15A-15B show activation foci and brain connectivity patterns. FIG. 15A shows activation foci for each domain across the two hemispheres. FIG. 15B shows significant brain regions composing each map following a permutation-based test (10000 permutations; p<0.05 FDR; cluster-based correction p<0.001), displaying the strength of their positive (yellow-red) and negative (cyan-blue) connectivity.

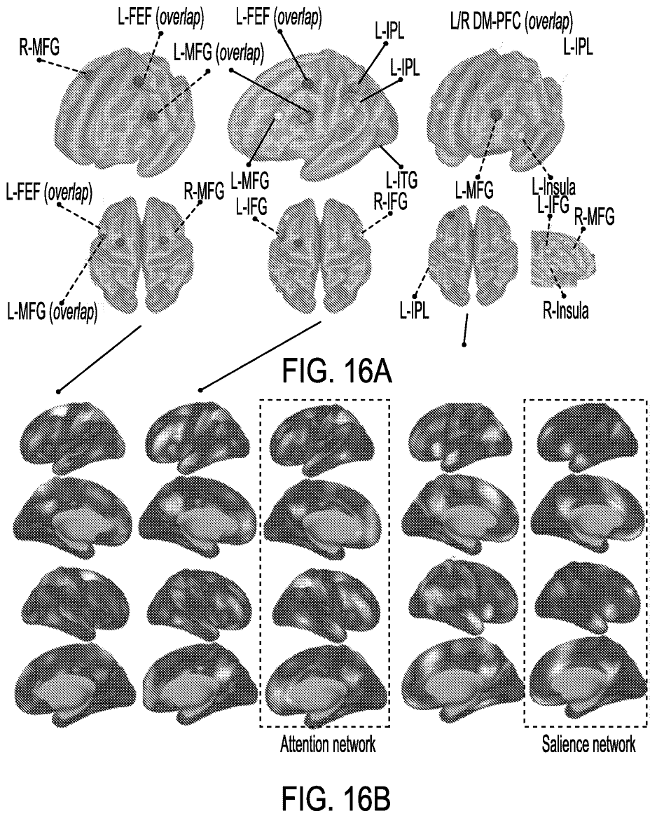

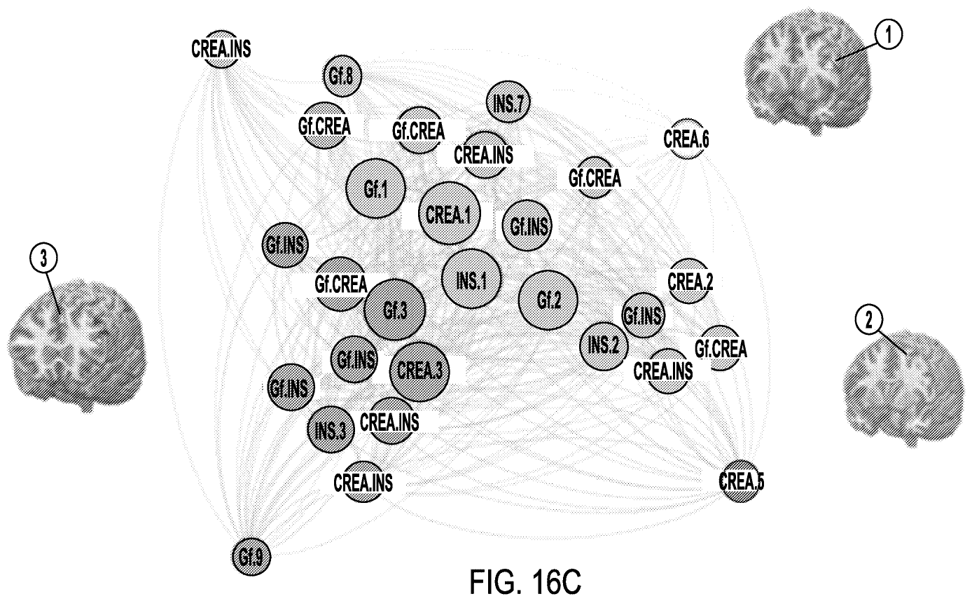

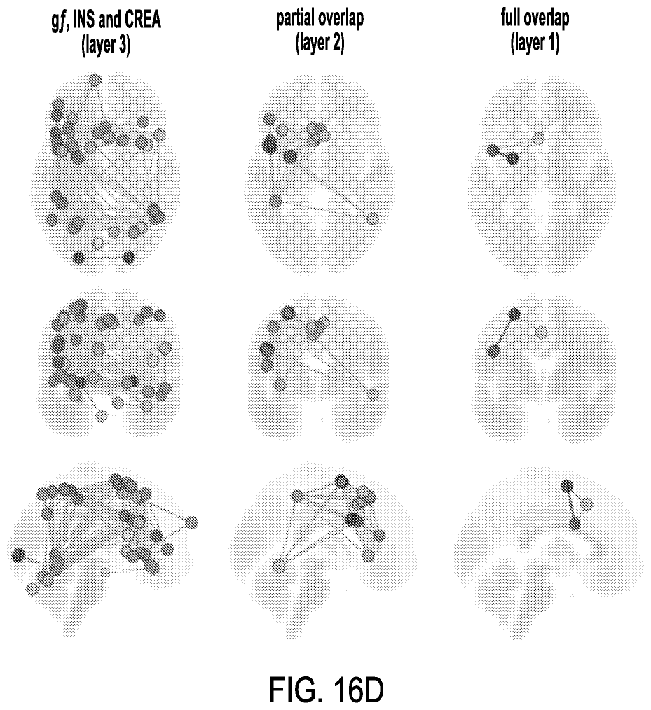

FIGS. 16A-16D show functional connectivity of overlapping nodes. Functional connectivity analysis including the three conjunction regions and nodes composing 14 brain functional networks (n=90) revealed a pattern of preferential connectivity at rest between two of the overlapping regions, i.e., left SFG and MFG. In FIG. 16A, black spheres represent overlapping nodes used as seed regions, red lines indicate the only significant connections out of all possible ones. FIG. 16B shows a seed-based connectivity profile of each overlapping nodes, resembling the attention and salience networks as highlighted by the dotted lines. FIG. 16C shows a modularity analysis highlighting a separation between the three fully overlapping regions and the rest of the ALE fMRI nodes. FIG. 16D shows a “Multilayer CDt model of human cognition.” The graphs in FIG. 16D show the connectivity between each node of (from left to right) the gf, creativity and insight maps (Layer 3), nodes being part of at least two maps (Layer 2), and those consistently reported in the three maps (Layer 1), which represents the “Multilayer CDt model of human cognition” in accordance with some embodiments.

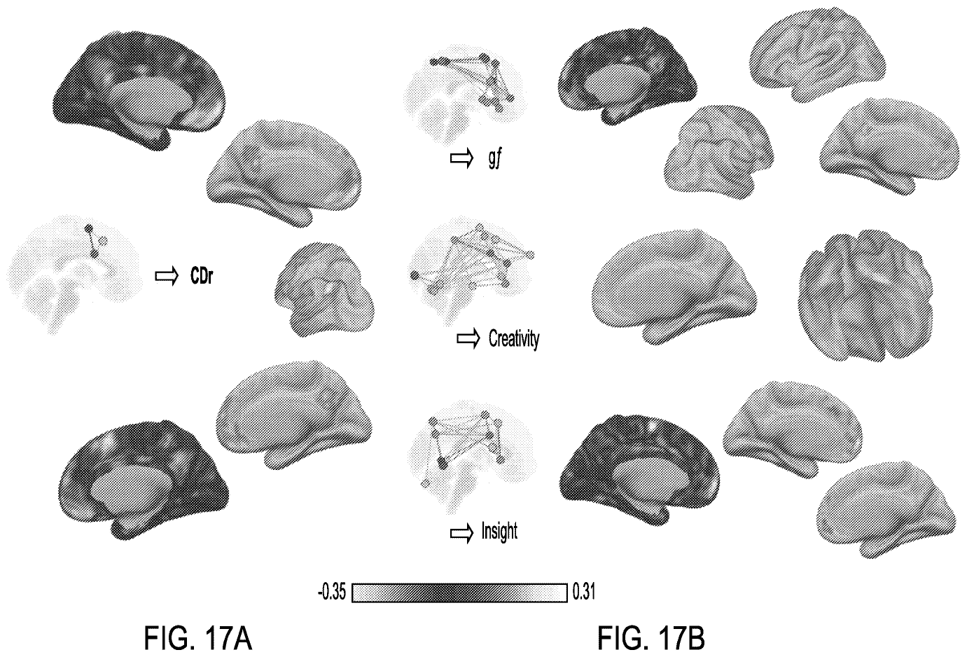

FIGS. 17A-17B show DARWIN analysis, connectivity and cognition. The negative correlation between Layer #1 regions and the default mode network correlates with individual CD-r scores, as shown in FIG. 17A. FIG. 17B shows a similar analysis using domain-specific meta-analytic maps and corresponding cognitive scores identifying the same negative correlation as best predictor of gf and insight scores, with a less similar pattern for creativity (p.<0.05 FDR; cluster-based correction p.<0.001).

### Brain-To-Command Engine

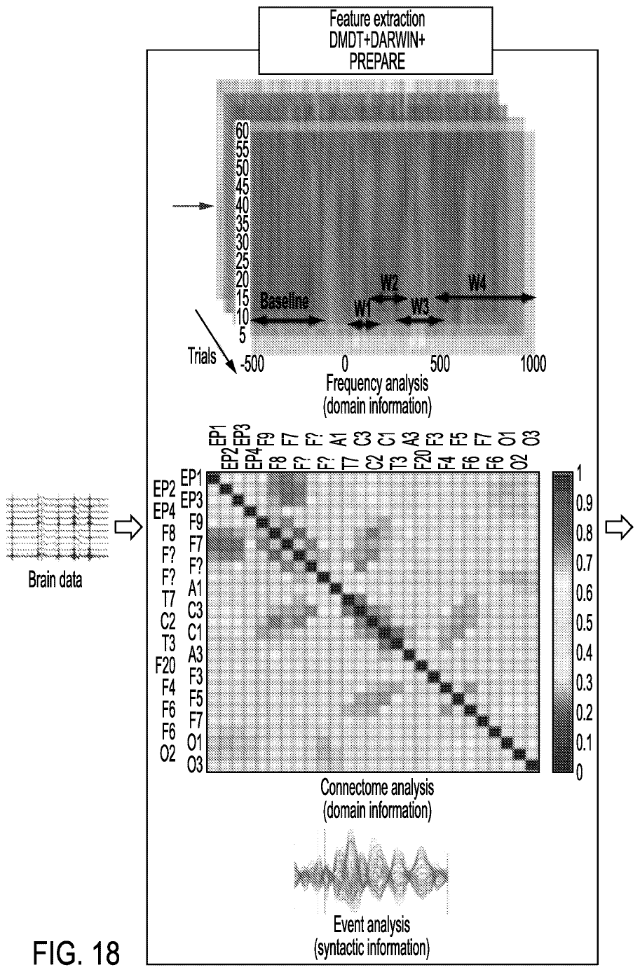

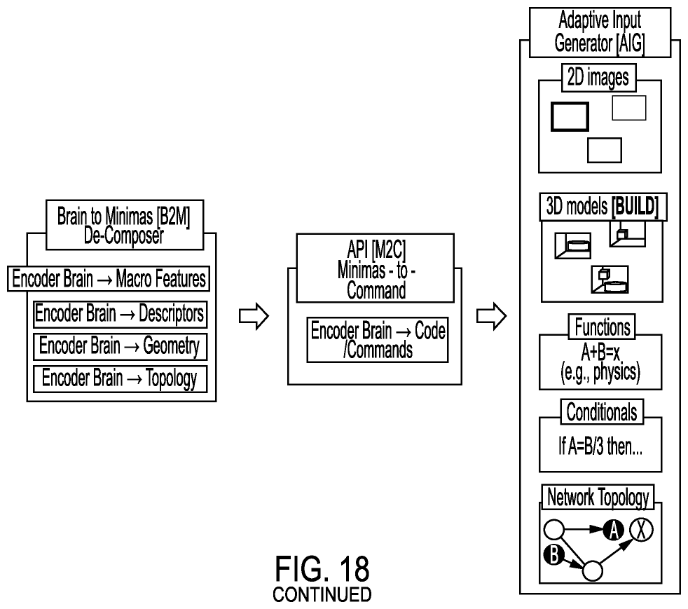

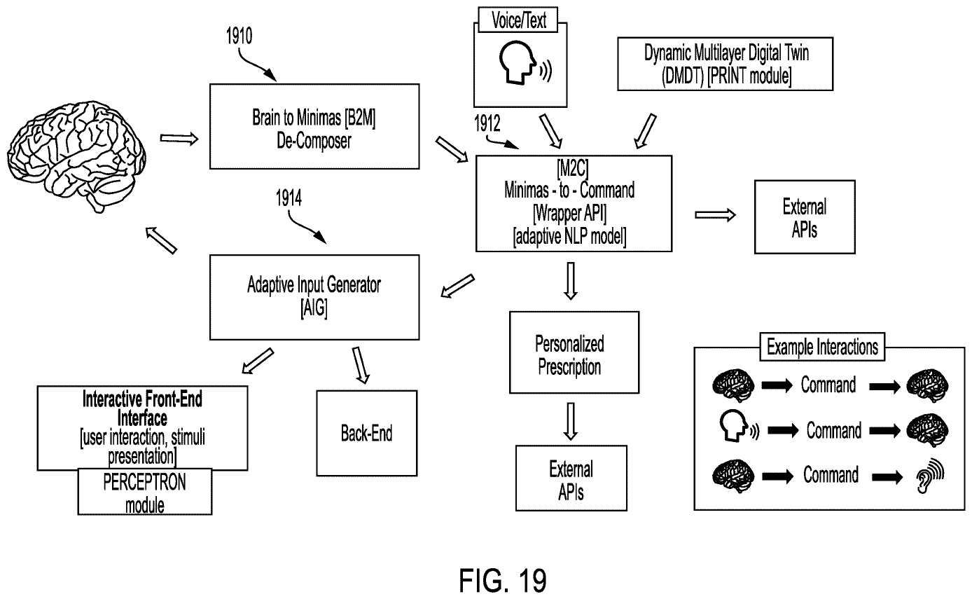

The Brain-To-Command Engine, an example of which is shown in FIGS. 18 and 19, includes three layers, the Brain-to-Minima [B2M] module 1910, the Minima-to-Command [M2C] module 1912, and the Adaptive Input Generator [AIG] module 1914.

The B2M 1910 is an encoder converting patterns of brain activity into commands following a hierarchical structure. The encoder receives raw and processed data from the DMDT of an individual, processes the received data through PREPARE to remove artifacts and noise, and extracts features of brain signals into classes of properties that can be used for multiple applications including, but not limited to, game creation, game design, interactive generative virtual assets creation, collaborative multiplayer asset creation, A.I. control, hardware-control.

The M2C module 1912 transforms minimas from B2M—basic unit of information extracted from brain data—into operational commands used to generate assets and logical language via the AIG module.

The AIG module 1914 allows to generate assets and functions based on commands from M2C. The assets generated include, but are not limited to, 2D and 3D objects and their physical properties; the functions include, but are not limited to, conditional statements, logical operators, and information on time-varying properties of objects. In some embodiments, specific properties of brain signals including, but not limited to, frequency components extracted from a specific brain region or sensor (e.g., spectral power in the theta band) are used to define properties of objects; in some embodiments, properties related to distributed brain activity (e.g., coherence of gamma activity across the entire brain) are used to define higher-level properties, for instance the color of an class of objects (e.g., trees); in some embodiments, network-level properties (e.g., modularity of brain activity across all available regions or sensors) are used to define a relationship between macro elements in the game (e.g., higher modularity=higher number of villages and towns in the map; lower modularity=fewer number); in some embodiments, network-level properties related to efficiency of information transfer (e.g., minimal path length of a brain graph built in a specific frequency band), are used to design the infrastructure organizing a 3D map (e.g., high efficiency=roads connecting villages and towns are efficiently placed to maximize in-game travel; low efficiency=disconnected environment); in some embodiments, the ratio between brain data properties related to different assets and their properties may be combined to created more complex features; in some embodiments, the complexity of a brain signal (e.g. kolmogorov complexity of an EEG time series) is used to define the resolution of in game objects (e.g., high complexity=objects are visualized in 4K video quality).

In some embodiments, the three layers can be integrated with external software packages including, but not limited to, brain computer interface applications to control external hardware and/or software.

In some embodiments, the three layers can be integrated with a natural language processing (NLP) agent to generate commands and to control the generation of complex assets via instructions dictated using voice or text commands, as shown in FIG. 19.

### Neuromorphic Oscillatory Multiscale Adaptive Engine [NOMAD-E]

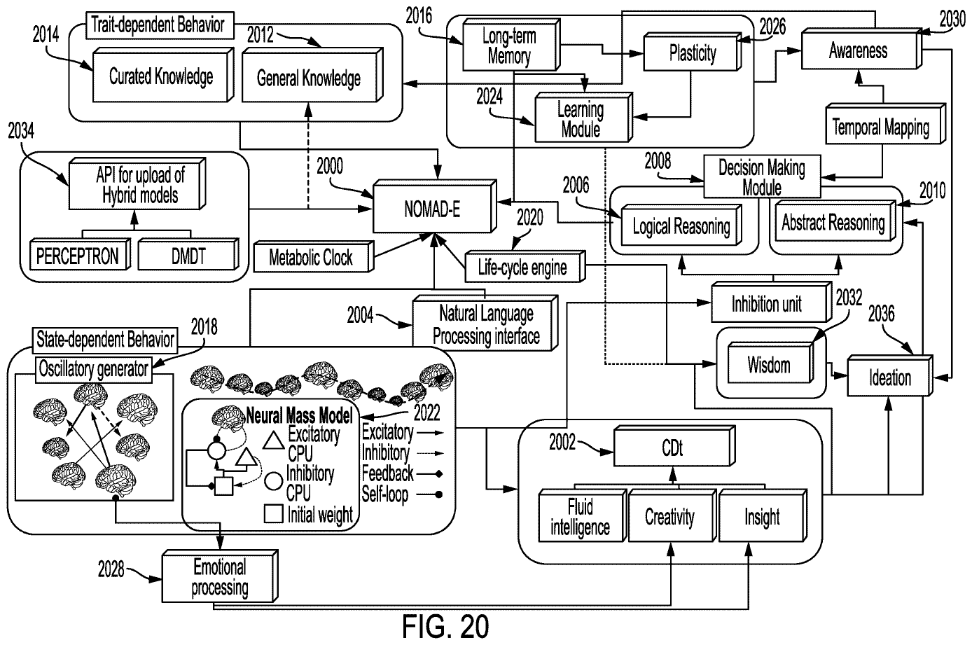

NOMAD-E 2000, an example of which is shown in FIG. 20, is an AGI engine resulting from the combination of computational biology and computational neuroscience principles of micro-scale brain processing, applied to a cognitive architecture for meso-scale and macro-scale processing based on the CDt model 2002 and oscillatory generators, applied to a natural language processing unit 2004 equipped with, but not limited to, long-term and short-term memory capacity, a logical reasoning module 2006, a sentiment analysis core, a decision making module 2008, and an abstract reasoning module 2010.

In some embodiments, the meso-macro scale processing module includes a cluster of independent agents processing information in parallel and providing competition-based emergent decisions. Each agent is informed by a unique set of a-priori information and a set of overlapping, common information shared across agents. Each agent is configured to process new information independently, providing an output weighted on the current mental state of NOMAD-E. The mental state of NOMAD-E is determined by two sub-engines capturing trait and state features of NOMAD-E behavior. Traits of NOMAD-E are stable, core properties of its behavior determined by the conjunction of (i) general knowledge 2012, (ii) curated knowledge 2014 that can be adjusted depending on the specific application, and (iii) long-term memory 2016 including any knowledge of past behavior and its link to specific contexts.

The state of NOMAD-E may be determined by the activity of the oscillatory generator 2018. Each network in the generator represents a subsample of computational units designed to solve for specific problems, by receiving specific inputs and a curated set of information. Each network may have a baseline weight determined by trait properties of NOMAD-E, which include information stored in long-term memory. The baseline weight may determine the influence of the network over the other networks composing the oscillatory generator. The weights may change through time based on a competitive search for energy between the networks. The oscillatory generator may have a fixed metabolic (energy) budget available for each life cycle of NOMAD-E. NOMAD-E may have a life-cycle engine 2020 roughly measuring the passage of time, in arbitrary units. Time may be equated to metabolic budget, with NOMAD-E trying to prioritize efficiency of processing and minimal metabolic consumption rather than speed of processing, while maximizing learning and promoting adaptation. The oscillatory networks may constantly compete for energy, by estimating the optimal pattern of excitatory and inhibitory weights assigned to all the other networks in the generator. This creates a constantly shifting balance of excitation and inhibition between the networks, whose net numerical result is the main output of the engine and the main determinant of brain state, which may also be used to weight on decision making processes.

In some embodiments, the networks composing the oscillatory generator represent cognitive macro-functions including sensory processing, memory-related functions, global attention level, emotional cueing, and/or switching rate. In some embodiments, the networks composing the oscillatory generator can be removed from the generator, augmented, or temporarily silenced.

In some embodiments, the activity of the networks composing the oscillatory generator may be calculated via local neural mass models 2022 composed by different classes of neurons with different functions. The classes of neurons may constitute independent computational units expressing multiple behaviors including, but not limited to, tonic and phasic activation and deactivation, with each neuron being linked to other neurons via connections expressing modulatory activity including, but not limited to, inhibition, excitation, feedforward and feedback loops, noise amplification. Each class of neurons may have a specific oscillatory frequency. The summation of multiple neurons and their connections may generate the dominant oscillatory frequency of a network In some embodiments, the life-cycle engine 2020 may be modulated so that perception of time is accelerated or slowed down, and the metabolic budget is modulated to increase or decrease available energy. The initial weights of the networks in the oscillatory generator may also be modifiable, as well as the information included in long-term memory and the general weights between the different modules composing NOMAD-E. This allows to induce changes in the maturity of the system, with the goal of generating different outcomes and measure the evolution of behavior throughout life-cycles.

In some embodiments, the balance in the oscillatory networks may determine the primary cognitive problem-solving attitude of NOMAD-E at any given time, based on knowledge from CDt 2002. A balancing function between cognitive skills related to fluid intelligence, creativity and insight, may determine the agent's default approach to problem solving, resulting in a more logical, creative or intuitive agent's behavior. An individual using NOMAD-E may be able to modify the agent's default behavior to generate multiple answers to the same problem using different approaches.

In some embodiments, NOMAD-E may include a module representing the summation of information from modules including, but not limited to, the learning 2024 and plasticity 2026 modules, the life cycle engine 2020, CDt 2002, the long-term memory module 2016, the emotional processing module 2028, and the awareness module 2030, to provide an additional decision-making tool based on past experience (wisdom 2032).

In some embodiments, NOMAD-E may be used to generate novel ideas when provided with appropriate inputs. The ideation process may be the result of the activity of the CDt module, the wisdom module, and the awareness module. Ideas including, but not limited to, solutions to formal problems and abstract conceptualizations, may result from the balance between accumulated knowledge by the agent and CDt-related decision making, which includes fluid intelligence, creative and insight-based problem solving In some embodiments, NOMAD-E may be personalized for a given individual, creating a hybrid between general NOMAD-E trait and state features and behavior, and the individual's personality, cognitive architecture, and brain properties. The individual can input their features into an application programming interface (API) 2034, and the different classes of relevant information and their weights may be automatically assigned to corresponding NOMAD-E modules. In some embodiments, NOMAD-E may be personalized for a given individual, creating a hybrid between general NOMAD-E trait and state features and behavior, and the individual's (i) DMDT and (ii) DARWIN. In some embodiments, NOMAD-E may be personalized for a given individual, creating a hybrid between (i) general NOMAD-E trait and state features and behavior, and (ii) real-time brain activity collected via PERCEPTRON.

In some embodiments, NOMAD-E may be used as a cognitive enhancement tool. For instance, the DMDT and DARWIN information of an individual may be uploaded to NOMAD-E, to generate a simulated cognitive model representing an individual's cognitive architecture and dynamics. NOMAD-E may then be used to guide the individual in performing personalized cognitive activities to augment their cognitive performance. NOMAD-E may allow to choose the type of cognitive skills to be augmented and select the most effective cognitive tasks to be performed. Cognitive enhancement may include various cognitive domains and skills including, but not limited to, abstract reasoning, decision making, short-and long-term memory, creativity, attention, perception, language abilities, flexibility, insight, logical reasoning, perceptual reasoning, and/or communication skills. The activities and tasks used to enhance cognitive function include, but are not limited to, topic-specific thinking, philosophical reasoning, logical reasoning, memory tasks and training, problem solving, emotion recognition tasks, ethical problem solving, sustained and divided attention training, and/or mind wandering.

In some embodiments, NOMAD-E may be optimized to generate the highest possible number of novel ideas and insights given a fixed set of baseline information (e.g., IDEATION module). Weights in NOMAD-E oscillatory generator may be set based on brain data collected during idea generation and insight events, where individuals were asked to generate novel solutions to existing problems. The inventor has defined a pattern of brain activity responsible for idea generation, which involves the unbalancing between specific brain networks including, but not limited to, the (i) language, (ii) anterior salience and (iii) executive control networks, and their oscillatory activity. NOMAD-E networks in the oscillatory generator may be programmed to simulate said pattern, and perform a pattern recognition search within available curated and general knowledge databases. NOMAD-E may be programmed to generate answers across multiple spectrums, defined by weights assigned to the emotional processing, wisdom, CDt, awareness and ideation modules. The spectrums may include, but are not limited to, (i) innovation, (ii) level of scientific evidence, (iii) feasibility, (iv) social impact, (v) ethical validity, (vi) scalability, and (vii) protectability (e.g., from existing intellectual property). NOMAD-E may provide a report of each ideas' profile across the spectrum, and an overall score. The user may modify weights of a network and/or module to optimize resulting ideas across the various spectrum.

In some embodiments, the ideation module 2036 may be used to perform research on a particular market segment by providing NOMAD-E with specific curated knowledge. Results of the research may be used to, among other things, find areas of growth for a specific company or entity, identify best strategic partners to promote innovation, and/or generate ideas for novel products. In this embodiment, a product may represent the result of organized thinking towards a goal, leading to a product addressing a need. This includes, but is not limited to, a physical product as in the case of a device or software, and a concept describing the relationship between multiple sources of information. For instance, ideation module 2036 may be used to discover novel hypotheses within a specific scientific field, and to identify candidates for drug repurposing (drugs used for a specific disease that, based on knowledge provided to NOMAD-E, could be efficacious in other conditions based on recent scientific evidence).

### Multimodal Cognition & Plasticity (MCP) Test

In some embodiments, a cognitive test may be used to activate the CD-r. The Multimodal Cognition & Plasticity (MCP) test was composed by the least number of stimuli needed to activate the CD-r, and was composed by audio and video stimuli delivered via a display and an audio device. The test measures the ability of an individual to switch across multiple cognitive domains related to sensory, emotional and cognitive processing, and solving tasks of increasing difficulty with or without time pressure. In some embodiments, the task may ask the individual to solve a memory problem while monitoring the onset of a visual stimuli on the screen, forcing the individual to divide his/her attention while solving a memory problem. Stimuli with emotional valence are then displayed, and the individual is also asked to rate the stimuli based on their emotional activation level. In some embodiments, the individual wears a device for brain data collection while solving the task, and a closed-loop system is used to activate certain aspects of the task in response to changes in brain activity, and vice-versa. In some embodiments, the MCP task is used as a metric of brain plasticity and brain health and may be applied in individuals at risk of developing dementia or other forms of neurodegenerative disorders.

### NEUROMODULATION [STIMOLA]

STIMOLA includes methods allowing to measure and interact with brain function via controlled perturbation. Given the variety of brain states, traits and target configurations, a single brain modifier class is typically not sufficient to elicit the desired effect, and a combined, appropriately timed approach may be more effective in inducing the desired meaningful brain changes.

In some embodiments, STIMOLA may implement a form of noninvasive brain stimulation (NIBS), where electric fields are generated, controlled in space and time, and directed into the brain without the need for any surgical procedure. Examples of NIBS include, but are not limited to, transcranial magnetic stimulation (TMS) in the form of, but not limited to, repetitive TMS (rTMS), patterned rTMS protocols such as Theta Burst stimulation, multi-pulse TMS and paired associative stimulation; transcranial electrical stimulation (tES) in the form of, but not limited to, transcranial direct current stimulation (tDCS), transcranial alternating current stimulation (tACS), and transcranial random noise stimulation (tRNS); and focused ultrasound (FUS).

In some embodiments, TMS and tES may be used to trigger long-term potentiation (LTP) or long-term depression (LTD)-like mechanisms, depending on the specific frequency applied. High-frequency rTMS (>1 Hz) or intermittent TBS-iTBS (e.g., short trains of impulses) often increases cortical excitability and causes LTP-like effects, while low frequency rTMS (≤1 Hz) or continuous TBS-cTBS (e.g., a single train of pulses) more frequently causes a decrease of cortical excitability and eventually LTD-like effects. TMS and tES may be used to modulate GABAergic function related to inhibitory interneurons' activity, and/or modulate the cholinergic system for its role in cognition and cognitive decline.

In some embodiments, noninvasive brain stimulation is performed by targeting Parvalbumin Positive (PV+) inhibitory interneurons in the brain, by means of burst of high-frequency stimulation in the form of magnetic, electrical or ultrasound stimulation. High-Frequency Subcortical Burst (HFSB) stimulation is designed to replicate a thalamic burst of information sent from the thalamus to the neocortex, and may be thought of as being translated into local activity via activation of PV+ interneurons. Stimulation may be performed with a short burst of high frequency stimulation, for instance, but not limited to, tACS at a frequency of approximately 300 Hz sustained for 5 seconds with an inter-burst interval of 2 seconds. Stimulation may be applied directly on the scalp transcranially, via personalized electrodes placement based on DMDT and DARWIN algorithms including biophysical modeling solutions. HFSB may be used to activate PV+ interneurons, with the goal of inducing a modulation of brain neuroinflammatory response, modulate protein clearance and modulate cognitive function by acting on excitation/inhibition balance in the cortex. In some embodiments, stimulation may be delivered via PERCEPTRON and adjusted via closed-loop tools using real-time brain signals. HFSB may be used to activate other classes of interneurons also resonating with thalamic subcortical burst, including somatostatin interneurons. HFSB may have particular applications in the field of neurodegenerative disorders and cognitive enhancement.

### Perturbation Approaches

STIMOLA includes, at least in part, solutions where external electrical, magnetic or ultrasound perturbation is delivered to one brain region at the time, where multiple perturbations are delivered on a single region to assess dynamic brain response, or where perturbation is delivered over multiple brain regions either simultaneously or in a predefined sequence to examine network-level rearrangements of brain dynamics. The method includes, at least in part, solutions where the effect of the perturbation are measured via electrophysiological approaches such as, but not limited to, EEG or EMG.

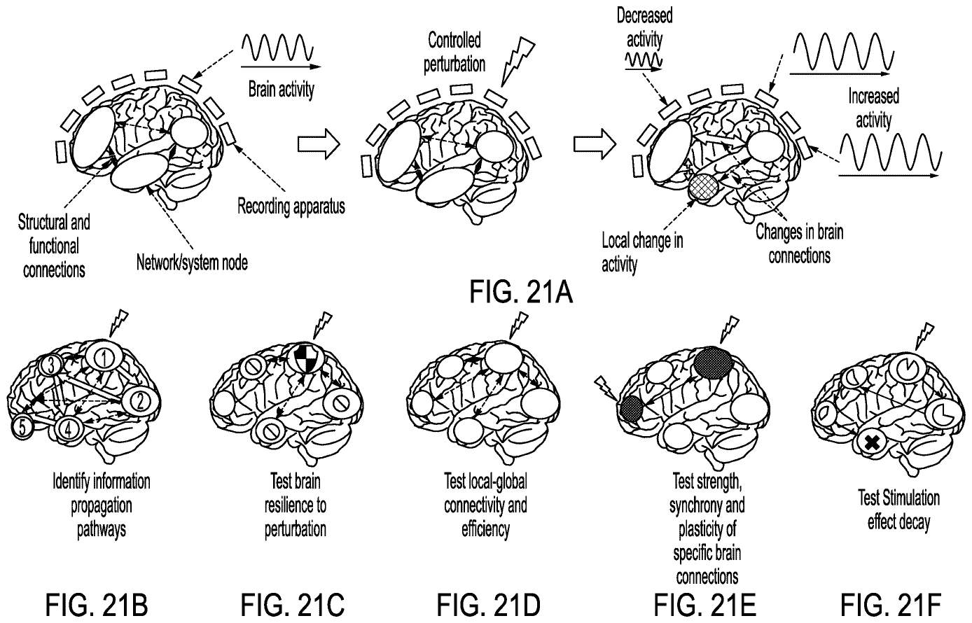

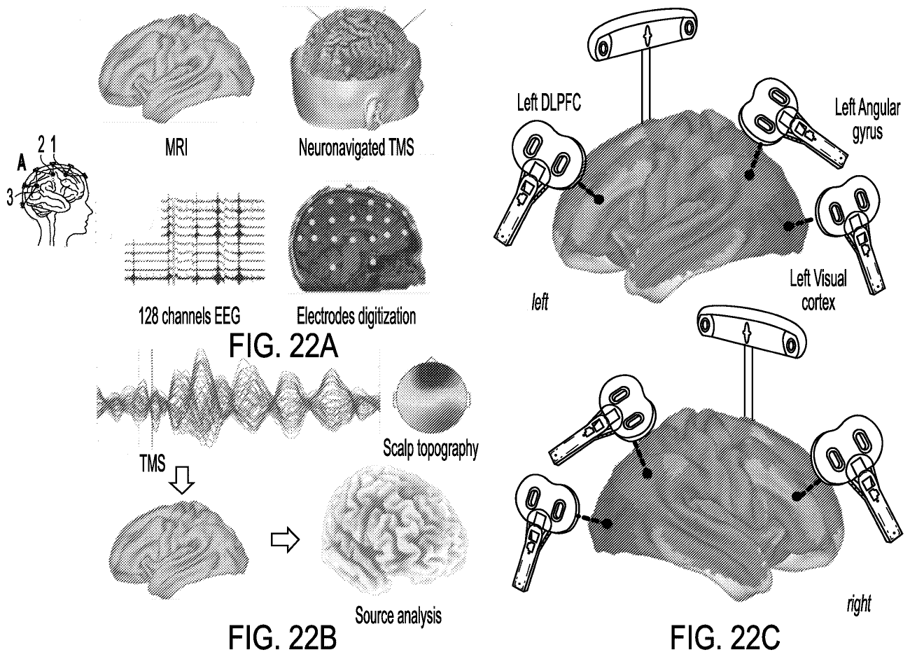

FIGS. 21A-21F show examples of controlled perturbations and brain responses in accordance with some embodiments. Different brain stimulation approaches can be used to perturb brain activity and record responses dependent on individual differences in brain anatomy and function. FIG. 21A shows an example approach that involves collecting brain activity with noninvasive electrodes placed on the scalp, as in the case of EEG, then delivering an electro-magnetic perturbation, as in the case of TMS, and recording the brain's response from multiple regions of the brain, looking, for instance, at an increase in activity following stimulation or an increase in synchronicity between two or more regions or networks. This approach can be used, for example, to measure multiple brain dynamics and to induce specific effects on brain activity.

### Example Perturbation-Based Applications

The perturbation protocols illustrated in FIG. 21A may be used to induce specific changes in brain activity and/or to measure specific features of brain activity characterizing the brain of an individual. FIGS. 21B-21F describe example applications of using such perturbation protocols.

Information processing: FIG. 21B shows that the response to electromagnetic perturbation via TMS can be used to measure the delay of brain response between the region (or regions) targeted by TMS and other regions located with different degrees of proximity from the target regions. Data analysis can provide information on information flow and effective connectivity in the brain, identify regions crucial for information processing and conduction of information, as well as the most efficient pathway to connect two distant brain regions.

Brain Resilience: FIG. 21C shows that analysis of network-level response to electromagnetic perturbation can also be used to calculate brain resilience to perturbation, as a measure of brain's capacity to maintain high efficiency levels in the presence of perturbation mimicking real-life scenario involving, for instance, neurodegeneration of brain tissue, strokes, accumulation of waste products such as amyloid-β and tau protein associated with Alzheimer's disease. Systematic analysis of the amount of “damage/disruption” to brain networks induced by TMS or tCS produces quantitative metrics of brain resilience associated with clinical variables such as cognitive impairment, disease progression rate, brain atrophy.

Brain Efficiency: FIG. 21D shows that targeted application of TMS or tCS during EEG recording can be used to test the strength and efficiency of specific connections in the brain, related to activity within a specific brain functional network, multiple networks, or activity between networks. Metrics related to global brain connectedness may be obtained by looking at brain activity right before and after electromagnetic perturbation.

Brain Plasticity: FIG. 21E shows that stimulation over two or more brain regions via TMS or tCS allows to measure the strength of a particular connection in the brain. By applying repeated TMS/tCS stimulation at specific time intervals, it is possible to induce a change in connectivity strength; the magnitude, speed and duration of such change may be used as a metric of brain plasticity, e.g., the brain's ability to adapt to external perturbation and rearrange its connections. Brain plasticity measures may be used, for example, to estimate a patient's capacity to adapt to brain changes induced by Alzheimer's disease, and therefore estimate trajectories of disease progression and cognitive decline.

Network Degeneration: FIG. 21F shows that perturbation of specific areas and monitoring of signal propagation across other network nodes allows to estimate signal decay in the brain, which can be used as a metric of tissue neurodegeneration caused by, for instance, amyloid-β and tau protein accumulation, atrophy, neuroinflammation and related alteration of white matter tracts. Analysis of both spontaneous and evoked brain activity may be used to estimate regional and global protein load in the brain, using indexes created from EEG data to quantify the amount of waste proteins such as amyloid-β, tau, TDP43 and alpha synuclein.

### Temporal Framework and Scope of Perturbation-Based Markers

The system and methods described herein may be applied for multiple scopes and timeframes. Described below are some applications of perturbation-based markers in patients with Alzheimer's disease and dementia in general.

### Diagnostic Purposes

In some embodiments, individual response to perturbation may be used to identify abnormal brain activity in patients suspected of having Alzheimer's disease or dementia. A response to perturbation dissimilar to a control subject, a healthy control or a previous response obtained from the same individual, may be used to aid the diagnostic process. A similar process may be applied to identify subtypes of Alzheimer's disease within a group of patients diagnosed with Alzheimer's disease (e.g., amnestic Alzheimer). A similar process may be applied to identify individuals with brain cancer at risk of tumor recurrence after surgery. Analysis of the response may be carried out via visual inspection performed by a trained human operator, or via a machine learning-based classification algorithm capable of labeling a response to perturbation as a normal or abnormal response.

### Prognostic Purposes

In some embodiments, analysis of individual brain data may be carried out to predict (e.g., estimate) the course of a pathology (e.g., Alzheimer's disease) based on historical data collected from the same individual or group-level estimates of disease progression. A similar process may be applied to predict the course of pathology in patients with brain cancer, to predict recurrence and estimate survival.

### Evaluation of Treatment Effect

In some embodiments, analysis of individual or group-level brain data may be carried out to quantify differences in brain activity before and after a treatment for Alzheimer's disease or memory problems. Pre-Post treatment changes in perturbation-based metrics may be used to evaluate individual or group-level response to a given treatment, alter the treatment parameters, or facilitate a decision to continue or discontinue treatment.

### Personalized Treatment

In some embodiments, analysis of individual brain data may be carried out to personalize noninvasive brain stimulation parameters including, but not limited to, stimulation location, orientation, intensity, frequency, phase, and/or noise level. Personalization may be carried out by looking at the response to, for instance, TMS pulses delivered over multiple locations within a target region and identifying the location providing the highest brain response to TMS.

### Disease Tracking

In some embodiments, perturbation-based metrics collected over time may be used to monitor brain function in patients with neurological and psychiatric conditions, as well as in healthy individuals, capturing, at any given timepoint, significant deviations of brain activity patterns from data collected at previous timepoints. For instance, individual responses may be compared to normative data collected in a sample of patients with Alzheimer's disease or in a group of healthy controls, thus providing an estimated deviation from, for instance, expected rates of cognitive decline.

### Cognitive Function Assessment

In some embodiments, perturbation-based data may be collected from brain regions/networks associated or supporting specific cognitive functions. For instance, TMS or tCS can be directed towards a region relevant for memory processing, such as the precuneus or the dorsolateral prefrontal cortex. TMS-EEG and tCS-EEG data may be used in combination with neuropsychological scores to identify altered brain circuitry responsible for, for instance, decreased memory performance in patients with Alzheimer's disease.

### Example Applications

In some embodiments, BRAINPRINT and OPTI-BRAIN may be used to simultaneously determine both location and stimulation parameters for NIBS. In some embodiments, tACS may be delivered via a combination of a high frequency carrier frequency in the Megahertz range, and a target frequency in a physiological frequency band. Stimulation within the Megahertz range (>1 Mhz) tends not to induce side effects of canonical tES, such as scalp itching and burning sensation, allowing to increase the intensity of tACS stimulation and therefore reach deeper brain structures or superficial brain regions at a higher intensity. The combination of a high-frequency carrier with a physiological tACS frequency, such as 6 Hz or 40 Hz, allows to deliver higher intensity stimulation and entrain brain activity in a frequency-specific manner. The combination of high-frequency tACS with physiological tACS, may be used to modulate brain activity in the healthy and pathological brain.

In some embodiments, NIBS may be used in combination with BRAINPRINT data to determine location and stimulation parameters for non-invasive brain stimulation. For instance, TMS or tES may be combined with EEG recording, and one or more characteristics of evoked responses to brain stimulation at multiple brain locations may be used to determine the optimal location for targeted NIBS and stimulation characteristics (e.g., stimulation frequency, intensity/amplitude).

In some embodiments, a controlled perturbation is applied to a single brain region, and a response is measured via electrodes placed on the scalp (EEG) or on the body (EMG). Perturbation can have various intensity levels based on a subject brain state including, but not limited to, levels of cortical excitability, plasticity, inhibition, excitation, oscillatory activity, connectivity and/or reactivity. In some embodiments, response to perturbation may be measured via EMG to analyze longitudinal changes in local excitability of a targeted brain system over time after perturbation, as a measure of cortical plasticity. In some embodiments, response to perturbation may be measured via EEG, looking at (a) local responses expressed as, but not limited to, the amplitude, number, frequency and timing of so called TMS-evoked potentials measured in the brain region being stimulated; (b) a distant response measured using the same (or similar) metrics computed from distant brain regions, looking at EEG electrodes or the induced amount of current generated in the brain; (c) a change in interregional dynamics including or not the brain region receiving stimulation, with these measures including, but not being limited to, correlation, connectivity, effective connectivity, dynamic connectivity, graph-theory measures of nodal interactions.

In some embodiments, dual co-localized perturbation may be delivered via a sequence of at least two controlled perturbations repeated over a single region, and a response may be measured via electrodes placed on the scalp (EEG) or on the body (EMG). Response to perturbation may be measured via EMG, looking at longitudinal changes in local excitability within a targeted brain system over time after perturbation, as a measure of cortical plasticity.

In some embodiments, multisite perturbation delivered via a sequence of at least two controlled perturbations may be repeated over multiple distinct brain regions, and a response may be measured via electrodes placed on the scalp (EEG) or on the body (EMG), or via neuroimaging techniques such as MRI. Response to perturbation may be measured via EMG and EEG, looking at longitudinal changes in local excitability within a targeted brain system over time after perturbation, as a measure of cortical plasticity.

In some embodiments, the timing of each pulse delivered in the sequence of at least two controlled perturbations may be defined by looking at the conduction delay between the target brain regions. The delay between the regions may be used to set the delay between the two regions in order to induce a Progressive Associative Stimulation (PAS), strengthening synchronization between the target regions.

In some embodiments, the timing of Progressive Associative Stimulation is calculated by measuring axonal diameter and length of white matter fibers in the brain using diffusion MRI sequences. White matter myelination may be calculated to estimate input propagation velocity in mm/s, and may be combined with information on tracts length to estimate the conduction delay between two or more target regions.

In some embodiments, STIMOLA may be configured to provide a form of stimulation performed to create, induce, modulate or amplify traveling waves recorded from the human brain.

In some embodiments, multimodal perturbation may be delivered via the combination of two or more types of stimulation used to maximize the impact of TMS or tCS on the brain, thereby reducing noise in brain activity data. Oscillatory electrical stimulation (e.g., transcranial alternating current stimulation—tACS) may be used to regulate oscillatory activity in the target region/network before or during the application of TMS. For instance, application of 20 Hz tACS over the precuneus may act as a stabilizer of spontaneous brain activity, for the subsequent delivery of TMS pulses synchronized with the peak of each 20Hz oscillatory cycle.

In some embodiments, brain perturbation may be applied in multiple forms, including, but not limited to, (i) a single pulse, (ii) a train of pulses spaced by a fix interval or a variable jitter, (iii) a continuous waveform characterized by a given amplitude, frequency (or combination of frequencies) and duration, (iv) a continuous electrical field with a given polarity, amplitude and duration, or (v) a signal composed by noise pulsed at a specific frequency.

In some embodiments, perturbation may be delivered to one or multiple brain regions identified based on brain scans and/or electrophysiology data. The sources of information to define optimal perturbation targets can represent population-level data and/or individual data. The data may be organized so that local and distributed brain activity can be summarized in quantifiable metrics; characteristics of brain activity may be extracted, measuring features related to, but not limited to, (a) metabolic and vascular activity, (b) oscillatory activity within known frequency bands, (c) network resilience measures, (d) dynamic connectivity, and/or (e) protein accumulation maps obtained via PET imaging.

In some embodiments, perturbation may be delivered to a node based on its degree of connections with the rest of the brain, to either (i) assess the integrity of the brain connectome or (ii) induce a widespread response in local and distant brain regions as a measure of brain integrity and connectedness. The degree of connectivity may be estimated via functional imaging data or EEG as in the case of functional connectivity, or via anatomical and diffusion MRI scans in the case of structural connectivity. In the latter, the physical connection between two or more regions may be estimated by analyzing the characteristics of white matter fibers (e.g., diameter, anisotropy, diffusivity) composing specific white matter tracts in the brain. In an example scenario, a TMS or tCS stimulation target is defined by examining the structural connectivity of multiple brain regions and then selecting the region with most connections with the rest of the brain, with the goal of maximizing signal propagation and overall stimulation effects. A similar analysis may be performed to identify a brain target with strong structural connectivity with a subcortical brain region not reachable via TMS or tCS (for instance, the hippocampus); stimulation of an accessible grey matter area with strong structural connections with the hippocampus may maximize the probability of indirectly activating the hippocampus as well.

In some embodiments, perturbation may be delivered to two or more nodes of the same brain network, to assess the integrity of connectivity within a given brain network, as in the case of the Default Mode Network in patients with Alzheimer's disease. Targets may be identified via analysis of brain scans (e.g., functional MRI) or electrophysiology data (e.g., EEG data).

In some embodiments, perturbation may be delivered to a node with demonstrated relevance for the pathophysiology of a given disease; for instance, the precuneus region can be targeted for its demonstrated decay of functional connectivity in Alzheimer's Disease, as measured using functional magnetic resonance imaging (fMRI); regions of the temporal lobe affected by waste proteins in Alzheimer's Disease can be a stimulation target, with particular emphasis on amyloid-β and p-tau protein; regions displaying altered level of neuroinflammation as measured via, for instance, by diffusion MRI.

In some embodiments, selection of perturbation targets may be based on estimates of the induced electrical field elicited in a candidate brain region extracted from biophysical modeling of passive tissue conductivity. A model may be created by simulating the propagation of magnetic and electric energy across head and brain tissues (e.g., skin, muscle fibers, bone, cerebrospinal fluid—CSF, grey matter, white matter), resulting in quantitative measurements of induced electromagnetic stimulation affecting brain cells, for instance excitatory neurons and inhibitory interneurons. The magnitude of induced electromagnetic stimulation may be used to adjust stimulation parameters such as intensity and phase angle, to maximize stimulation effects on given brain target(s) according to known standards for inducing, for instance, neuronal firing or the release of neurotransmitters.

In some embodiments, the target region may be identified as a region responsible for a specific cognitive process, for instance long-term memory or attention. Brain scans or EEG data collected during the execution of a cognitive task by a patient with a neurological or psychiatric condition may be analyzed, identifying regions whose activation is related to performance at the task. In an example scenario, one or more resulting regions may then be selected as target for TMS or tCS perturbation to evaluate their level of integration with the other brain regions involved with the same cognitive process (e.g., long-term memory), as a measure of efficiency within the long-term memory network in patients.

In some embodiments, the target region(s) may be identified based on a spatial and functional search algorithm, where, for example, evoked EEG activity after at least 2 TMS pulses is averaged, revealing TEPs at different latencies between 5 ms and 500 ms after TMS. The amplitude of the TEPs may be calculated for each patient and used as a proxy of individual responsiveness to TMS and therefore as an index of the target region/network's excitability and reactivity. The amplitude of TEPs may then be used to correct the stimulation intensity obtained from stimulation of the motor cortex (e.g., resting motor threshold (RMT)), with the goal of adapting TMS stimulation intensity based on TEPs.

The intensity of stimulation for TMS, tCS and tFUS stimulation depends on individual brain anatomy and can be estimated by creating high-resolution biophysical models of brain anatomy via MRI scans. In some embodiments, the electric field (E-field) induced over the brain target may be generated by using a realistic volume conductor head model generated based on MRI images and segmentation from a validation dataset. The model may be based on anisotropic conductivity values for each brain tissue class (e.g., skin, fat, muscle, bone, cerebrospinal fluid, grey matter, white matter) expressed in S/m. The set of resulting meshes, comprehensive of at least gray and white matter, scalp, bone, and cerebrospinal fluid, may be used to calculate the E-field distribution for a specific TMS coil design, position, angle and rotation, accounting for coil-to-scalp distance and brain atrophy. The estimated E-field can be used to (a) retrospectively calculate individual differences in the amount of current delivered over a target region and therefore to explain differences in response to a treatment, or (b) to adjust stimulation location and/or intensity so that all participants receive the same amount of induced cortical stimulation.

In some embodiments, STIMOLA may be configured to provide a form of stimulation performed in a VR or AR environment. The sections describing NEUROCREATOR and the Metaverse herein provide additional details.

In some embodiments, STIMOLA may be used in combination with drugs acting on neuroplasticity, in particular those affecting the PNN. The section describing SYNAPSE herein provides additional details.

In some embodiments, STIMOLA may be applied for diagnostic purposes. Individual response to perturbation may be used to identify abnormal brain activity in patients suspected of having Alzheimer's disease or dementia. A response to perturbation dissimilar to a control subject, a healthy control or a previous response obtained from the same individual, may be used to aid the diagnostic process. A similar process may be applied to identify subtypes of Alzheimer's disease within a group of patients diagnosed with Alzheimer's disease (e.g., amnestic Alzheimer). Analysis of the response may be carried out via visual inspection performed by a trained human operator, or via a machine learning-based classification algorithm capable of labeling a response to perturbation as a normal or abnormal response.

In some embodiments, STIMOLA may be applied for prognostic purposes. Analysis of individual brain data may be carried out to predict (e.g., estimate) the course of a pathology (e.g., Alzheimer's disease) based on historical data collected from the same individual or group-level estimates of disease progression.

In some embodiments, STIMOLA may be applied to evaluate treatment effects. Analysis of individual or group-level brain data may be carried out to quantify differences in brain activity before and after a treatment for Alzheimer's disease or memory problems. Pre-Post treatment changes in perturbation-based metrics may be used to evaluate individual or group-level response to a given treatment, alter the treatment parameters, continue or discontinue treatment.

In some embodiments, STIMOLA may be applied for the definition of personalized treatment regimes and/or parameters. Analysis of individual brain data may be carried out to personalize noninvasive brain stimulation parameters including, but not limited to, stimulation location, orientation, intensity, frequency, phase, and/or noise level. Personalization may be carried out by looking at the response to, for instance, TMS pulses delivered over multiple locations within a target region and identifying the location providing the highest brain response to TMS.

In some embodiments, STIMOLA may be applied for disease tracking. Perturbation-based metrics collected over time may be used to monitor brain function in patients with Alzheimer's disease, capturing, at any given timepoint, significant deviations of brain activity patterns from data collected at previous timepoints. Individual response may be compared to normative data collected in a sample of patients with Alzheimer's disease or in a group of healthy controls, thus providing an estimated deviation from, for instance, expected rates of cognitive decline.

In some embodiments, STIMOLA may be applied for investigation of cognitive function. Perturbation-based data may be collected from brain regions/networks associated or supporting specific cognitive functions. For instance, TMS or tCS can be directed towards a region relevant for memory processing, such as the precuneus or the dorsolateral prefrontal cortex. TMS-EEG and tCS-EEG data may be used in combination with neuropsychological scores to identify altered brain circuitry responsible for, for instance, decreased memory performance in patients with Alzheimer's Disease.

In some embodiments, STIMOLA may be applied for sleep modulation, by acting on brain oscillatory activity associated with, but not limited to, a brain state transition from wakefulness to sleep and a specific sleep stage including but not limited to non-rapid eye movement (REM) and REM sleep stages. STIMOLA may be used in combination with DMDT and DARWIN to select the optimal stimulation frequency, intensity, duration and location for a given individual, in order to maximize the response to stimulation. In some embodiments, STIMOLA may be applied during sleep to increase the duration and representation of specific sleep stages associated with protein clearance and activity of the lymphatic system, with the goal of increasing protein clearance in the brain and remove waste products associated with cognitive decline and pathological aging including but not limited to amyloid-β, tau protein, alpha synuclein and TDP43 protein. STIMOLA may be applied with the intent of protein clearance in individuals including healthy cognitively intact individuals, those at risk of developing dementia, those with preclinical dementia, and those with a diagnosis of dementia including Alzheimer's Disease. In some embodiments, stimulation may be performed using transcranial electrical stimulation in a specific frequency band, including but not limited to the theta frequency band between 3 Hz and 7 Hz. In some embodiments, electrical stimulation may be modulated by ongoing EEG data monitoring and by the phase of the breathing and/or cardiac cycle, in phase with CSF production in the brain. According to state-to-state transition principles described in DARWIN, electrical stimulation may be modulated in real-time on the basis of EEG data capturing different sleep stages and the stability of each stage, identifying transition periods by looking at the ratio of specific oscillatory dynamics including, but not limited to, burst of gamma activity within 30 Hz and 150 Hz.

In some embodiments, STIMOLA may be used to modulate sleep patterns monitored via EEG using PERCEPTRON and DARWIN, to increase sleep efficiency by stabilizing the duration and complexity of brain activity in specific sleep stages including but not limited to REM and non-REM stages. STIMOLA may be used to increase sleep efficiency, stabilize circadian rhythms and enhance memory consolidation and retrieval, in individuals including healthy cognitively intact individuals, those at risk of developing dementia, those with preclinical dementia, and those with a diagnosis of dementia including Alzheimer's Disease. In some embodiments, the impact of STIMOLA for sleep modulation may be evaluated with DMDT measures related to circadian activity and behavioral patterns collected with wearables such as activity watches and PERCEPTRON.

### EXAMPLES

Some embodiments described herein are further illustrated by the following examples and detailed protocols. However, the examples are merely intended to illustrate embodiments and are not to be construed to limit the scope of the technology described herein. The examples described below demonstrate the application of perturbation-based methods to (i) predict individual cognitive status (ii) assess brain's resilience to perturbation, and (iii) measure brain oscillatory activity, plasticity and neuroinflammation levels by means of EEG response to tCS in AD patients.

### Differences in Response to Perturbation and Relationship with Cognitive Profile

Individual response to electromagnetic perturbation can be used to unveil individual brain properties relevant to cognitive processing, and therefore identify subtle changes in the cognitive profile of patients with Alzheimer's Disease (AD) and related dementias. Perturbation via TMS can be delivered over a selection of brain regions known for their role in specific cognitive processes, or known to be affected by the pathology, and their response may be quantified and correlated with individual scores at cognitive/neuropsychological tasks addressing functions such as memory, attention, language, abstract reasoning. For instance, the amplitude of a local response in a given brain region might correlate with working memory abilities, whereas the synchronicity between two nodes of a brain network associated with attention might explain individual variability in executive functions such as inhibition and flexibility. Given the possibility to collect this information “passively,” e.g., without having the subject actively engage in a cognitive test or a particular task, these methods are particularly useful in patient populations characterized by cognitive impairment, behavioral disturbances and lack of compliance (as in the case of AD patients). Moreover, these methods for identification of altered cognitive function via passive perturbation also reduce patients' burden by potentially adding valuable information on a patient's cognitive status without the need for extensive and tiring cognitive assessments.

Below data collected on a sample of patients with Alzheimer's disease is reported. The patients underwent a TMS-EEG session where multiple brain networks were stimulated with the explicit goal of identifying correlation with specific cognitive scores also collected on the same group of patients. Participants also underwent MRI scanning for TMS target selection, following the procedures described herein.

### Methods and Analysis

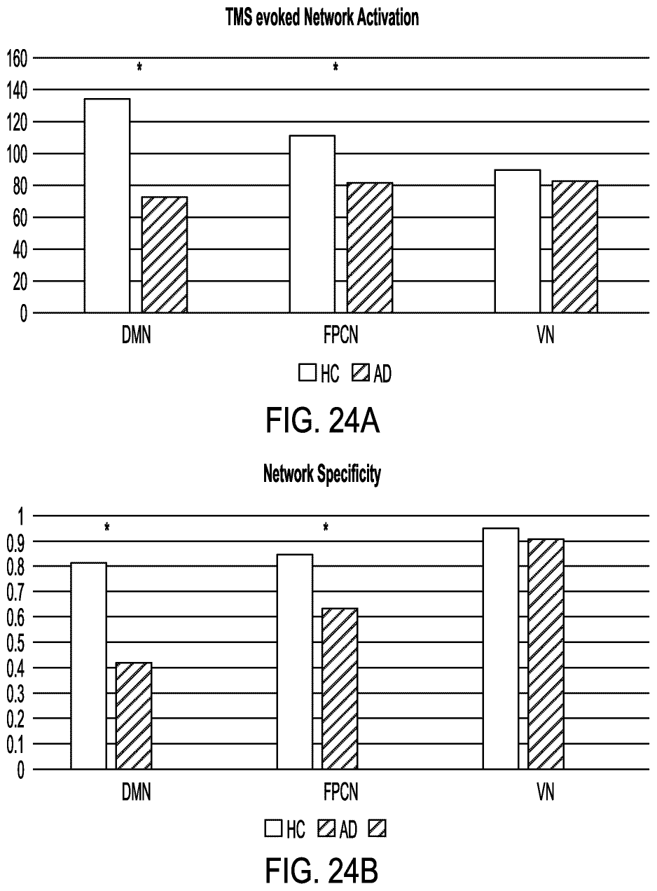

TMS was delivered over multiple networks of the brain based on individual MRI and fMRI data collected in patients with Alzheimer's disease as shown in FIGS. 24A-24C. Specifically, stimulation was targeted towards the angular gyrus as a node of the Default Mode Network (DMN), the functional network responsible for memory processing and mostly affected by pathology in patients with Alzheimer's disease; the Frontoparietal Control Network (FPCN), for its role in high-order cognitive function; and the Visual Network (VN) as a control network where disruption of connectivity has not been reported in patients with Alzheimer's Disease.

Batches of single TMS pulses were delivered over each region (120 pulses each), under neuronavigation monitoring. A 128 channel EEG device was used to monitor brain activity before, during and after each TMS pulse. TMS intensity was set based on resting-motor threshold values collected over the primary motor cortex. EEG data was processed following the pipeline described in PREPARE, resulting in TMS-Evoked potentials (TEPs) that were subsequently projected into each patient's MRI scan to perform source-level analysis of network activity. Patients' cognitive profile was characterized via standard cognitive tools, and a composite cognitive score were created. The composite score allowed to split the sample in High and Low cognitive composite score, therefore looking at TEPs differences across the two groups that might explain different cognitive profiles and rates of cognitive decline.

### Results

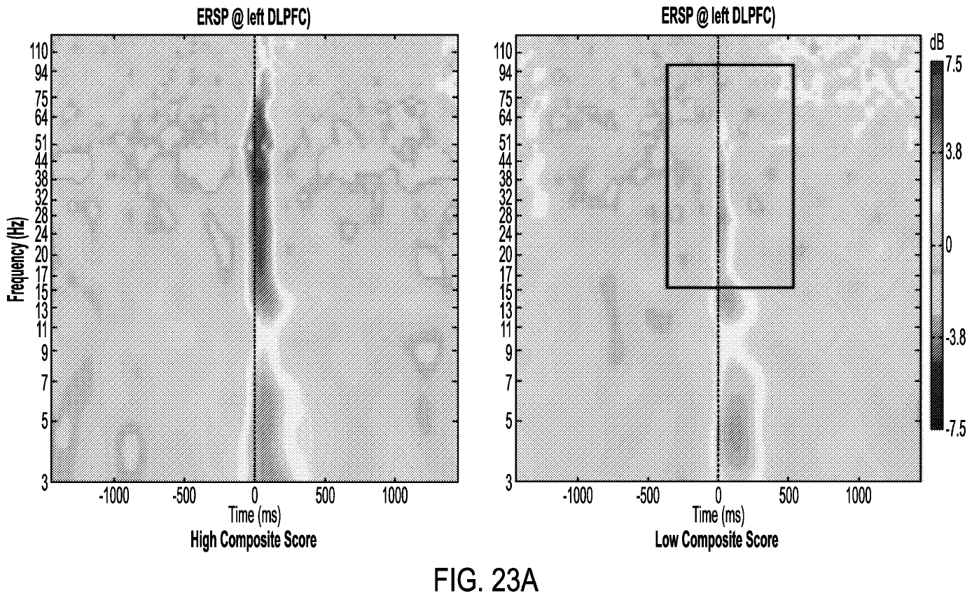

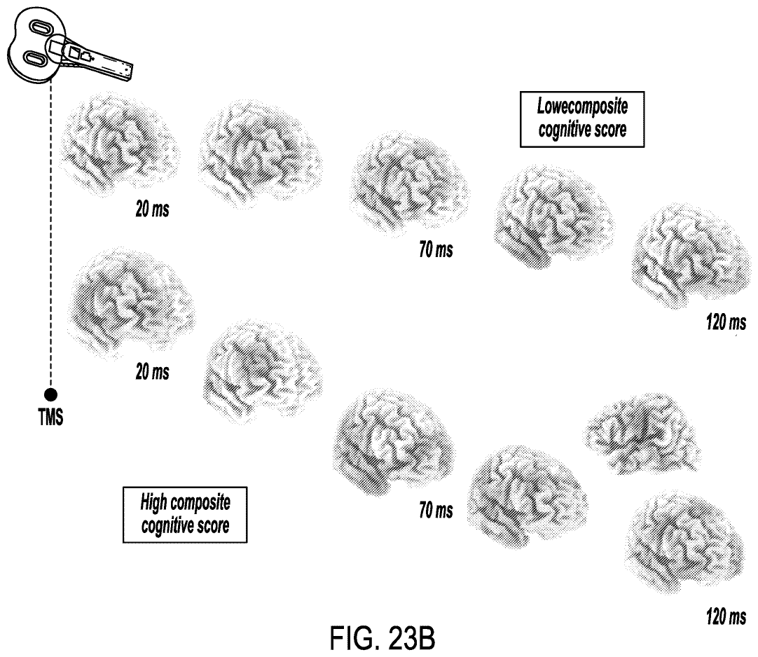

FIGS. 23A-23B show evoked oscillatory activity in Alzheimer's patients after TMS. FIG. 23A shows differences in brain response to stimulation of the dorsolateral prefrontal cortex, with stronger high-frequency activity right after perturbation in patients with higher composite cognitive scores vs low composite cognitive scores. FIG. 23B represents a source-level analysis of TMS-EEG data and shows how the TMS-induced signal propagates more across the brain after perturbation in patients with higher composite cognitive score compared to those with lower composite score where stimulation only induces a local response. This reflects alterations of brain connectivity and functional integrity preventing signal propagation in the brain of patients with more pronounced cognitive decline.

Patients with high composite cognitive score displayed a stronger response to TMS delivered over the DMN and FPCN, while no differences were noted with regard to the VN. In particular, when patients were stimulated over the left dorsolateral prefrontal cortex (DLPFC), a significant increase in fast oscillatory activity was observed (in the gamma band), which was more pronounced in the group with high composite cognitive score compared to low composite cognitive score (p.<0.002), as shown in FIG. 23A. Topographical analysis of signal propagation in the brain also revealed a difference in the magnitude of activation throughout the brain after TMS of the DLPFC, with individuals with higher composite scores displaying a more distributed response as compared to those with low composite scores, as shown in FIG. 23B.

FIGS. 24A-24B show network-level response to perturbation. FIG. 24A shows that patients display reduced activation in networks related to cognitive function and affected by Alzheimer's disease, such as the DMN and FPCN, whereas no significant differences were observed in the VN. FIG. 24B shows that the specificity of the TMS-evoked response, as measured via a ratio between the response in the targeted network and the response in any other network of the brain, also showed significant differences across groups for DMN and FPCN, but not VN.

A comparison between the activation levels for the three networks in patients vs controls also revealed a general reduced response for DMN and FPCN, with no significant differences observed for VN, as shown in FIG. 24A. Moreover, to identify differences in the specificity of the TMS-evoked response across the brain, a comparison was performed by analyzing the amount of activation induced in the stimulated network (e.g., DMN) and activation in other networks of the brain. Results showed a significantly less specific response in patients vs controls regarding the activity in the targeted network, indicating a more distributed, unspecific, “disorganized” response in patients with Alzheimer's disease, reflecting a disruption of network integrity, as shown in FIG. 24B.

In this example, it was shown that a TMS-EEG investigation of network dynamics in patients with Alzheimer's disease may be a valuable tool to identify alterations of brain function relevant to understand individual differences in cognitive performance and cognitive decline in patients with Alzheimer's disease.

### Response to Frequency-Specific Electrical Perturbation in Ad Patients

Alzheimer's disease (AD) is characterized by impaired inhibitory circuitry and GABAergic dysfunction, leading to reduced fast brain oscillations in the gamma band (γ, 30-90 Hz). Assessment of such activity could lead to the identification of diagnostic and/or prognostic biomarkers. In this example, a multimodal “Perturbation-based” transcranial Alternating Current Stimulation (tACS)-EEG protocol to detect γ alterations in patients with AD was tested, and individual responses to tACS were correlated with clinical phenotype.

### Methods and Materials

Participants with mild to moderate AD (mean age=72) underwent a multimodal assessment including tACS-EEG, cognitive testing, blood markers, and Theta-Burst TMS to assess (LTP)-like brain plasticity. The tACS-EEG visit included brief (6′) tACS blocks at 40 Hz or 6 Hz over 6 locations covering 4 lobes in both hemispheres, with pre/post 32-Channels EEG recording for each block. Spectral power in 4 frequency bands was calculated for pre-post EEG. Pre-Post spectral power changes (delta values) were calculated as a metric of gamma induction and correlated with cognitive scores, blood markers and brain plasticity measures.

### Results

Regarding cognitive function and gamma induction, 40Hz-tACS-and 6Hz-tACS-induced y activity correlated with measures of cognitive and memory impairment, like episodic memory (r=0.74), global cognition (r=0.78), language expression (r=0.74) (all ps<0.01), auditory learning skills (r=0.81, p<0.05). Regarding gamma induction and neuroinflammation markers, 40 Hz-induced brain activity correlated with cytokines, tumor necrosis factor-α (TNF-α) (r=−0.73), Interleukin-10 (IL-10) (r=0.77) (all p.<0.01), respectively. Regarding, gamma induction and cortical plasticity measures, cortical plasticity correlated with 40 Hz-induced brain activity (r=0.69, p<0.05) The results of this example provide evidence supporting the use of perturbation-based EEG markers combined with brain stimulation in Alzheimer's Disease (AD), for the detection of clinical and cognitive correlates of the disease, as well as to estimate levels of neuroinflammation in the brain of patients with AD and cortical plasticity levels.

### NEUROPLASTICITY [SYNAPSE]

Some of the systems and methods described herein relate to applications for modulating aspects of brain plasticity and neuroplasticity as part of SYNAPSE, including but not limited to:
(i) a platform for combined interventions leveraging global brain plasticity increase obtained via (a) interventions modulating the Perineuronal Net (PNN) and Extracellular Matrix (ECM), including but not limited to drugs and physical exercise, and (b) Local/Targeted modifications induced via external modifiers including noninvasive brain stimulation methods; and
(ii) a platform for Simulated & Accelerated Enriched Environment via targeted, systematic stimulation of brain function using noninvasive brain stimulation and other brain modifiers including cognitive stimulation and virtual reality.

Brain development and performance generates from the synergistic action of both genetic factors and factors related to experience. The interaction between the central nervous system, the extracellular environment (e.g., physical space surrounding brain cells) and the signals arising from the external world (e.g., experience) determines the adaptation of the brain to external inputs coming from the environment and promote its maturation. The concept of brain plasticity represents the capability of the brain to modify its structure and function in relation to the experience. Brain plasticity may be achieved via the fine regulation of growth-promoting and growth-inhibitory signals. In the post-natal brain, many processes involved with brain development are highly active, e.g., synaptogenesis processes, maturation of the glia, regulation of neuronal growth, axonal pruning take place. Plasticity levels are at the highest level and rapidly decline over the subsequent 4-5 years. The neonatal brain is highly responsive to external stimulation, can easily adapt its connection, resulting in a high capacity for learning and adaptation (e.g., learning language, how to walk). The end of the critical period defines a drastic change in the brain's ability to adapt, learn and evolve, making the quest for solutions able to re-open the window of plasticity a key unmet need in modern neuroscience, with applications including but not limited to cognitive enhancement, physical and cognitive rehabilitation, and accelerated learning. Some of the systems and methods described herein include tools and protocols to re-activate plasticity in the healthy and diseased adult brain, to enhance normal brain function and/or treat brain disease.

The SYNAPSE module may include behavioral, cognitive and noninvasive brain stimulation methods to modulate, enhance and reactivate brain plasticity levels via modulation of the extracellular matrix (ECM), the perineuronal net (PNN), chondroitin sulphate proteoglycans (CSPGs) and inhibitory interneurons. During the critical period, the ECM supports neurogenesis, synaptogenesis, cell migration, growth, and axonal elongation, whereas in the adult brain it is involved with neuronal plasticity and regeneration following damage. The main components of the ECM are CSPGs, proteoglycans mainly exerting growth-inhibitory roles and therefore preventing cellular adaptation and plasticity. The PNN constitutes a portion of pericellular matrix that wraps around the soma and dendrites of neurons in the brain and central nervous system more in general. The PNN provides structural support while also limiting growth and inhibiting plasticity. ECM molecules and PNN preferentially accumulate around specific classes of neurons, e.g., inhibitory interneurons. Among these, parvalbumin expressing interneurons (responsible for activating microglia in the presence of viral infection or neurodegenerative diseases such as Alzheimer's disease) are particularly affected by abundant PNN and ECM composites.

In some embodiments, noninvasive brain stimulation approaches as described in the STIMOLA section herein may be used to induce a state of increased neuroplasticity including, but not limited to, transcranial magnetic stimulation (TMS), repetitive TMS (rTMS), patterned rTMS protocols such as Theta Burst stimulation, multi-pulse TMS and paired associative stimulation; transcranial electrical stimulation (tES) in the form of, but not limited to, transcranial direct current stimulation (tDCS), transcranial alternating current stimulation (tACS), transcranial random noise stimulation (tRNS); and focused ultrasound (FUS).

In some embodiments, frequency-specific neuromodulation targeting the activity of inhibitory interneurons may be used to modulate the activity of interneurons and elicit a modulation of the PNN. Given the link between inhibitory activity of interneurons and oscillatory patterns in the gamma band (between 30 Hz and 120 Hz), frequency-specific stimulation in the gamma band may be used to target brain systems/networks/regions. Stimulation may come in the form of, but is not limited to, sinusoidal stimulation, patterned stimulation (e.g., burst of white noise imposed on a carrier frequency at 40 Hz), or multi-frequency cross-frequency stimulation.

In some embodiments, brain optimization and evolution algorithms (e.g., as described in the DARWIN, SYNAPSE, IMPROVE, OPTI-BRAIN and OPTI-COG sections herein) may be coupled with other interventions able to promote brain plasticity and therefore accelerate/reinforce brain changes.

In some embodiments, interventions configured to promote brain plasticity may include drugs able to disrupt the PNN and re-open the window of plasticity in the brain including, but not limited to, those acting on growth-promoting and growth-inhibiting factors, as well as on specific extracellular matrix molecules, such as chondroitin sulfate proteoglycans (CSPGs), hyaluronan (HA) tenascin-R, and link proteins, as well as drugs acting on GABAergic activity, antidepressant, ketamine, drugs acting on Brain Derived Neurotropic Factor (BDNF). Candidate drugs include, but are not limited to, those based on chABC, an enzyme able to degrade CSPGs molecules and reactivate the critical period, Ketamine for its ability to induce dissociative states, opioids, psychoactive drugs, psychedelic compounds capable of robustly promoting neuritogenesis including, but not limited to, tryptamines (N,N-dimethyltryptamine-DMT-, and psilocin), amphetamines (2,5-dimethoxy-4-iodoamphetamine-DOI- and 3,4-methylenedioxy-methamphetamine MDMA), and ergolines (lysergic acid diethylamide-LSD).

In some embodiments, a drug able to temporarily disrupt the PNN may be used to increase plasticity at the whole-brain level; OPTI-BRAIN may be used to identify targets and parameters for brain optimization, including regions and/or connections where a further boost of local plasticity might be desired; STIMOLA may be used to further enhance plasticity on a given brain target.

In some embodiments, a drug able to temporarily disrupt the PNN may be used to increase plasticity at the whole-brain level; once increased plasticity is achieved, OPTI-BRAIN may be used to identify regions and/or connections where STIMOLA may be used to selectively modify specific brain connections, networks, regions, to reach a desired outcome (e.g., change of state or trait).

In some embodiments, physical activity may be used to modulate the PNN and promote a state of increased brain plasticity. A subject may be asked to perform an aerobic physical task (e.g., running or walking on a treadmill) to increase brain metabolic activity and promote brain plasticity, while also performing a more specific task loading on a specific brain system/network/region (e.g., a working memory task to activate the dorsolateral prefrontal cortex—DLPFC).

In some embodiments, sensory deprivation protocols may be used to elicit an increase in brain plasticity in a specific system/network/region of the brain. A subject may be asked to complete a visual task while wearing an eye-patch covering one eye, inducing a state of distress in the visual system which triggers a protective response leading to increased plasticity (simulated deficit protocol). The same approach may be used in the auditory and motor system, and may be combined with specific training protocols to increase plasticity and enhance behavioral performance (e.g., sensory deprivation on the right arm to induce neuroplasticity and increase motor learning).

In some embodiments, disruption of the PNN may be used to affect long-term memory. The integrity and configuration of the PNN has been shown to be important for maintenance of long-term memories, with its disruption leading to loss of long-term memory. SYNAPSE and STIMOLA may be used to target traumatic long-term memories via disruption of the PNN in areas relevant for traumatic memories and post-traumatic stress disorder, including but not limited to the amygdala, the hippocampus, the precuneus, and anterior cingulate cortex, the insula.

In some embodiments, SYNAPSE may be used to create a virtual augmented environment (VAE) with the goal to promote neuroplasticity. VAE refers to a virtual, augmented or mixed reality environmental maximizing sensory, motor, cognitive, and social stimulation, resulting in increased stimulation of brain cells (including but not limited to synaptic remodeling, dendritic growth to gliogenesis, angiogenesis and neurogenesis) and systems, promoting adaptation and plasticity. The concept of enriched environment (EE) is used in animal research to promote learning, recovery from injury and plasticity via modulation of the expression of several neuronal growth-genes, including but not limited to changes in neurotrophins expression. The VAE is a parametric environment where stimuli and their properties are selected via an algorithm (Plasticity Maximization Algorithm—PMA) based on, but not limited to, physical properties of 3D objects (e.g., complexity, color, shape), their interaction capability with human agents, their affective value, and their effect on brain structures (including but not limited to increase in vascular flow, metabolism and connectivity). Stimuli may be organized based on their impact on plasticity and combined in specific ensembles to create optimal plasticity-inducing stimulation according to individual brain properties as defined via DMDT.

In some embodiments, the PMA may be used to integrate elements of a VAE into a videogame experience, for seamless exposure to passive plasticity-inducing stimulation during engagement in entertainment activities. Elements composing the background, active elements in the foreground or interactive elements in a game may be designed to include characteristics inducing plasticity, thus exposing players to a constant plasticity-inducing experience.

In some embodiments, a VR environment may be created to elicit a specific brain state where stimuli are visualized in specific sequences to elicit a brain response representative of a more mindful, calm state, with decreased activation of emotion-regulation areas of the brain such as the amygdala, thus favoring neutral reprocessing of traumatic memories. Stimuli are visualized, for instance, as alternating between the four quadrants of the visual field, with predefined trajectories along the horizontal and vertical axis. The induced brain activation simulates a pattern of visual exploration evolutionary associated with focus towards external activity and decreased mind wandering, inner dialogue and emotional activation. The Metaverse section herein provides additional explanation and examples.

In some embodiments, a VR environment may be created to elicit a parametric manipulation of sensory perception with the goal to perturb sensory systems of the brain and induce plasticity. These systems include, but are not limited to, the visual, somatosensory, motor and auditory systems. Applications include, but are not limited to, exposure to altered sensory stimuli mimicking an alteration of function in a sensory cortex of the brain including, but not limited to, a change in the visual field so that objects are, for instance, being visualized in a spatial location different then their actual physical location in the VR environment. In this specific example, an object may be visualized at a certain degree of mismatch between what is perceived from the visual system and what is visualized in the 3D environment; the individual exposed to such stimulation may be progressively trained to adapt to the spatial mismatch and correct his/her visuomotor plan in order to reach the 3D object in its correct location (e.g., grab an object by pointing at a location 30 degrees to the left of the actual object; systematically process this visuo-motor adaptation for every object in the environment). This process of adaptation may induce a remodeling of synaptic connectivity in the visual cortex and in other cortices of the brain and the cerebellum, which may lead to an increase in brain plasticity as measured via increased levels of Brain Derived Neurotrophic Factor (BDNF) and other brain markers. After the training, heightened levels of plasticity can be used as a primer for other brain, cognitive and behavioral manipulations including, but not limited to, cognitive training, behavioral therapy, and physical training. After the training, the sensory cortex (e.g., the visual cortex) may slowly readjust to stimuli displayed in their correct location and normal synaptic connections may be restored. This intervention, called Virtual Sensory Manipulation (VSM), can be applied to other sensory systems as well following the same principles, for instance inducing a mismatch in the spatial location of a 3D audio source compared to the actual location of a speaker displayed as a 3D object in a VR environment; the same may apply to tactile stimulation when devices providing vibrotactile feedback are used in a VR environment. The VSM can be embedded in different media using VR, including existing and ad-hoc videogames. In some embodiments, the VSM is used to retrain and perturb cognitive systems as well, by embedding cognitive tasks and decision making processes in the sensory retraining process; for instance, an user is asked to recalibrate his/her grasping movements by 10, 20 and then 30 degrees for objects in the 3D environment, but only for objects colored in red; in another example, objects in blue require an adjustment of 10 degrees to the left, whereas objects in green require a 30 degrees adjustment to the right; these additional constraints and rules induce an engagement of cognitive functions such as attention, working memory, inhibition and flexibility relevant for brain health and healthy aging.

In some embodiments, the Enriched Environment (EE) elicits changes in brain structure and function relevant for plasticity, learning and brain optimization. These changes can be represented by their location and/or magnitude in the brain. For instance, exposure to EE with high dimensionality of color-properties might elicit higher activations and plasticity in brain areas related to vision, such as the occipital lobe of the brain. SYNAPSE may be used to identify such brain targets and bypass the need for physical or simulated stimulation by means of direct stimulation of the brain via noninvasive brain stimulation using STIMOLA.

In some embodiments, a brain state generated by EE may be synthesized and corresponding brain targets are defined via OPTI-BRAIN and OPTI-COG; STIMOLA may then be used to stimulate said targets using methods such as, but not limited to, transcranial electrical stimulation, transcranial magnetic stimulation, or transcranial ultrasound stimulation.

In some embodiments, SYNAPSE solutions for brain plasticity enhancement may be used for the treatment of Alzheimer's disease (AD) and other conditions characterized by alteration of brain plasticity mechanisms, altered inhibitory interneurons' activity, decrease of fast oscillatory brain activity and alterations of brain network dynamics. AD is characterized by diffuse amyloid-β (Aβ) plaques and phosphorylated tau (p-tau) deposition in neurofibrillary tangles, as well as widespread neurodegeneration, signs of neuroinflammation and altered plasticity processes in the brain generating from altered activity of inhibitory interneurons.

In some embodiments, DMDT and DARWIN may be used to derive personalized interventions to boost brain plasticity in patients with AD; STIMOLA can be used to deliver noninvasive brain stimulation solutions aimed at enhancing activity of interneurons activity, disrupting the PNN and enhancing plasticity.

### EXAMPLE APPLICATIONS

Some embodiments described herein are further illustrated by the following examples and detailed protocols. However, the examples are merely intended to illustrate embodiments and are not to be construed to limit the scope of the technology described herein.

### Combined Synergistic Application of Plasticity-Inducing Protocols and Targeted Brain Stimulation

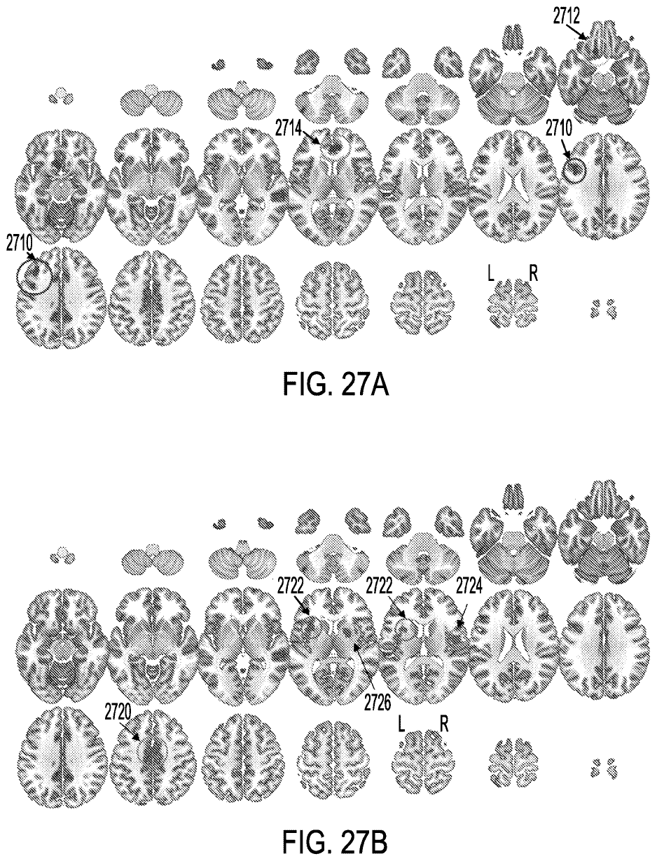

As described in the SYNAPSE section herein, some embodiments relate to systems and methods for modulation of brain plasticity. These methods include solutions to enhance overall brain plasticity via chemical agents (for instance, drugs) able to modulate the extracellular matrix and perineural net, promoting neuronal repair in the case of lesions and enhanced adaptability of neuronal systems, of relevance in neurological conditions such as traumatic brain injury and spinal cord injury, as well as in neurodegenerative disorders such as Alzheimer's Disease. These methods provide a generalized effect over lesioned central and peripheral neural tissue as well as over PNN of brain cells, with limited targetability of effects. The system and methods described herein allow for the combination of neuroplasticity-inducing approaches with non-invasive brain stimulation (NIBS) methods included in STIMOLA, for combined targeting using drugs and NIBS, targeted plasticity enhancement, pre-activation of neural pathways via NIBS to enhance the effect of drugs, and accelerated recovery from injury. Examples of applications for the synergistic effect of drug agents and STIMOLA are displayed in FIGS. 27A-27E, involving the application of both central and peripheral electrical and magnetic stimulation.

In some embodiments, noninvasive brain stimulation may be delivered over brain regions controlling peripheral systems of the body affected by a traumatic lesion or neurodegeneration. Stimulation may be performed to induce a synergistic effect with that of drugs acting on neuroplasticity and the PNN (SYNAPSE). Applications include rehabilitation protocols in patients with a form of spinal cord injury, traumatic brain injury, or a neurodegenerative disorder including but not limited to dementia, Alzheimer's Disease, Mild Cognitive Impairment, Frontotemporal Dementia, Multiple Sclerosis, and Parkinson's Disease. In some embodiments, noninvasive brain stimulation may be delivered in combination with peripheral nerve stimulation to create timing-dependent effects over lesioned spinal cord pathways located at different heights within the spinal cord. Simultaneous central (brain) and peripheral (for instance, right hand) stimulation may be used to promote bottom-up and top-down re-afferentation via increased neuroplasticity promoted by drugs acting on PNN and neuroplasticity. In some embodiments, noninvasive brain stimulation may be delivered over brain regions affected by neurodegeneration or tissue lesioning (for instance, due to Stroke, vascular pathology, and/or traumatic brain injury). The effect may be synergistic with that of drugs affecting the PNN and neuroplasticity, which induce a generalized effect over the entire brain. The combined effect may maximize therapeutic effects over compromised regions, limiting the effect over healthy brain tissue. In some embodiments, noninvasive brain and peripheral stimulation may be delivered before a drug treatment acting on PNN and neuroplasticity, with the goal to pre-activate target neural pathways, increase local metabolic activity and activate plasticity mechanisms in preparation for the additive effect of drug agents.

In some embodiments, noninvasive brain and peripheral stimulation may be delivered concurrently to a drug treatment acting on PNN and neuroplasticity, with the goal to induce a synergistic effect over target neural pathways. In some embodiments, noninvasive brain and peripheral stimulation may be delivered after a drug treatment acting on PNN and neuroplasticity, as a safe approach to maintain the therapeutic effects while limiting the potential side effects of the drug treatment.

In some embodiments, noninvasive brain stimulation may be performed by using plasticity-inducing protocols including, but not limited to, theta-burst TMS, repetitive TMS, cortico-cortical paired associative stimulation, and paired pulse TMS.

In some embodiments, noninvasive brain stimulation may be performed by using plasticity-inducing protocols based on transcranial electrical stimulation, including but not limited to stimulation using oscillatory electrical fields with embedded high-frequency electrical stimulation, pulsed random noise stimulation, and pulsed burst of high-frequency energy within frequency bands relevant for activation of PNN and neural circuitry.

In some embodiments, noninvasive brain stimulation may be performed by using tCS or TMS to activate multiple brain regions on a specific sequence to induce a patterned brain activation supporting brain recovery and modulation of specific brain connections supporting brain plasticity processes.

In some embodiments, noninvasive brain stimulation may be performed by using tCS or TMS to activate one or multiple brain regions concurrently to receiving a drug acting on the PNN and neuroplasticity. The combined intervention may accelerate adaptation to external and internal stimuli presented during a learning protocol, maximizing learning rates and enhancement of cognitive function. Drugs acting on the PNN and neuroplasticity combined with tCS or TMS may be used to enhance cognitive functions such as, but not limited to, memory, language, perception, visuomotor coordination, and attention and executive functions. A similar combined approach may be used to enhance performance in specific tasks including, but not limited to, learning how to play a musical instrument, learning how to play a videogame, or academic learning.

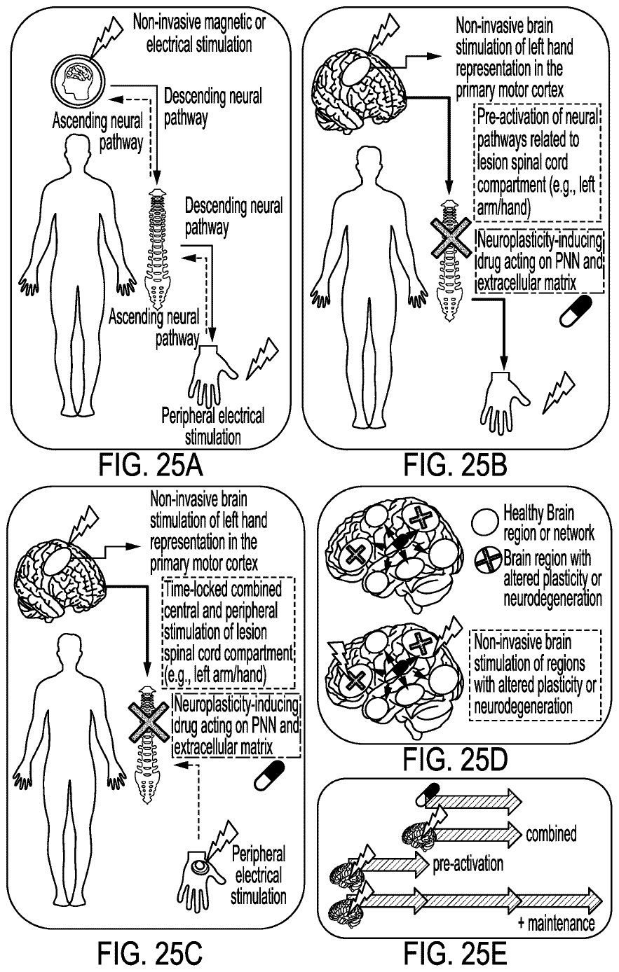

FIGS. 25A-25E show clinical applications of combined neuroplasticity protocols and noninvasive brain stimulation. FIG. 25A shows central (brain) and peripheral stimulation combined with an agent (for instance, a drug) acting on neuroplasticity via, for instance, modulation of the perineural net and extracellular matrix. FIG. 25B shows that in patients with spinal cord injury, noninvasive brain stimulation of the primary motor cortex or somatosensory cortex may be used to activate descending volleys reaching injured tracts of the spinal cord. Specific subregions of the motor cortex for maximal spinal cord engagement may be identified via PRINT and DARWIN algorithms and data. Stimulation may lead to activation of spinal cord tracts also affected by drugs affecting PNN and neuroplasticity, leading to a combined effect via synchronous endogenous (brain→spinal cord) and exogeneous (drug→spinal cord) stimulation. FIG. 25C shows that simultaneous brain and peripheral stimulation may be used to elicit a top-down and bottom-up effect over injured spinal cord tissue, promoting regeneration. Exact timing of brain and peripheral stimulation may be computed via PRINT and DARWIN to maximize simultaneous engagement of lesioned tissue. The effect may be synergistic to that of drugs affecting PNN and neuroplasticity locally. FIG. 25D shows that the system and methods described herein may be used in pathologies affecting the brain alone, where drugs affecting PNN and neuroplasticity exert a generalized effect over the entire brain. Noninvasive brain stimulation may be used to target brain regions or networks with altered plasticity levels or neurodegeneration, amplifying the effect of drugs. FIG. 25E shows that synergistic effects can be achieved via concurrent application of drugs and noninvasive stimulation by using brain stimulation to pre-activate damaged pathways or brain tissue, as well as by using brain stimulation to maintain the effect of drugs after a drug treatment.

### Induction of Cortical Brain Plasticity via Noninvasive Brain Stimulation in Patients With Alzheimer's Disease

Patients with mild-to-moderate Alzheimer's Disease (AD) underwent a series of assessments to quantify levels of brain cortical plasticity before and after a brain stimulation intervention aimed at increasing brain plasticity. The brain stimulation intervention, based on tACS targeting regions affected by altered plasticity mechanisms in the AD brain, was delivered over multiple days using personalized stimulation parameters based on DMDT information and optimized via DARWIN. Patients' data used for optimization included structural and functional brain scans, cognitive performance data and electroencephalography (EEG) data. tACS was delivered via Ag/AgCl electrodes placed on the scalp according to the international 10/20 EEG system.

Brain cortical plasticity was measured by applying a magnetic perturbation to the brain using TMS while recording brain cortico-spinal excitability levels longitudinally using electromyography (EMG). Magnetic perturbation was based on a high-frequency protocol configured to induce changes in cortical plasticity in the healthy brain. Specifically, a protocol known to increase cortical excitability was used, allowing measurements of the amount of change in excitability induced by TMS for a period of about 60 minutes after TMS delivery. In the healthy brain, high-frequency TMS induces an increase of excitability visible immediately after TMS delivery and extending for up to 30 minutes. The magnitude of excitability modulation is used as a measure of brain's response to perturbation and therefore as a measure of cortical plasticity (e.g., adaptability). Specifically, the size of motor evoked potentials (MEPs) recorded from the brain region stimulated via TMS is used to quantify brain excitability. Patients with AD report altered plasticity mechanisms and therefore display abnormal brain response to high-frequency TMS. Additionally, blood levels of Brain-Derived Neurotrophic Factor (BDNF) were measured before and after the tACS intervention, as a direct measure of the amount of expressed and circulating neurotrophic factors in the brain. BDNF is considered a gold-standard for the measurement of brain-wide brain plasticity levels.

To modulate brain plasticity mechanisms in the brain of AD patients, an intervention based on repeated tACS sessions was tested. tACS was delivered at a stimulation frequency known to interact with cortical mechanisms of plasticity in the healthy brain, therefore inducing an enhancement of plasticity levels in the brain. Brain plasticity levels were measured before and after the tACS intervention using the high-frequency TMS perturbation protocol described above.

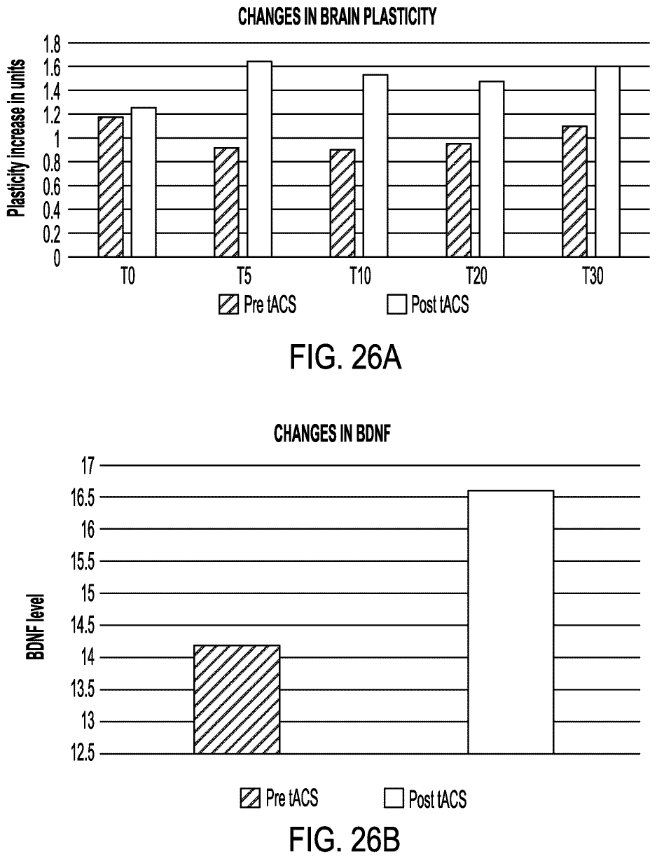

FIG. 26A shows that patients' brain excitability levels show a significant change after the tACS intervention (p.<0.05). Levels of plasticity were measured at multiple time points after tACS, showing a significant long-lasting increase of brain plasticity in AD patients in response to the personalized tACS intervention based on DMDT and DARWIN. Moreover, a significant increase in blood BDNF levels was also observed (p<0.018), suggesting a more generalized increase in brain plasticity due to increased production and circulation of neurotrophic factors, as shown in FIG. 26B.

FIGS. 26A-26B show enhancement of brain plasticity via brain stimulation in accordance with some embodiments. FIG. 26A shows changes in brain plasticity levels as measured via EMG data, recorded before and after the tACS intervention. Tx (e.g., T0, T5, etc.) represent the different timepoints (minutes) when plasticity levels were collected after TMS, showing a significant increase in plasticity extending for the entire measurement period. Pre and post tACS data points were collected over a period of at least 10 weeks, demonstrating long-lasting effects of tACS on brain plasticity levels in AD patients. FIG. 26B shows BDNF levels measured via blood samples before and after the tACS intervention. The BDNF levels also displayed a significant increase in brain plasticity levels after the intervention.

In this example, it was shown that optimized tACS stimulation based on individual DMDT data increases brain plasticity levels in patients with Alzheimer's disease. Normalization or enhancement of mechanisms of plasticity represents a key factor to counteract brain pathology related to Alzheimer's disease and dementia more in general. In some embodiments, interventions solely based on tACS for plasticity enhancement, as well as combined therapies using tACS in combination with drugs acting on the PNN and extracellular matrix, may be used to treat Alzheimer's disease and related dementia, including preclinical stages of the disease.

### Neuroscience of Learning and Change [IMPROVE]

In some embodiments, the systems and methods described herein may be used to accelerate learning, promote cognitive enhancement as well as psychological and behavioral change. BRAINPRINT and DARWIN may be used in conjunction with conceptual knowledge on cognitive neuroscience and neurophysiology to create a platform for accelerated learning. In this context, various brain modulators including, but not limited to, brain stimulation and cognitive-behavioral interventions, may be used to “prepare” the brain to learn, leveraging on a theoretical and analytical framework guiding the (i) selection and prioritization of cognitive functions and abilities to be strengthen to favor learning (e.g., sustain attention, Inhibition, cognitive flexibility), as well as (ii) brain properties associated with improved learning (e.g., brain plasticity level, corticospinal excitability). The system and methods described herein may allow to modulate and boost learning via multiple solutions, examples of which are described in more detail below.

In some embodiments, BRAINPRINT, DARWIN and STIMOLA may be used to directly facilitate, amplify, modulate, enhance, and trigger learning processes in the brain.

In some embodiments, BRAINPRINT, DARWIN and STIMOLA may be used to boost brain plasticity including, but not limited to, spike-timing dependent plasticity (STDP), Long-term Potentiation (LTP) and Depression (LTD), local plasticity circuitry at micro (e.g., cellular level) and meso scale (e.g., cortical columns level), and whole brain plasticity circuitry at the macro scale (e.g., structural and functional networks).

In some embodiments, BRAINPRINT, DARWIN and STIMOLA may be used to modulate global properties of the brain as a proxy of a general ability to learn, respond to perturbation, and adapt. Targeted mechanisms may include, but are not limited to, cortico-spinal excitability level, visual thresholds (e.g., phosphene threshold, circuitry supporting contrast sensitivity); auditory thresholds (e.g., auditory entrainment rate, auditory acuity); motor responsivity (e.g., motor reaction times, tactile sensitivity); Intra-cortical inhibition and facilitation (e.g., as measured via paired-pulse TMS), electrical or magnetic entrainment (e.g., as measured via combined electrical stimulation and EEG recording).

In some embodiments, BRAINPRINT, DARWIN and STIMOLA may be used to rewire certain specific connections in the brain important and/or crucial for learning. Targeted modulation may include, but is not limited to, protocols to change the synchronization between cognitive and sensory brain networks; protocols to increase synchronization within a given brain network to increase the rate of information processing; protocols to decrease synchronization between two brain networks to increase functional segregation; and protocols to synchronize two or more nodes/regions of a brain network in a specific order, following activation patterns and their specific phase information mimicking what observed during successful learning.

In some embodiments, BRAINPRINT, DARWIN and STIMOLA may be used to derive a series of brain modification steps mimicking processes and dynamics occurring during learning in humans. BRAINPRINT and DARWIN may suggest optimal targets to ignite, accelerate or prolong the effect of a learning process, such as in the case of learning a motor sequence (e.g., music training) or language processing (e.g., language acquisition). BRAINPRINT and DARWIN may use information from an individual performing a task and compare it with that of a proficient individual (“template-matching”) or that of a similar individual at progressively more difficult stages of learning, thus defining an optimal sequence of brain states maximizing the chance for learning. BRAINPRINT and DARWIN may also identify brain targets (e.g., regions, connections, networks) related to learning processes in general (e.g., related to an attention network), suggesting protocols for optimization of learning as a general state of the brain.

In some embodiments, a “Optimal Learning State” (OLS) may be promoted first via interventions modulating brain structure and function including, but not limited to, brain plasticity, cortical excitability and brain connectivity, followed by task-specific modulations introduced to specifically engage learning processes related to the desired state/trait (e.g., motor sequence learning). The OLS may be defined as a high plasticity, optimal flexibility, high network segregation, high motivation, high sensory activation, heightened inhibition, and/or higher resilience state.

In some embodiments, a specific sequence/hierarchy of changes/interventions may be identified for each individual brain based on BRAINPRINT and DARWIN, and the most appropriate interventions may be identified on the basis of knowledge about the magnitude, duration and dose-response effects of an array of available brain modifiers. For instance, an individual interested in improving visuomotor abilities related to hand-eye coordination (e.g., a racecar driver) whose BRAINPRINT suggests a (i) reduction of synchronicity in the visuo-motor system, an (ii) overall suboptimal level of brain plasticity and (iii) increased attention to external stimuli accompanied by low attentional levels, may be suggested a training course centered on first enhancing overall brain plasticity via non-invasive brain stimulation (to facilitate any type of follow-up training, and boost consolidation of knowledge), followed by a patterned oscillatory brain stimulation intervention targeting the visual and motor cortices (to increase synchronicity between the visual and motor systems), both completed in parallel to a mindfulness training course to increase separation between interoceptive and external stimuli, reducing information noise during execution of visuo-motor tasks.

In some embodiments, BRAINPRINT and DARWIN may be used to derive optimal brain targets for enhancement of specific cognitive and behavioral functions. Targets may include multiple brain regions or entire brain networks, whose activity may be modulated in order to reach a state of high performance. Based on knowledge derived from existing BRAINPRINT and DARWIN analysis, a portfolio of brain targets may exist.

In some embodiments, BRAINPRINT and DARWIN may be used to derive optimal brain targets to reach a state of high cognitive performance identified as the most common brain sites for fluid intelligence-related processing in the human brain. Brain state and trait analysis may be used to identify optimal sites for neuromodulation with the goal to, but not limited to, enhance overall global cognition related to fluid intelligence and executive functions as well as boost learning.

In some embodiments, BRAINPRINT and DARWIN may be used to derive optimal brain targets to reach a high cognitive performance brain configuration identified as the convergence of convergent and divergent thinking in humans. Systems/regions/networks supporting the high cognitive performance state may include, but are not limited to, those related to insight abilities, fluid intelligence, executive functions and creativity. Resulting systems/regions/networks may be constituted by a multilayer network, including brain nodes common to each function as well as those only partially overlapping across domains.

In some embodiments, the systems and methods described herein may use a library of treatment-response data (e.g., effect of a specific memory training on the hippocampus or prefrontal cortex), and then optimize solutions to maximize their impact on those specific brain regions according to any given condition of interest. In this way games and/or training apps may be specifically designed to, e.g., increase blood flow in regions crucial for memory or language processing, or increase the synchrony between brain networks responsible for sustained attention or decision making.

In some embodiments, a device for brain monitoring and brain modulation (e.g., PERCEPTRON) may be used to, at least in part, track cognitive status and proficiency at given tasks via EEG, deploy cognitive training strategies to strengthen cognitive functions, and use brain stimulation at baseline to prime the brain (e.g., increase plasticity) or reinforce learning by stimulating during the execution of a target cognitive task. Users may receive a personalized recipe based on baseline assessments performed with the device, and including a set of personalized steps to approach any learning experience.

In some embodiments, a quantitative map representing brain areas and networks whose patterns of activation are responsible for the generation of a placebo response, may be used as targets to accelerate learning and brain change. Placebo effects have been shown to correlate with increased learning rate and positive cognitive as well as behavioral outcomes, by driving a shift in brain dynamics. The systems and methods described herein include knowledge on the number, location and role of specific brain regions involved in placebo response, as well as their correspondence with brain functional networks and the pattern of activation and deactivation needed to elicit a placebo response. These patterns may be used to identify areas to be modulated before, during and/or after e.g., a given learning paradigm, therapy session, or cognitive enhancement procedure. Procedures for obtaining details on brain targets, networks and specific brain dynamics to be modulated are described in more detail below.

In some embodiments, knowledge on the most relevant brain systems/regions/networks and cognitive functions relevant for learning, plasticity and brain change may be used to guide prescription and administration of verbal therapy including, but not limited to, psychotherapy, cognitive-behavioral therapy (CBT), exposure therapy, dynamic therapy, cognitive restructuring, psychological counseling, trauma-focused therapy. BRAINPRINT and DARWIN may be used to identify optimal targets for neuromodulation aimed at increasing therapeutic efficacy, accelerate therapeutic efficacy, decrease attrition, and/or prolong clinical benefits.

In some embodiments, IMPROVE may be used to identify optimal brain targets for priming, enhancement and maintenance of psychotherapy effects on wellbeing and mental health. Integration of precision fingerprinting of patients via BRAINPRINT and DARWIN with neuromodulatory interventions from STIMOLA directly targeting disease-specific brain targets related to brain plasticity may lead to cumulative or exponential clinical effects.

In some embodiments, IMPROVE may be used to define optimal, personalized hierarchies of interventions to be deployed in patients with psychological or psychiatric conditions, based on information from an individual's DMDT and cognitive optimization algorithms from DARWIN.

### EXAMPLES

Some embodiments described herein are further illustrated by the following examples and detailed protocols. However, the examples are merely intended to illustrate embodiments and are not to be construed to limit the scope of the technology described herein.

### Placebo Response and its Effect on Motivation, Performance and Clinical Symptoms

In some embodiments, the systems and methods described herein may allow to identify and target specific brain regions and networks linked to a placebo response, given the documented link between placebo response and effectiveness of therapeutic interventions aimed at changing brain properties, cognition, behavior and clinical symptoms of medical conditions, as well as the impact of placebo on learning and plasticity.

The complex neurobiology of placebo effects may underlie a more complex interplay of large-scale brain networks. Research in different fields has shifted from the investigation of functional properties of isolated brain regions, to their connections, in line with the emerging interest in large-scale functional cortical networks dynamics. Brain regions are linked by structural (anatomical) or functional (dynamic) connections able to engage together in different and complex patterns. The synchronized activity between spatially distinct interconnected areas within so-called resting-state functional networks (RSNs) can be measured via functional connectivity (FC) analysis using functional MRI data. RSNs include regions responsible for both sensory processing and high order cognition, working together to form specific networks. Knowledge on the brain networks involved in a placebo response may be applied towards the enhancement or inhibition of the placebo response as a therapeutic intervention, with relevance for a variety of neuropsychiatric disorders as well as cognitive enhancement and brain health.

Some embodiments of the system and methods described herein include knowledge on the localization of brain networks supporting a placebo response, as well as their specific interplay required to elicit a placebo response of cognitive and behavioral value. A description of placebo-relevant brain structures and networks and potential brain targets for placebo-induction obtained via DARWIN is provided below. Targets may be modulated via methods including, but not limited to, noninvasive brain stimulation.

### Placebo Mapping

A seed-based connectivity analysis examining the spatial similarity of voxel-wise connectivity maps may be conducted to identify regions showing activation during a placebo response. The average BOLD fMRI time course during resting-state may be retrieved by averaging the signal from all the voxels included in a resting-state map. Subsequently, the signal from each map may be correlated with that of the remaining voxels in the rest of the brain, resulting in a 3D volume where each voxel value represents the correlation coefficient between its BOLD activity and that of the seed map of interest. Results may be computed applying a voxel-level threshold and cluster size correction.

The spatial similarity of seed-based connectivity maps may be calculated using a similarity metric to investigate the overlap with functional brain networks including, but not limited to, the visual network (VS), the ventral and dorsal attention (DAN and VAN), the somatosensory (SM), the limbic (LIM), the default mode (DMN), and the fronto-parietal control network (FPCN).

The comparison of weighted, un-thresholded connectivity maps for activation/deactivation clusters in the placebo effect and RSN map at the single voxel level may require considering not only spatial similarity but also the similarity of connectivity sign (i.e., positive and negative connectivity). A similarity index may be obtained by computing the product of each voxel's value across two maps (e.g., voxel j in placebo maps and DMN maps), resulting in a map where positive values represent voxels with the same sign in both maps (e.g., positive connectivity in both placebo and DMN), while negative values represent opposite signs (e.g., positive connectivity value in voxel j in placebo, negative in DMN). As a result, the magnitude of the similarity index may represent the similarity of connectivity strength in any two given maps.

FIGS. 27A-27B show clusters of brain activation (FIG. 27A) and deactivation (FIG. 27B) in anticipation and/or during placebo response. Three primary brain regions 2710, 2712, 2714 whose functional activation is relevant for placebo response were identified, as shown in FIG. 27A. Four clusters of brain regions 2720, 2722, 2724, 2726 whose functional deactivation is relevant for placebo response were also identified, as shown in FIG. 27B.

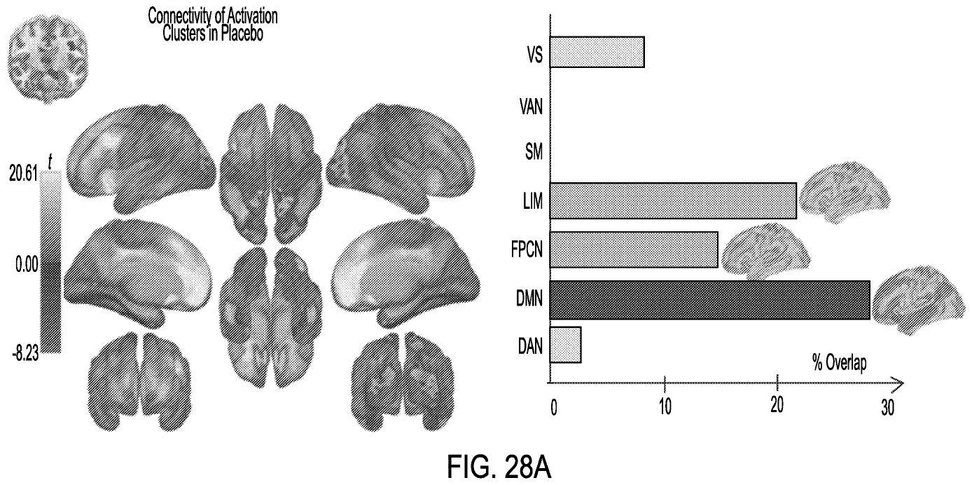

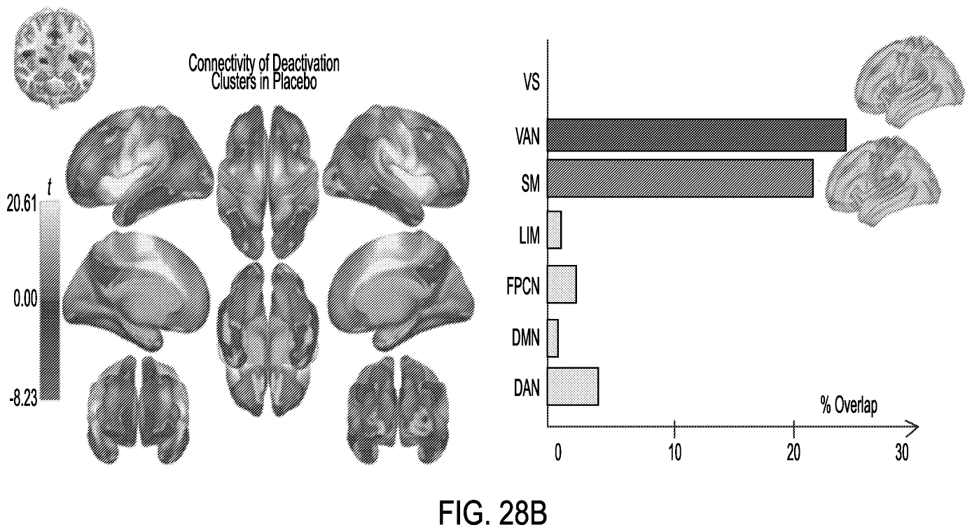

FIGS. 28A-28B show network mapping of placebo clusters. Similarity coefficient for connectivity maps of activation (FIG. 28A) and deactivation (FIG. 28B) clusters. VS: visual network. VAN: ventral attention network. SM: somatosensory network. LIM: limbic network. FPCN: fronto-parietal control network. DAN: dorsal attention network. DMN: default mode network. A quantitative similarity analysis was used, showing strong similarity between placebo-associated brain activation clusters and the default mode (DMN), the fronto-parietal control (FPCN), and the limbic (LIM) networks; a complementary pattern across the deactivation clusters was obtained, with higher similarity for VAN and SM networks, as shown in FIGS. 28A-28B.

DARWIN analysis suggests that the optimal placebo response may be achieved via simultaneous activation of systems/regions/networks related to high-order cognition, executive functioning, internal dialogue, motivation and expectancy, and deactivation of sensory regions together with decreased attention towards incoming external stimuli (e.g., decreased vigilance).

In some embodiments, brain neuromodulators including, but not limited to, noninvasive brain stimulation approaches described in STIMOLA and behavioral interventions, can be used to directly elicit a brain activation or deactivation in systems/regions/networks identified via DARWIN, thus inducing a placebo response which may be used to, at least in part, accelerate learning, enhance, boost cognition, enhance the effect of pharmaceutical interventions as well as behavioral and cognitive interventions, promote psychological and behavioral change.

In some embodiments, systems and methods described herein can be used to pre-activate brain regions related to placebo response before the execution of a mental, cognitive or physical activity. Pre-activation can be obtained, for instance, via behavioral exercises triggering a metabolic response in placebo-related brain regions, and via direct noninvasive brain stimulation of said regions.

### Clinical Applications of Placebo Mapping and Induction

In some embodiments, the brain network supporting placebo responses in humans may be used to quantify the level of clinical, cognitive or brain response to a given treatment by measuring the level of network activation. The approach may be used to estimate individual responsiveness to placebo induction, which may be then used to define dose-response studies to identify personalized dosing of a given treatment while accounting for an estimated placebo response.

In some embodiments, the identified brain network supporting placebo responses in humans may be used to quantify the level of clinical, cognitive or brain response to a given treatment by measuring the level of network activation. The approach may then be used to evaluate the impact of a hybrid treatment protocol where a combination of real and placebo substances is administered for the treatment of a given medical condition.

In embodiments, real and placebo pills are administered in a random or predefined sequence over the course of a treatment period, and both clinical, cognitive and brain responses are measured in response to different ratios of real/placebo pills. Placebo pills may be composed so that they replicate the same features of a corresponding real medication, including but not limited to organic structure, taste, smell, and texture. In an example scenario, patients are administered with a ratio of 80/30 real/placebo pills, with a measured efficacy over clinical symptoms of 80%; in a subsequent study, 70/30 ratio is used, and clinical efficacy may be measured at 76%, as well as activity in the placebo brain network; the same process may be followed until no clinical benefits are obtained. A cost-benefit analysis considering clinical effects as well as side/adverse events may be conducted, determining the optimal ratio of real and placebo pills to be administered to obtain the highest clinical impact with the lowest rate of side/adverse events. Hybrid placebo treatments may be used to decrease habituation to drugs while maintaining clinical benefits, as well as to decrease side and adverse effects.

### Modular, Adaptive, Neuro-Approach for Personalized Virtual and In-Person Psychological Therapy and Empowerment

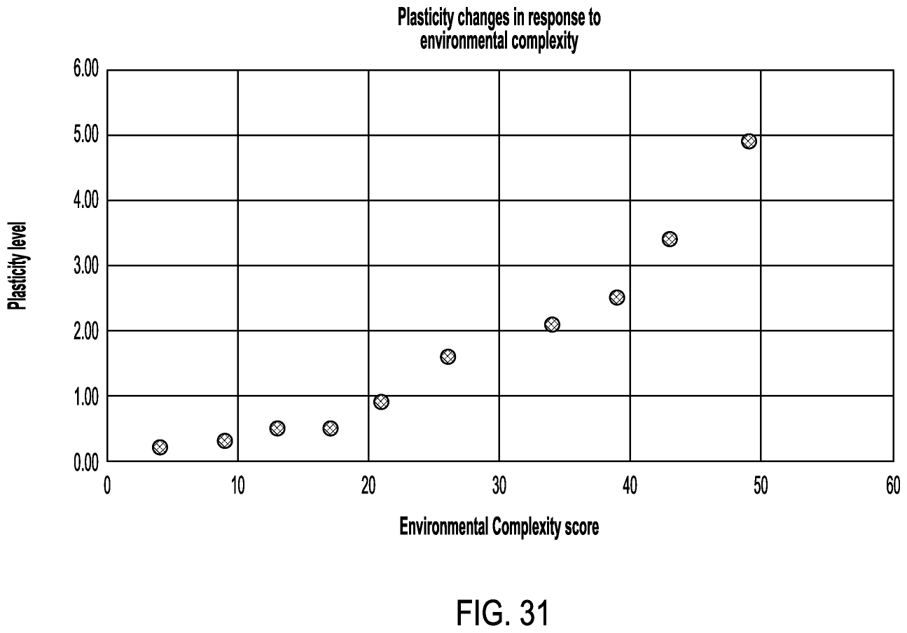

Algorithms from DARWIN may be applied to IMPROVE to generate personalized trajectories for cognitive and psychological change used in the context of psychotherapy and clinical psychology interventions. Individual trajectories for psychological change are composed by modules addressing cognitive and psychological constructs at the core of human psychology as defined by the principles of cognitive-behavioral psychotherapy, as well as ad-hoc modules derived from an individual's DMDT data and DARWIN analysis. The Modular, Adaptive, Neurological approach to Psychological Change (MANP) includes an original analysis of basic human psychological and cognitive abilities necessary for psychological change, as shown in FIG. 31, including brain regions, systems and networks related to, but not limited to: Insight abilities, long-term memory, visual imagery, meta-cognition, procedural planning, cognitive flexibility, metaphor production, empathy, locus of control, emotion processing, control of autonomic response. These functions are defined as standalone construct with related abilities for an individual to master to either (i) gain insight on a psychological issue or (ii) promote change and resolution of psychological symptoms.

The MANP is based on two levels of inference: a (i) psychological one, based on existing and novel notions related to psychological constructs and abilities, and a (ii) biological one, related to the localization and co-localization of said psychological constructs and abilities; the 2-layer approach allows to identify a hierarchy of psychological abilities based on their localization in the brain and corresponding likely response to perturbation/stimulation; for instance, given a psychological condition where psychological change requires acknowledgement of emotional distress and a past trauma, may require acquisition of skills and abilities related to, but not limited to, visual imagery (for visualization of past traumas), insight (for conceptualization of the nature of present stress and possible causes), inhibition (for suppression of excessive irrational reactions to past traumas and memories), long-term memory (for recollection of past events), metaphor production (for verbalization and generalization of novel insight and knowledge on present condition), an external locus of control (to externalize the sense of responsibility for past traumas). The order in which these abilities are acquired or developed may be important to the success of a psychological treatment, and such order should consider the (i) existing hierarchy of psychological and cognitive skills of a given patient, and (ii) the hierarchy of activation and dependency of said skills in terms of their physical and functional representation in the brain. For instance, abilities related to language (e.g., internal dialogue, insight, metaphor production) are represented in the temporal lobes of the brain, and are hierarchically controlled by higher-order cognitive constructs related to executive functions such as planning, inhibition, flexibility and working memory. An intervention prioritizing the acquisition of language-based abilities without a prior training of executive functions may lead to delayed or null results. Selection of the right hierarchy and sequence of interventions for any given patient may be helpful to ensure maximal psychological benefit in the shortest amount of time, significantly shortening months or years of therapy.

Psychotherapy is mostly conducted via verbal therapy and sporadic use of activities aimed at facilitating psychological insight via practical exercises or interactive activities. However, many abilities addressed in a psychotherapy context involves a level of sensorial stimulation and interaction not achievable via verbal communication. For instance, visualization of a traumatic memory, treatment of a specific phobia, experiential social interaction, locus of control exercises, or visual imagery exercises, may be more effective when conducted in real-life settings or by means of VR/AR/XR technology. In some embodiments, MANP includes hybrid solutions based on VR/AR/XR technology, according to the hierarchy of abilities and psychological constructs. Ad-hoc VR/AR/XR applications may be created for two main objectives: (i) to accelerate psychological change via a more immersive delivery of techniques addressing main psychological constructs and abilities (for instance, an ad-hoc VR app for rehearsal of a social situation causing stress), and (ii) to facilitate brain changes via sensory and cognitive stimulation apps not related to any specific construct or ability but instead aimed at changing brain activity in specific brain network and systems at the core of psychological change (for instance, visual and auditory 3D sensory stimulation to create an enriched environment inducing a state of elevated brain plasticity, with consequent higher likelihood of brain and cognitive rewiring; see SYNAPSE for information on enriched environment's effect on brain plasticity).

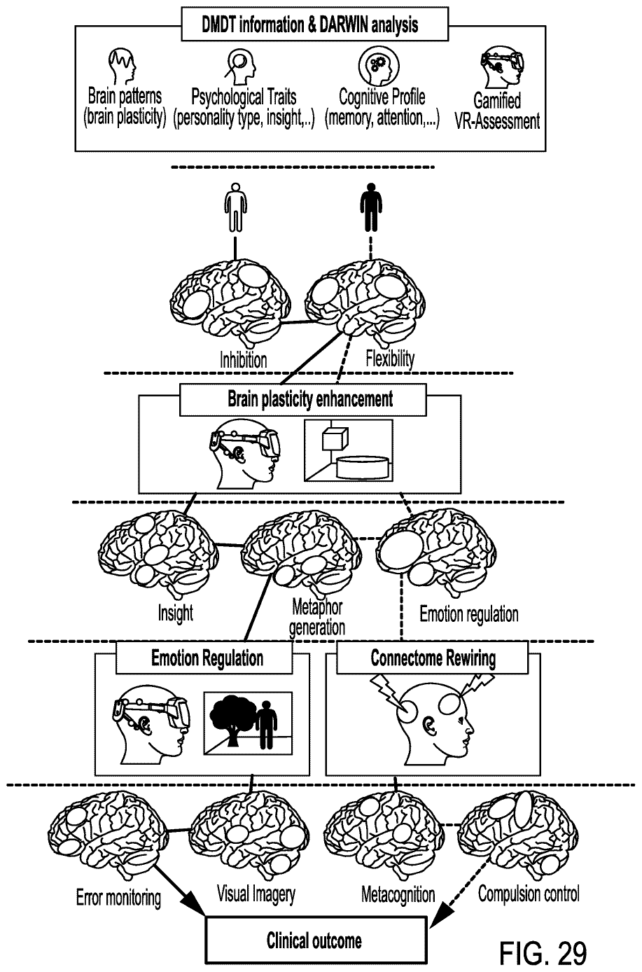

As shown in FIG. 29, MANP includes a combination of verbal and augmented therapy, where modules and techniques targeting both specific psychological abilities and general properties of the brain compose a unique, personalized therapeutic trajectory for each patient.

FIG. 29 shows a MANP hierarchical approach in accordance with some embodiments. Examples of two personalized therapeutic trajectories defined based on DMDT data from two patients and analysis using DARWIN algorithms are shown. Based on DMDT and DARWIN estimates, two sequences of hierarchical psychological abilities may be defined for each patient, maximizing psychological change and therapy effectiveness. The interventions also include applications targeting brain physiological processes and mechanisms known to facilitate brain and cognitive change, e.g., brain plasticity. In some embodiments, modulation of plasticity may be achieved via a VR application combining a complex scenario with sensory stimulation to produce an enriched environment. Progressing through the hierarchy, VR applications may also be used, for instance, to enhance emotion regulation abilities in a patient, whereas noninvasive brain stimulation may be used in a different patient to change patterns of brain connectivity otherwise impeding change and adaptation in specific brain network supporting psychological functions. As a result, each patient may be exposed to a personalized, adaptive, modular intervention involving multiple media and techniques as part of the systems and methods described herein (e.g., BRAINPRINT for patients' initial assessment, DARWIN to define personalized clinical trajectories, SYNAPSE for plasticity modulation, STIMOLA for connectome rewiring).

In some embodiments, MANP is used in combination with digital psychology tools including, but not limited to, neurofeedback and biofeedback, for the online monitoring of physiological, cognitive, psychological and neural state either before, during and/or after the delivery of a specific intervention. Neurofeedback and biofeedback may be delivered via PERCEPTRON. In some embodiments, MANP may include a platform for digital learning called REMOTELY where providers and users can interact, share resources and information, including delivering remote psychotherapy and counseling sessions.

In some embodiments, MANP may be used in combination with VR applications displayed via headsets and/or visors such as PERCEPTRON or displayed directly on a web-browser to facilitate remote delivery of therapy and adherence.

In some embodiments, MANP may include interventions for the treatment of conditions related to sleep including, but not limited to, sleep disorders such as insomnia, sleep apnea, REM-sleep disorder and patterns of sleep alterations linked to other conditions such as major depression, anxiety disorders and physiological/pathological aging. Some applications include the combination of augmented and virtual reality for the delivery of gamified therapeutic applications where light stimulation is embedded in the game or app engine. Light stimulation may be performed with different intensities according to the time of the day and circadian rhythms personalized to an individual's habit and sleep patterns. Stimulation may be performed within a spectrum range between >200 and <1250 nm, personalized for each individual receiving the treatment. Aspects of the application and/or game may be composed by educational material related to sleep disorders and sleep hygiene, others may be presented in the form of a serious game or videogame where the light stimulation component is not central to the experience but embedded in the background, others may constitute modules of a cognitive behavioral therapy for the treatment of sleep disorders. Personalization of the psychotherapy, gaming and light stimulation may be based on DMDT and DARWIN algorithms.

In some embodiments, combined light stimulation, psychotherapy and gamified interventions may be used to modulate sleep patterns and exert an effect on the activity of the glymphatic system of the brain and cerebrospinal fluid (CSF) circulation in individuals with cognitive symptoms and a diagnosis of dementia. In some embodiments, the same (or similar) intervention may be applied to healthy, asymptomatic individuals as a preventative measure to delay the onset of neurological disorders, in particular Alzheimer's disease and related dementias, mild cognitive impairment and frontotemporal dementia.

### Conversational A.I. Agent to Augment Clinical Effects Through Brain Plasticity Induction

Progress made during a psychotherapy session or similar mental health intervention is often limited by their acute effects and lack of consolidation once a therapy session is over. Neurobiological processes related to brain plasticity, memory consolidation and programmatic learning may be used in some embodiments to consolidate knowledge and insight acquired while receiving a therapy. MANP includes a platform for Offline Consolidation through Plasticity (OCP) where an A.I. algorithm is used to identify the best additional intervention to be deployed in-between therapy sessions. Interventions may be based on the concepts of brain plasticity (e.g., SYNAPSE) and learning (e.g., IMPROVE) and are aimed at maximizing clinical efficacy. Cognitive, behavioral, psychological and biometric data collected in DMDT and updated during the application of MANP may be used to identify key topics and psychological constructs requiring consolidation of knowledge and recent insight achieved via therapy.

In some embodiments, an A.I. powered OCP conversational agent may be used to guide an interactive platform for patients to evoke and elaborate on cognitive and psychological concepts and topics relevant for the therapeutic trajectory identified for each patient via MANP. The concepts and topics may have been addressed during the last therapy session or constitute preparatory work for upcoming sessions. The agent may be deployed via an app available on multiple media including, but not limited to, mobile phones. The OCP conversation agent may be based on principles from the NEURO-AI module, with the selection of topic and concepts to be addressed being guided by individual MANP trajectories and by learning-to-learn principles from IMPROVE. For instance, a progression algorithm may be defined where a concept or topic is labeled as “achieved” only when a certain proficiency is reached; proficiency or mastery of a psychological, cognitive or emotional state is determined via:
(i) analysis of verbal inquiries and corresponding answers, using proprietary algorithms for voice pattern analysis based at least in part on emotion, stress and deception recognition;
(ii) analysis self-report measures collected on patients via questionnaires coded and processed by a behavioral analysis algorithm;
(iii) analysis of biometric data collected via wearables and devices including but not limited to PERCEPTRON, including at least in part stress markers such as galvanic skin response and heart-rate variability, and brain signal data including at least in part EEG data and corresponding markers of oscillatory activity (including analysis of brain states described as part of Consciousness Sampling and Classification (CSC) in DARWIN);
(iv) analysis of facial patterns via a camera including but not limited to an optic or thermal camera, with automatic classification of emotional state (including but not limited to core emotions such as anger, sadness, happiness, worry), cognitive and psychological state (including but not limited to being stress, brain fog, cognitive exhaustion); and
(v) analysis of eye-movement via a dedicated eye-tracking apparatus configured to monitor eye movement patterns including saccades, gaze, micro-saccades, both during spontaneous mentation and in response to external sensory stimuli.

In some embodiments, an augmented environment may be used to promote brain plasticity via a VR-based 3D environment where A.I. is used to define physical properties of objects with the goal of creating increasingly complex cognitive/sensory stimulation patterns. Additional details on this approach are included in the Virtual Augmented Environment (VAE) application described in the NEUROCREATOR section herein.

In some embodiments, a form of noninvasive brain stimulation known to elicit brain plasticity may be used to prime and/or consolidate a brain region, circuit, network or system before or after a therapy session.

In some embodiments, a placebo response may be induced to elicit a positive psychological state with the goal to augment intrinsic motivation to change and consolidate previously acquired knowledge. Activation of the Psychological & Cognitive Change (PCC) network of the brain may be used in this context to enhance and amplify therapeutic effects.

### Conversational A.I. Agent for Cognitive and Functional Enhancement [CLARITY]

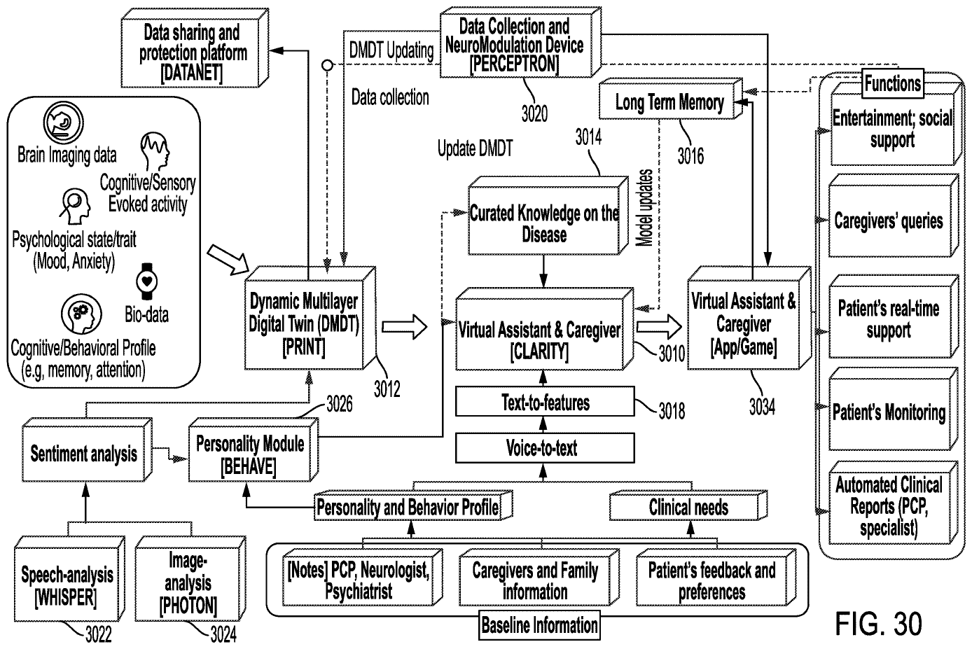

In some embodiments, a conversational A.I. agent may be used to create a virtual caregiver/assistant for patients with deficit of functional independence including, but not limited to, patients suffering from neurodegenerative diseases such as Alzheimer's Disease, Frontotemporal Dementia, Mild Cognitive Impairment, Amyotrophic Lateral Sclerosis, Subjective Memory Complain. The A.I. agent (referred to herein as “CLARITY”) can ingest information about an individual, both from text and voice including, but not limited to, notes from primary care physician, specialists (including, but not limited to, a neurologist, cardiologist), family members, friends and the patient themselves, to create a personalized digital twin of the patient's clinical, psychological, cognitive, behavioral and social ecosystem and data. The data may then be used to tune the personality and behavior of the agent, whose role is to monitor patient's activity through the day, send reminders of important activities, and/or interact with the patient for emotional and social support. FIG. 30 shows a schematic representation of an example software architecture for CLARITY 3010 in accordance with some embodiments.

In embodiments, the agent is composed by a central autonomous agent connected to a DMDT 3012 of the user; a module 3014 with curated knowledge on the specific medical condition affecting the user; a long term memory module 3016 to record and store every interaction between users-agent and update knowledge on the user; a feature extraction module 3018 to convert verbal and written inputs into features classes to inform the autonomous agent; an interface or multiple interfaces for multiple users; an interface to receive and data from a portable data acquisition device (e.g., PERCEPTRON 3020).

In some embodiments, the agent is configured to interpret voice messages and notes via a speech analysis module 3022 (WHISPER) performing sentiment analysis with relation to emotional state, level of engagement, activation level, stress levels.

In some embodiments, the speech analysis may include biomarkers of cognitive dysfunction and neurological symptoms including, but not limited to, tremor levels to investigate motor symptoms, words cadency, words frequency, grammar, unusual words.

In some embodiments, the speech analysis module may be used for automatic interpretation of an individual's notes recorded spontaneously during the day. Results may be included in the DMDT and may be used to generate reports for caregivers including, but not limited to, family members, primary care physicians and specialists such as neurologists and psychiatrists.

In some embodiments, the agent may be configured to interpret emotional states via a video-analysis module 3024 (PHOTON) analyzing images captured via a portable camera. The module may perform a sentiment analysis with relation to facial features including but not limited to level of engagement, activation level, attention levels, stress levels, and emotional state based on a set of emotion templates personalized to the user. Results may be included in the DMDT and may be used to generate reports for caregivers including, but not limited to, family members, primary care physicians and specialists such as neurologists and psychiatrists.

In some embodiments, a personality module 3026 (BEHAVE) is configured to receive inputs from PHOTON 3024 and WHISPER 3032 analysis modules and adjust the behavioral features of the agent in real-time. The baseline personality features of BEHAVE may be derived from information extracted from, but not limited to, a user's DMDT, user's preferences evaluated via an initial questionnaire, and/or caregivers' suggestions on most effective ways to interact with the user. BEHAVE 3026 may be configured to constantly update its behavior based on a user's interactions with the conversational A.I. agent stored and elaborated by the long-term memory module 3016.

In some embodiments, the agent may be deployed via a specific medium including, but not limited to, an avatar in a videogame application 3034 for cognitive training prescribed to the patient. In the game, the avatar function as a non-playable character may assist the patient in the completion of various tasks.

In some embodiments, the agent can be queried by family members about the status of patient's cognition, behavior and activities. In some embodiments, the agent can be queried by a primary care physician or specialist about the patient's daily activities, and can produce a report including cognitive and behavioral notes about the patient, including notes from caregivers. In some embodiments, the agent can be queried via a portable device including but not limited to a mobile phone. In some embodiments, the agent can be queried via web portal hosted on a specific domain, and is linked to a QR code that can be scanned with a portable camera for direct access to the portal.

In some embodiments, the agent may be used as a remote assistant for patients living in assisted living facilities, to manage their schedule, provide company, and provide cognitive engaging activities throughout the day.

In some embodiments, the agent may be used to assist patients receiving mental health support to maximize the efficacy of psychotherapy interventions. Information, therapist notes, and curated summaries of psychotherapy sessions are uploaded to the long-term memory module 3316, together with prescriptions by the therapist. The agent may remind the patient of tasks to be completed during the week based on assignments by the therapist, topics addressed during prior therapy sessions, and can collect notes from the patient in order to suggest where to resume the patient's internal dialogue when prompted.

In some embodiments, the agent may be equipped with long-term memory and a learning algorithm, may be configured to accumulate knowledge on the patient's diagnostic and therapeutic history, and may be configured to generate patterns of reasoning based on current psychotherapy models.

### Sleep Induction Interactive Platform

Brain oscillatory state before falling asleep is associated with better sleep quality including, but not limited to, longer time spent in deep sleep stages, less fragmented sleep and therefore improved CSF dynamics, protein clearance and better memory consolidation. Data suggests that a specific ratio of slow and high-frequency brain oscillation detected in a period between 30 minutes and 3 hours before falling asleep is predictive of sleep quality, and that engaging in specific cognitive, sensory, and psychological activities before going to sleep will improve sleep quality and behavioral/psychological/cognitive/neurological consequences. Data also suggests the location where slow and high frequency activity should happen in order to favor sleep induction and sleep maintenance during the night, with a particular focus on higher slow-frequency activity in the frontal lobes and suppression of attention-related oscillatory activity in the posterior part of the brain including the occipital cortex and the visual system.

In some embodiments, MANP may be used to identify cognitive tasks, behavioral, and sensory tasks able to generate the target pre-sleep brain state for a specific individual, combining data from, but not limited to, DMDT, DARWIN, SYNAPSE and IMPROVE. In addition to said data, an assessment of individual response to the selected tasks may be performed, with the individual being exposed to multiple cognitive, sensory and behavioral perturbations while their brain oscillatory activity is recorded via PERCEPTRON. Immediate response to perturbation, described as spectral power changes in slow and fast frequency bands, may be used to identify the most effective tasks in evoking the desired target pre-sleep state. After the initial assessment, a digital application may be created, and the individual may be prescribed to interact with the app before going to sleep.

In some embodiments, the application may be implemented as a videogame application played on a mobile device including, but not limited to, a smartphone, a tablet, or a VR device. In some embodiments, the videogame may be played while receiving concurrent cognitive and sensory stimulation, with sensory stimulation including, but not limited to, auditory stimulation and visual stimulation. Auditory stimulation may be embedded in the audio features of the videogame; the stimulation may include a frequency gradient, progressively transition the individual from high-frequency stimulation in the gamma band to slow-frequency stimulation in the delta range. The transition may be based on performance in the game, constantly adapted in real-time based on user's performance in terms of sustained attention, divided attention, and/or reaction times.

In some embodiments, visual stimulation may be delivered via manipulation of physical properties of in-game objects including, but not limited to, their shape, color, brightness, and/or position on the screen. Physical properties of the objects may be manipulated over time to evoke specific patterns of brain activity in slow and high frequency bands. The frequency of change over time may determine a rhythmic, patterned stimulation of the visual system which modulates activity in the visual cortex of the brain, inducing the target oscillatory activity linked to better sleep quality.

In some embodiments, sensory stimulation may be adapted based on brain oscillatory activity data collected from the user via a PERCEPTRON device placed on the head while playing the videogame application.

### EXAMPLE METAVERSE AND GAMING APPLICATIONS

Some systems and methods described herein have applications in the gaming industry and the metaverse. The metaverse is a theoretically infinite space where the physics of generated content and experiences offered to users are bound to the imagination of developers, designers and digital architects. The metaverse leverages technology and innovation from the fields of digital art and gaming for what concerns the creation of 3D virtual environments and social dynamics, while also leveraging behavioral information related to patterns of activity and digital footprint to generate personalized experiences for users and gamers. While behavioral analysis provides interesting material for the creation of unique gaming or VR experiences, direct access to brain activity using some of the methods and system described herein may allow to create a novel class of content, applications and platforms specifically tailored to an individual central nervous system, including its structural architecture as well as passive and evoked patterns of activity unique to each individual.

Some embodiments of the system and methods described herein include a NEUROCREATOR module configured to collect, analyze, characterize, and manipulate information related to the central nervous system of an individual, to generate or manipulate content in the metaverse and gaming applications, including VR, XR or AR experiences. Within NEUROCREATOR, modules such as BRAINPRINT, PREPARE and DARWIN may be central for the development of a “Neuroverse”, allowing to collect, analyze, predict and modify brain activity at the individual level. In an example scenario, BRAINPRINT may be used to inform, create, or derive digital avatars and digital contents based on DMDTs. In a different scenario, DARWIN may be used to evolve, improve, and/or modify digital avatars and digital contents. In another scenario, methods from STIMOLA may be used to enhance in-game abilities and performance, accelerate learning, improve and/or modify digital avatars by affecting users' brain activity, physiology, and cognition.

Described in more detail below are some embodiments of NEUROCREATOR, including example applications for the metaverse and for the gaming industry. Applications such as those related to the generation of 3D environments based on brain activity may be suitable for both the metaverse and gaming industry, as well as those relating to the personalization of an avatar. Other applications may be more directly related to the generation of specific content either for the metaverse or the gaming industry. For instance applications related to the creation of adaptive brain-based A.I. for performance training may have direct implications for the gaming industry, whereas applications related to “neuro-architecture” and the real-time adaptive manipulation of a 3D scenario may be more relevant for the metaverse.

Some embodiments described herein are further illustrated by the following examples and detailed protocols. However, the examples are merely intended to illustrate embodiments and are not to be construed to limit the scope of the technology described herein.

### Avatar and Assets Generation

In some embodiments, a DMDT, and corresponding NFTs, may be used in videogaming applications to inform the creation of a digital avatar for gaming purposes. Brain performance indexes extracted via DARWIN, NEURO-A.I., and/or cognitive/behavioral characteristics may be used to guide the selection of features (e.g., avatar's abilities such as, but not limited to, strength, speed, stamina, executive functions), by constraining the selection of available options on the basis of DMDT features (e.g., high brain plasticity index allows for selection of higher levels of executive function and adaptation; high psychomotor reactivity allows for selection of higher levels of speed and dexterity).

In some embodiments, a neuro-avatar created via DMDT and DARWIN may be used as the backbone for multiple avatars related to multiple games, increasing inter-game accessibility and shared experience across platforms/games.

In some embodiments, BRAINPRINT and DARWIN may be used to create avatars used for non-gaming Metaverse experiences related to, but not limited to, entertainment (e.g., virtual concerts, virtual shows), content-exchange markets, IP generation events, or social media.

In some embodiments, BRAINPRINT and DARWIN may be used to create a Self-Sovereign Identity (SSI), with the aim to protect users via Decentralized Identifiers (DIDs). Information generated by BRAINPRINT and DARWIN may be coded in DIDs that can verify identity and ownership between two parties without the need to reveal detailed personal information or store the information on a centralized server.

In some embodiments, BRAINPRINT and DARWIN may be used to create content and assets related to a videogame or metaverse experience, for instance, a particular weapon or tool whose characteristics (including, but not limited to, strength, durability, speed, build quality) are constrained by features of an individual brain, cognitive and performance data. In-game/In-app features, assets and functions may be constrained by an individual's features, creating the need and opportunity to improve such features in order to unlock new assets or improve upon existing ones.

### Cognitive and Brain Enhancement

In some embodiments, a player may be forced to operate within a range of possible ability levels constrained by their DMDT. The player may be offered in-game activities aimed at training skills and features that are not optimal according to their DMDT. Completion of such activities/tasks may require a certain level of mastery of real-life skills, so that an increase in in-game ability also corresponds to enhancement of real-life abilities (e.g., in order to increase speed of an avatar, a player may be required, at least in part, to complete psychomotor speed training tasks outside the game; reaching a certain level of speed on a real-life task monitored via wearables or software, to unlock new skills in the game; the same concept may be applied to high-order cognitive skills, such as memory, abstract reasoning, or sustained attention). Skill proficiency and relative ranking within a community may be made available to other players, eliciting a competitive drive resulting in boosting of both in-game and real-life abilities.

In some embodiments, a DMDT and game-related content may be used outside the game engine, for instance as part of an app on a mobile device aimed at training an avatar on specific tasks outside the game main mechanics and narrative. This application may extend the gaming experience beyond classical gaming paradigms (e.g., beyond the life cycle of an avatar or gaming character). When a player's avatar dies in the game, content and experience may remain in a virtual “dataset” to be shared with other players.

In some embodiments, DMDT and DARWIN may be used to generate standalone applications outside a main game or metaverse, playable on a separate mobile hardware allowing for offline, off-Metaverse avatar improvement.

### Game Mechanics

In some embodiments, a game engine may use players' DMDTs to guide the selection of in-game activities more suitable for a given player's progression, creating a hierarchical table of tasks listing the optimal, personalized trajectory for in-game progression.

In some embodiments, a player may be presented with a detailed analysis of their DMDT and performance indexes, and may be provided with interactive options for skill acquisition guided by the algorithm(s) in DARWIN. The player may make an informed decision on the most suitable trajectory maximizing their gaming experience and reward. The player may become part of their in-game trajectory, acquiring knowledge on the most successful strategies for boosting their abilities in the game, and be allowed to share them with other players.

In some embodiments, the game engine may use players' DMDTs to select subpopulations of players with similar attributes to be exposed to similar in-game experiences, increasing their chances of in-game progression and mutual learning.

In some embodiments, the game engine may use players' DMDTs to select subpopulations of players with dissimilar attributes to be placed in cooperative quests or in-game activities, increasing exposure to diverse in-game attributes and gaming styles, therefore challenging players' habits, and promoting a shift in stereotypical behaviors and a player's strategy.

In some embodiments, individuals with similar or different DMDTs may be ranked and matched to maximize the change of optimal team-playing and synergy. This is applicable to various fields including, but not limited to, professional gaming experiences (e-sports), team selection (as in the case of professional sports or the creation of optimal business teams by balancing individual skills necessary to accomplish a specific task), and group therapy.

In some embodiments, the game engine may be trained to learn from a player's DMDT and behavior to improve level design and create more challenging scenarios to be played. The game may constantly adapt to progressive DMDTs in a closed-loop fashion, generating an adaptive experience, where the A.I.-controlled avatars share knowledge on the player and behave as a hive-mind. Both the A.I.-controlled avatars and the game design (including, but not limited to, scene complexity, map size, map geometry, placement of targets, number of tasks to be accomplished) may change based on an individual's DMDT and may be updated based on current performance and progression algorithms defined in DARWIN.

In some embodiments, game mechanics leveraging BRAINPRINT and DARWIN modules may be used to create a variety of game genre including, but not limited to: Sandbox games, open-world; AAA game with a single/multi player campaign; Experiential/Wellbeing games; adventure games; Cognitive training software; or Emotion recognition/monitoring videos with games.

In some embodiments, a DMDT and game-related content may be evolved based on DARWIN methods and principles including genetic algorithms, in order to create an army of clones, and let the clones evolve in different ways based on their function, and/or train them on specific abilities.

In some embodiments, content and knowledge accumulated via BRAINPRINT and DARWIN may be used to create dynamic A.I. /NPCs (non-player characters), influencing behavior via community inputs.

### In-Game Training

In some embodiments, the game engine may use a player's DMDT to create clones of the players' avatar for training purposes. By being able to visualize and analyze a clone's behavior, a player may increase awareness of their tactical, behavioral, cognitive limitations, and improve their in-game performance. Clones may be enhanced in specific abilities by a player and serve as an example of potential in-game improvement (e.g., a player may decide to boost psychomotor speed and decrease behavioral inhibition of their avatar, and test the resulting in-game behavior and performance by simulating game scenarios). Clones may be enhanced in specific abilities by the game engine and be used as enemies to provide an incremental, adaptive challenge for players, leading to more effective realistic learning (e.g., the game engine increases psychomotor speed of a series of 5 clones the player must face in a 1 vs 5 battle; the player adapts his/her in-game behavior by inferring the difference in speed). In some embodiments, clones may be used by players to create virtual “offspring” of their avatars, to be used as, at least in part, in-game companions or members of an army.

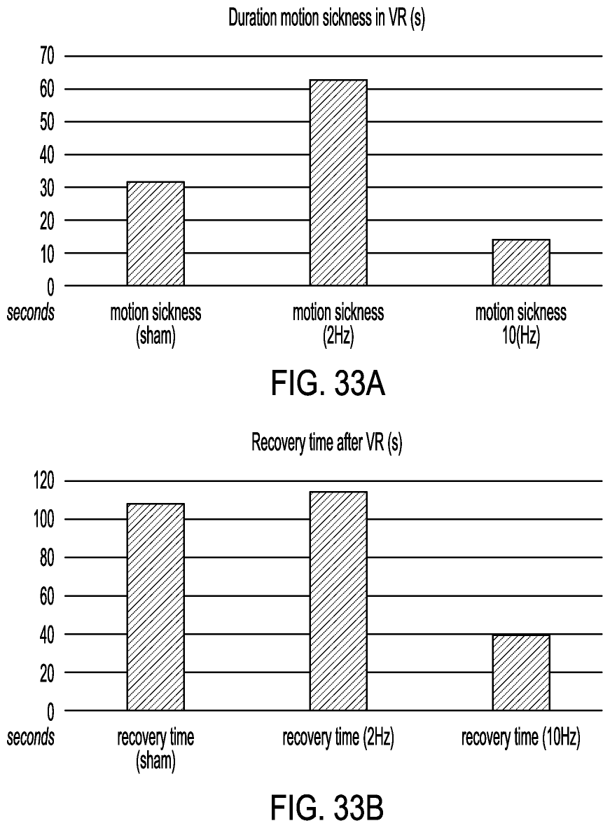

In some embodiments, clones may be equipped with a conversational A.I. agent including, but not limited to, the CLARITY agent described in connection with FIG. 33. The agent may be configured to communicate with the player using verbal and written language, suggest the best strategies for performance improvement or solve a given task, retain information on player's performance and/or update its knowledge following the principles described in association with CLARITY. The agent may have access to a player's DMDT and its longitudinal data, and may be able to spontaneously suggest modifications to a player's gaming style.

In some embodiments, clones of other players may become available to be used as A.I.-controlled enemies or partners in training settings. Information on in-game performance and behavior for a given player (not limited to, e.g., their aggressiveness, cooperative level, strategic vs impulsive style, weapon accuracy) may be summarized in an A.I. agent and made available for other players to download and be used to test their performance and improve their skills in personalized training scenarios. Players with similar DMDTs may benefit from playing against the same A.I. agent, maximizing their learning trajectory and optimizing their in-game performance more rapidly.

In some embodiments, a DMDT may be used to create and personalize a videogame training program for a given player. A player's characteristics may be used to compute the best in-game learning curve to maximize performance and accelerate learning. The DMDT may be used to identify the most relevant skills to be trained and their priority, resulting in a hierarchy of progressive steps where A.I.-controlled avatars (enemies, NPCs) are deployed based on their skills. The training process may adapt based on a player's performance and proficiency at a specific level, and novel tasks and challenges may be added by adapting the A.I. of other avatars.

In some embodiments, the DMDT of a player may be used to identify strengths and weaknesses in terms of cognitive skills, behavioral performance, in-game performance, psychological attributes, and/or brain profile. These features of an individual DMDT may be used to identify optimal game genre and specific game types that best fit a player's characteristics. The DMDT may be used to generate a hierarchy of game genre and/or game experiences the player should prioritize to maximize his in-game experience. The focus on this optimization is not game performance but game experience, including satisfaction and reward. For instance, individuals whose DMDT suggests a stronger reward response to short-term and immediate goals, may be directed towards fast-pacing games that activate the reward system; individuals with high logical reasoning skills and a propensity to process information about the future as detected via brain data recording, may be directed towards strategic games. The goals of optimizing gaming experience include, but are not limited to, maximizing brain and mental health, minimizing stress, increasing the activation of reward circuits, and/or decreasing anxiety and improving mood.

In some embodiments, analysis of an individual's DMDT may identify basic game features across multiple game genres, which may be used to create ad-hoc videogaming experiences maximizing the experience of a specific individual. The resulting videogame experience may include elements from different videogame genres, combined in a unique way. It might also include physical properties able to induce a positive mental state in the player including, but not limited to, color palettes, complexity of the environment, geometry of physical assets in game, and/or audio properties.

### Passive Brain Health Estimation and Performance Monitoring

Performance at a videogame is composed by a high-dimensionality set of inputs and behaviors that can be traced passively while an individual is playing a specific videogame. Such a complex dataset of cognitive, behavioral and physiological data can be used to track an individual's behavior within a gaming session, across gaming sessions, as well as in relation to other individual features such as those monitored and measured via DMDT and DARWIN. The possibility to passively track an individual's information with relevance for mental and brain health may provide access to information otherwise challenging or impossible to track in clinical settings; the large user base of popular videogames attracting millions of players every day may give access to an unprecedented amount of data, especially on a segment of the population that would not be accessible otherwise, e.g., healthy individuals from teenage years up to 40-50 years old. Access to such data allows to build models of healthy brain and cognitive function, create longitudinal models of a brain's ability to learn and offer insight on best strategies to accelerate learning and enhance performance, as well as build predictive models on the onset of mental-and brain-health-related conditions linked to, for instance, mood and anxiety disorders, suicidal behavior, and/or substance abuse.

For instance, patterns of visuo-motor coordination while videogaming might inform on overall visuo-motor coordination abilities of an individual, while longitudinal changes in such patterns over time might inform on the individual's ability to learn, rate of learning, meaningful variations of visuo-motor activity possibly reflecting broader changes in brain activity with relevance for brain and mental health (for instance, an overall slowing of visuomotor responsivity and coordination may be linked to a significant swing in mood and anxiety levels with potential impact on overall mental health; altered coordination might be signaling substance abuse or the onset of visuomotor coordination problems possibly linked to neuromuscular conditions).

In some embodiments, passive and active data collected on individuals while gaming may be used to build models of cognitive and behavioral patterns linked to, but not limited to, in-game performance, learning trajectories and preferential learning habits, autonomic activation and arousal state, psychomotor performance and visuo-motor / cognitive performance, metacognition, learning to learn abilities, and/or brain and mental health. Passive data include, but are not limited to, keyboard strokes (frequency, order, patterns of co-activation, speed, and keystroke pressure), mouse trajectory data, joypad data, and in-game performance data.

In some embodiments, passive data may be combined with external hardware for collection of physiological data including, but not limited to, eye-tracking data synchronized with game data and in-game events, EEG data collected via a dedicated headset (e.g., PERCEPTRON), voice data recorded during gaming sessions, galvanic skin response collected via a device placed on the fingers/hands/wrists or on the face, and/or cardiac data including heart rate and heart rate variability.

In some embodiments, passive data may be combined with active data collected via dedicated questionnaires prompted during, before and/or after a gaming session, to elicit specific behavioral, cognitive and psychological responses.

### Neuroarchitecture

In some embodiments, a DMDT may be used to inform the generation of in-game content including, but not limited to, the physics of the game and storytelling. Longitudinal DMDT acquisition may be used to update in-game content, including using real-time DMDT acquisition during gaming to adjust game features in real-time depending on players' brain state (e.g., brain plasticity index, brain emotional activation, and psychomotor vigilance). In-game features may include, but are not limited to: physics related to perception of time (e.g., game experience may accelerate or slow down on the basis of, for instance, a player's psychological status, reflecting emotional activation, drowsiness, attentional levels); physics related to space perception (e.g., game environment may shrink or expand on the basis of, for instance, a player's psychological status, reflecting emotional activation, drowsiness, attentional levels, stress level); in-game environmental factors (e.g., weather changes from sunny to a rain storm according to a player's activation level, stress level, psychosomatic reaction); story-telling, with performance indexes and brain state determining accessibility to a game section (e.g., certain areas may not be accessible until a specific level of a performance index is reached).

In some embodiments, a VR environment may be created on the basis of trait-and state-related characteristics of a DMDT. Elements of the VR environment, including structural elements (e.g., background scene, terrain, sky) and secondary elements (e.g., buildings, tools, furniture, clothes) may be generated based on specific patterns of brain activity and/or a particular DMDT configurations. An individual may be able to generate 3D objects (structural and secondary ones) based on their real-time brain activity or a specific pattern achievable through training. Generative, modular creation of virtual objects (GMVO) may happen within a regulated environment where criteria for asset generation are fixed and shared across individuals. GMVO may be integrated with existing sandbox platforms including videogames such as, but not limited to, Minecraft (available from Microsoft Corporation) or Fortnite (available from Epic Games, Inc.) videogames, where an individual may create in-game assets as part of a persistent 3D online experience.

In some embodiments, the quality of the assets may be defined based on DMDT indexes capturing features of brain activity measuring aspects including, but not limited to, the strength of brain activity (e.g., amplitude or frequency of brain signal), its variability and stability over time, localization in the brain, and/or modulation over time.

In some embodiments, an expansion of GMVO may include the creation of both physical objects, game properties and coding language generated via the B2M2C and AIG platform. Generation of code including, but not limited to, logical and conditional coding describing the interaction between players and the gaming environment, the interaction between players, and the interaction between digital assets, may be generated directly from brain data recorded via a device including, but not limited to, PERCEPTRON. Brain data may be processed via PREPARE and features may be extracted via B2M2C, creating a series of language-based commands for the generation of assets and their interaction that are transformed into code and 2D/3D assets by AIG.

In some embodiments, AIG may use principles adapted from computational geometry and procedural triangulation for the generation of 2D and 3D models based on a novel algorithm for geometrical approximation and voxel-based reconstruction of surfaces (referred to herein as “BUILD”). The inventor has created a method for generating 2D and 3D digital assets from an initial input including, but not limited to, a voice command describing an object, a text command describing an object, and a brain activity pattern analyzed via B2M2C and converted into a text command. The command, defining a single digital model, may be converted into a set of N 2D images of the model using a natural language processing agent in NOMAD-E; the user may select one of the 2D images as the reference model; the user may also select multiple images to generate an hybrid of the source images; once the reference model is defined, the BUILD algorithm may compute the linear distance of each pair of vertices of the reference image, using, for example, a spherical approximation method. The method allows to identify 3D projections from 2D images, approximating a 3D structure from a 2D image. The method may then identify the shortest distance in the image, and begin to generatively add triangles to fill the surfaces originating from the two points connected by the shortest distance. The algorithm may minimize the numbers of triangles needed to cover a given surface by minimizing the angles of each triangle. By using spherical registration (e.g., similar to spherical registration used for brain surfaces reconstruction), the method may iteratively compare the resulting 3D surface to the 2D reference, assigning error values to each triangle. The process may continue until all the triangles have been added and no broken surfaces are present. A final step may include hyper-sampling of each surface, and smoothing with spatial kernels. In an example application, an individual may think about “creating a red chair with blue pillows”; brain data collected via PERCEPTRON may be processed via PREPARE, then sent to B2M where brain signals are converted into minimas of brain activity (categories=2; objects=2; colors=2); minimas may be sent to M2C and transformed into commands (“Chair; Pillows; Red”); the input may be forwarded to AIG where the NOMAD-E natural language processing module may be used to create ten images based on “create/generate-red chair-blue-pillows”; the images may be sent back to the individual who can chose the reference image or let the NOMAD-E select one randomly; the image may then be analyzed via BUILD and the voxel-based triangulation approach; the resulting 3D model may be generated and saved as a mesh file, or immediately displayed in a live 3D environment in the case of closed-loop, real-time BUILD applications. In the proposed example, an individual may generate assets by thinking of 3D objects, modifying the surrounding digital environment; objects may then be positioned in a specific location via an interface; the objects' location can also be extracted from brain data (“a red chair close to the brick wall”). BUILD can be used for real-time world-building and for interactive world-building by two or more users combining their brain data (see example applications below for an example of hybrid asset generation).

In some embodiments, the systems and methods described herein include tools to artificially recreate an Enriched Environment via Virtual, Augmented or Mixed Reality, where a specific selection of stimuli and stimuli properties are used to build plasticity-inducing scenarios. Stimuli and their properties may be selected based, at least in part, on physical properties of objects, their interaction capability with human agents, and their effect on brain structure and function. Stimuli may be organized on the basis of their impact on plasticity, and combined to create optimal stimulation also according to individual brain properties as defined via BRAINPRINT. Additional details are provided in the Neuroplasticity section herein.

### Social Interaction and Brain-to-Brain Interaction

In some embodiments, players may exchange or trade all or a portion of their DMDT information with other players. Players may exchange a successful in-game avatar development trajectory derived from their gaming experience and trial-and-error process.

In some embodiments, players may be given the option to merge, combine or fuse all or a portion of their DMDT and related game-content (including, but not limited to, their avatar's profile(s)) with that of other players, allowing to create hybrid clones for training or content-creation purposes.

In some embodiments, players may connect in a virtual scenario via their avatars while wearing an EEG sensing device (e.g., PERCEPTRON), with their interactions being monitored in the context of EEG hyper-scanning methods.

In some embodiments, brain data collected in real-time inside the game may be combined to generate hyperparameters used to create assets and conditional rules of the game.

In some embodiments, brain data may be used as input for conditional algorithms guiding in-game performance. For instance, alignment between brain activity levels of two or more players may be required to operate an object in game, to solve a puzzle, or to obtain access to a specific area of the game. The specific features of brain activity used as input for the conditional algorithm are defined based on players' individual DMDT by looking at similarities of brain activity, thus maximizing the chance of players' brain data alignment. When alignment is not achieved, the B2M2C algorithm may suggest specific training protocols for one or more players, to align their brain response.

### Brain Health Applications

In some embodiments, a VR scenario may be created to guide a breathing protocol aimed at modulating the flow of cerebrospinal fluid (CSF) in the brain of an individual. CSF is responsible for clearance of waste products in the brain, including proteins such as amyloid-β, tau protein, alpha synuclein and TDP43 protein. Breathing offers a unique opportunity to control CSF flow given its simultaneous automatic and controllable nature, allowing for parametric manipulation of breath cycles which can have an impact on cardiac output and consequently CSF production via structures including the choroid plexus and intracranial pressure. CSF flow varies through the lifespan, with a decrease in the rate of replenishment cycles occurring during a day. This decrease leads to less newly formed CSF circulating and consequently less protein clearance. Protocols able to manipulate CSF flow will have an impact on protein clearance and other clearance processes in the brain and central nervous system, particularly relevant as countermeasures to aging, neurodegenerative disorders characterized by aberrant accumulation of proteins in the brain and altered protein clearance mechanisms. These conditions include dementia and Alzheimer's Disease in particular. A VR gamified experience may be created where audio-visuo stimuli are used to guide the breathing cycle of an individual in conjunction with a closed-loop system for wearable monitoring of the breathing cycle and adaptation of the stimuli to maintain a desired breathing output.

In some embodiments, breathing control may be achieved by the activation of the locus coeruleus, a brain structure responsible for norepinephrine release into the brain with a particular link to the hippocampal area and memory function, in particular response to novel stimuli. Activity of the locus coeruleus is also linked to controlling breathing cycles, and early accumulation of tau protein which is considered a key pathological substrate for the development of dementia and Alzheimer's disease in particular. Modulation of locus coeruleus activity may be used to boost protein clearance and tau protein removal, using a combined system including a VR based app to induce modulation of breathing cycle by controlling the pace of exhalation and inhalation. In some embodiments, a cognitive stimulation app may also used, where stimuli are presented including some repeated (known) stimuli and some novel stimuli, thus inducing a novelty effect able to activate the locus coeruleus. In some embodiments, activation of the locus coeruleus may be achieved via activation of the vagus nerve, a nerve associated with breathing cycle. Stimulation of the vagus nerve may be achieved via electrical stimulation within the ear or via an implanted electrode, at a frequency between 1 Hz and 5000 Hz, and may be synched with phases of the breathing cycle to amplify the effect of stimulation. Parameters for stimulation may be personalized using DARWIN and DMDT, determining stimulation intensity, frequency and duration on the basis of, but not limited to, electrophysiological, neuroimaging and behavioral data (for instance, breathing cycle frequency and phase) of an individual.

In some embodiments, breathing control protocols may be combined with VR-based applications simulating an avatar representing an individual positioned in different postural positions in a virtual environment. Body position in space is a modulator of cardiac and pulmonary activity, and its manipulation may be performed to induce paradoxical misperception of body position and modulate CSF dynamics and the activity of the glymphatic system.

### EXAMPLE APPLICATIONS

Some embodiments described herein are further illustrated by the following examples and detailed protocols. However, the examples are merely intended to illustrate embodiments and are not to be construed to limit the scope of the technology described herein.

Promotion of Brain Plasticity via Virtual Augmented Environment A virtual augmented environment (VAE) was created by manipulating features of the 3D environment, systematically providing the highest level of “Environmental Complexity” in order to enhance brain plasticity by stimulating neural activity in predefined patterns known to elicit synaptic modulation and an increase in brain plasticity. Manipulation was performed according to the Plasticity Maximization Algorithm (PMA). Elements of the environment were manipulated according to multiple parameters and at different levels. Parameters included:
(i) physical properties of objects including, but not limited to, color, shape, dimension, brightness, texture and texture patterns;
(ii) movement including, but not limited to, speed, direction, trajectory, acceleration, rotation angle, translation path;
(iii) sound including, but not limited to, rhythmic auditory stimulation as simple as a midi file and as complex of a multi-instrument song;
(iv) modification of (i) and (ii) including, but not limited to, changes in dimension, shape, color, speed, and sound, resulting in dynamic objects transitioning from one state to another according to parameters including, but not limited to, frequency, change rate, persistence, fusion frequency, fusion ratio; and
(v) order/sequence of features related to (i), (ii) and (iii) including, but not limited to, the presentation order of changes in physical properties of an object (e.g., first change in dimension, then shape and color combined) and presentation order of visual and audio stimuli (e.g., concurrent, sequential). The order/sequence may be defined on the basis of principles of psychophysics describing the most effective sequence and timing of visual or auditory stimuli to induce the strongest possible neural activation in brain structures responsible for processing visual and auditory stimuli. For instance, stimuli with a frequency of change higher than, on average, 40 Hz are known to not be perceived by the visual system as moving objects, resulting in a static image; therefore, complexity manipulation via shape shifting objects may only be achieved with a shifting frequency less than 40 Hz. These parameters may be personalized for each individual, given known differences in threshold for static perception of moving objects.

Parameters were manipulated and implemented at separate levels of the environment, including but not limited to:
(i) Backbone architecture including, but not limited, to the shape and size of the background 3D environment hosting the 3D experience;
(ii) Primary objects including, but not limited to, scenic elements such as buildings, architectures, body of water, vegetation, elements representing a sky if present;
(iii) Secondary objects including, but not limited to, scenic elements such as cars, inanimate objects including tables, chairs, windows, tools, weapons, clothes;
(iv) Avatar level including, but not limited to, features of a person's avatar such as body shape, size;
(v) Interactive level including, but not limited to, features describing interactions across objects in the 3D environment, such as combinations of multiple objects to create more complex patterns, fusion of multiple objects, objects with active functions controlled by the avatar (e.g., weapons, switches, control panels); and
(vi) Social level including, but not limited to, features related to A.I.-controlled non-playable characters (NPCs) interacting with the avatar, as well as avatars of other human players.

The complexity and “enrichment factor” of the virtual augmented environment was quantified by an Environmental Complexity score, measuring the presence and number of objects and related manipulated parameters present in the 3D environment at any given second of the experience. For instance, a 3D environment composed by a white cube with a red building at the center and no interactive features for the avatar to operate, constituted a complexity score of 3 (cube+building+color change); the same environment now comprising multiple buildings of various colors, a table with interactive tools for the avatar to interact with, a blue sky and the sound of a waterfall in the background, constituted a complexity score of 11; the same environment with rotating floating cubes changing size and color based on the avatar's movement, an interactive panel for the avatar to control the weather, demolish existing buildings or create novel one using a modular construction tool, constituted a complexity of score of 23; the same environment with adaptive background music changing based on the avatar's proficiency at a given activity and the possibility to drive vehicles, constituted a complexity score of 26; and the same environment with non-playable characters (NPCs) interacting with the avatar by offering quests and challenges, constituted a complexity score of 30.

Resulting scenarios may be composed by abstract or realistic environments and be personalized based on individual features captured via an individual's DMDT and estimation of brain plasticity performed using DARWIN based on brain and cognitive data. For instance, an individual with low contrast sensitivity as measured via a visual acuity test, may be exposed to an environment where complexity is defined via changes in objects' shape and audio and less on color and brightness. An individual with high tolerance to complexity, for instance a videogame player expert on realistic first-person shooter games and having high visuomotor coordination, may be exposed to high levels of complexity obtained via the presentation of abstract, shape-shifting 3D objects following a behavior not adhering to laws of physics.

In an example study, healthy volunteers were presented with 3D scenarios of increasing complexity following the criteria described above (with a complexity score from 4 to 49), presented using a VR headset. Measures of brain plasticity were collected before and after exposure to each level of complexity, via behavioral assessments in the form of cognitive adaptability tests and brain plasticity measurements according to methods included in DARWIN. Each scenario was presented for 30 minutes.

The level of plasticity significantly increased in a non-linear fashion after exposure to more complex VR scenarios. Lower complexity scenarios below a complexity score of 21 did not evoke any change in brain plasticity, with a linear increase in plasticity levels from complexity 26 to 49, as shown in FIG. 31, which illustrates modulation of brain and cognitive plasticity as a function of environmental complexity.

In this example, it was demonstrated that a virtual environment promoting brain and cognitive plasticity can be built by systematically manipulating physical properties of a 3D environment using the Plasticity Maximization Algorithm-PMA, resulting in 3D experiences that may be used as standalone plasticity-inducing interventions or integrated as part of existing 3D experiences including, but not limited to, videogames, movies and interactive documentaries.

In some embodiments, similar parameters and levels of manipulation may be used to create plasticity-inducing 2D environments, including movies and videogames displayed via media such as TV, monitors, and smartphones.

In some embodiments, Virtual Augmented Environments may be coupled with drugs acting on brain plasticity, such as ketamine and those acting on the PNN and extracellular matrix.

In some embodiments, Virtual Augmented Environments may be coupled with noninvasive brain stimulation techniques acting on brain plasticity, such as high frequency TMS and transcranial electrical stimulation targeting the PNN and interneurons' activity.

In some embodiments, Virtual Augmented Environments may include parametric distortion of the visual field as a method to retrain the visual system and induce plasticity in the brain. Adaptation to visual field changes, such as in the case of the application of prismatic lenses, may be used to alter visuo-motor perception and force remodeling of connectivity pathways in the brain, increasing levels of plasticity and enhancing the effect of add-on interventions including but not limited to cognitive training, rehabilitation and psychotherapy. The effect of prismatic lenses may be simulated via manipulation of the field of view during a VR/AR experience, in combination with visuo-motor training as in the case of point-click tasks and motion pursuit tasks.

### Reduction of Motion Sickness, Vertigo and Nausea During VR

Motion sickness, including nausea and vertigo, are commonly reported phenomena while experiencing VR using a head-mounted VR headset. Such symptoms and sensations are widely recognized as one of the most relevant obstacles for broader VR adoption, making solutions to alleviate such sensation a key unmet need for the Metaverse field and for VR applications in general. Attempts have been made by means of visual cues displayed in VR with the intent to encourage a user of the VR system to focus their attention on the center of the visual field, thus reducing motion sickness. However, these solutions produce limited effects and dramatically alter the VR experience by limiting the field of view of the user, thereby changing the VR experience as a whole. This and similar solutions only affect the VR experience from a behavioral perspective, without affecting the neural source of VR-related sensations.

Some embodiments of the system and methods described herein include a solution for reducing motion sickness during VR by means of transcranial electrical stimulation of the brain. PRINT and DARWIN were used for the identification of the optimal brain targets as well as parameters for stimulation. Results of a study conducted by systematically inducing VR-related motion-sickness in a group of healthy individuals and concurrently applying a specific form of tES, transcranial alternating current stimulation (tACS) (see STIMOLA), targeting a brain functional network defined via PRINT and DARWIN are reported below.

Participants performed multiple study visits during which subjective sensations and physiological data were measured as the participants engaged with a rollercoaster simulator displayed via a VR headset. Each study visit involved multiple simulator sessions of incremental complexity (and resulting motion sickness sensation), where subjects were asked to report the onset and end of motion-sickness sensations (measured by an investigator in seconds), as well as subsequently rate the intensity of such sensation on a visual analog scale from 1 to 10. Subjects were also asked to report after-effects of the simulation, specifically the duration of motion-sickness after the session expressed as the amount of time required to feel comfortable repeating the simulation (in minutes). During each session, tACS was delivered for the duration of the simulation. The same design was also applied to other VR applications inducing motion-sickness, including a driving simulator, a microgravity simulator, and a first-person shooter game.

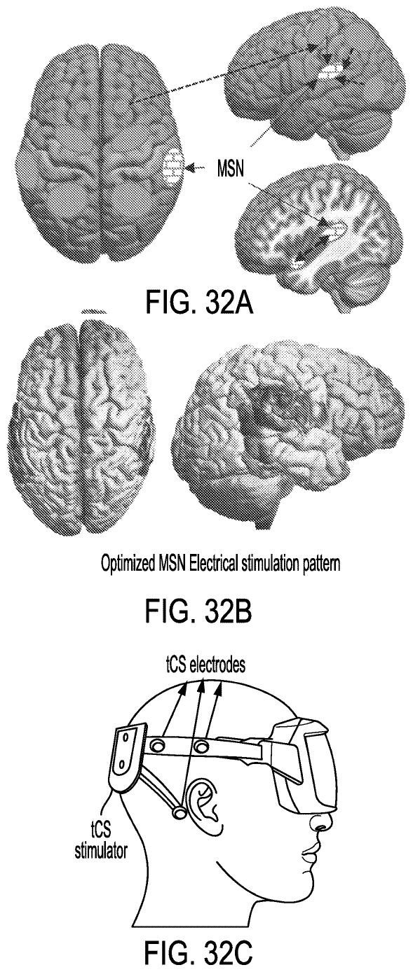

A network of brain regions was identified as a potential target for neuromodulation of motion sickness, vertigo and nausea during VR applications (Motion Sickness Network—MSN). The network included cortical regions located in both hemispheres of the brain, encompassing regions belonging to four separate systems (vestibular, auditory, motor, and visual system) as shown in FIG. 32A. Regions included, but were not limited to, portions of the insular complex, the temporoparietal junction and the supramarginal gyrus. The network of regions had a resonant frequency between 1-5 Hz, while inhibitory activity was located above 6 Hz. A pilot study using EEG was conducted on healthy participants during engagement with the rollercoaster VR app to identify the optimal range of stimulation for suppression of motion sickness, resulting in a range between 7.8-14Hz. tACS was conducted within this range. Biophysical modeling was conducted to maximize electrical stimulation of the target regions, minimizing stimulation of the rest of the brain as shown in FIG. 32B. tACS electrodes were placed underneath an elastic band holding the headset and in proximity of the right and left ear as shown in FIG. 32C.

tACS was delivered at different frequencies, including a sham (placebo) condition. The sessions were conducted over multiple days, with a different frequency each day. The frequencies were Sham (placebo), where stimulation was not delivered; ˜10 Hz, as the target frequency responsible for suppression of motion-sickness as identified by DARWIN as well as a frequency associated with inhibition of activity in the vestibular system of the human brain; ˜2 Hz, as a control condition matching the resonant frequency of the vestibular system and therefore potentially responsible for inducing motion-sickness. Stimulation was applied with a personalized montage including scalp electrode locations and intensity generated via DARWIN. Intensity was kept below 2 mA per electrode and 4 mA across the entire electrodes array.

Participants were not able to distinguish real vs placebo stimulation. Stimulation was perceived minimally at the scalp level, with no side effects reported. As shown in FIG. 33A, participants reported an average duration of motion sickness/nausea/vertigo of ˜30 seconds during sham—placebo—tACS, ˜62 seconds during 2 Hz tACS, and ˜13 seconds during 10 Hz tACS, suggesting a significant reduction of motion sickness during the VR experience due to brain stimulation at 10 Hz over the MSN. As shown in FIG. 33B, a similar effect was observed when participants reported the duration of after-effects, i.e. for how long the sensation induced by the VR experience lasted after stimulation ended: ˜108 seconds after sham tACS, ˜116 after 2 Hz tACS, and ˜39 seconds after 10 Hz tACS.

The same design was also applied to other VR applications typically inducing motion-sickness, including a driving simulator, a microgravity simulator and a first-person shooter game. Results were similar for all the tested applications, suggesting good generalizability of the observed effects for tACS over the MSN, and a wide range of applications for the proposed tCS approach. Tests when stimulation was targeted over control brain regions outside the MSN were also conducted, producing no statistical differences across sham and active tACS stimulation.

Tests were conducted to investigate the level of stability and balance during stimulation of the MST, by measuring reported levels of vestibular activation in the form of self-report perception of visual field disturbances (for instance, horizontal oscillation of the visual field causing loss of balance; sudden acceleration and rotation of the visual field). Participants reported increased levels of stability when exposed to ˜10 Hz tACS, with less propensity to fall or lose equilibrium when mechanical pressure to their body was applied. Visual field alterations were not present during stimulation with tACS at ˜10 Hz, and a sense of improved balance was observed when subjects were asked to rotate their head over the x, y, and z axis.

In this example it was shown that integrating PRINT, DARWIN and STIMOLA for Metaverse applications and VR applications in general, allow to reduce motion-sickness, nausea and vertigo during a multitude of VR applications including 3D navigation, static and directed exploration. The system and method include hardware and software solutions for the integration of the solution into existing and ad-hoc VR headsets, allowing to deliver electrical stimulation during VR. In some embodiments, the tACS protocol may be adapted based on individual differences in brain activity as measured via, for instance, EEG recording, using PRINT and DARWIN algorithms and data. Stimulation amplitude and frequency may be adapted to each individual DMDT, and further updated over time via longitudinal DMDT data. The MSN may be modified based on individual brain anatomy using individual DMDT, PRINT and DARWIN algorithms and data. In some embodiments, the hardware and software, based on PRINT, DARWIN and STIMOLA, may be adapted for other applications, including prevention of falls in individuals with neurological conditions affecting the vestibular systems. For instance elderly individuals where neurodegeneration and physical changes due to aging has been shown to increase the propensity to fall with resulting accelerated cognitive decline due to hospitalization and surgery. Applications include, but are not limited to, patients with Alzheimer's disease, dementia and Mild Cognitive Impairment.

In some embodiments, hardware and software, based on PRINT, DARWIN and STIMOLA, may be adapted for its application as a countermeasure to spaceflight-associated persistent motion sickness, altered motor and spatial orientation and vertigo symptomatology observed in astronaut and cosmonauts upon re-entry from long-duration spaceflights on the international space station. These symptoms are generated by proprioceptive force orientation (e.g., no feet on ground), horizon line visual (e.g., lack of up/sky reference, round tube effect), and loss of vestibular data from semi-circular canals (e.g., floating fluid-otolith dominance for accelerations), loss of acoustic clarity for head transfer function localization. Stimulation of the MSN may be used to recover after space missions and to accelerate mandatory physical training necessary to recover normal physiological levels.

### Time Manipulation Therapy

Mental health conditions can induce a change in the perception of time. This is thought to be mostly due to alterations in the activity of specific regions and systems of the brain, like those regulated by dopamine, as well as by the resulting shift in the cognitive perception and interpretation of time flow, resulting in gross under or over estimation of the passage of time, distance of future events and recency of past events. This has an impact on the interpretation of both internal and external events, with repercussions on, at least in part, the feeling of urgency related to planned actions and the emotional activation related to past traumatic memories. Perception of time is also involved in the ability to reach a so-called flow state, where perception of time is altered via suppression of an individual's internal clock and extreme focus on tasks and stimuli at hand.

In this example, the perception of time was manipulated as a tool for mental health conditions characterized by anxiety-, emotion- and mood-related symptoms. The Time Manipulation Therapy (TMT) is a digital therapy composed by an algorithm actively manipulating the physics of, at least in part, digital assets, models, scenes and games, by accelerating or decelerating the passage of time in applications experienced by an individual. TMS has a specific application in combination with VR and XR experiences, resulting in increased immersion and embodiment.

In some embodiments, TMT may be applied by modifying the perception of time via selective reinforcement of a different timescale. Individuals exposed to TMT may be immersed in a realistic 3D environment with (i) direct time references including, but not limited to, a visible clock, a digital display, a calendar, and (ii) indirect time references where the length of perceived events is artificially shortened or lengthened. In an example scenario, an individual may be waiting for a train at a train station where a display shows a train arriving in 7 minutes; the time-factor in the VR application may be set to 1.35%, resulting in the train arriving in 4 minutes and 55 seconds. The same principles may be applied to different timescales, from milliseconds to minute to hours to days to weeks to months to years to decades.

In some embodiments, manipulation of time may be applied to exposure to (i) external stimuli (e.g., a photo, a sound, a movie, sensory stimulation in the form of tactile stimulation) or (ii) internal stimuli (e.g., a memory of a past trauma, visual imagery of a future event, a memory of a specific individual). Duration of the stimuli may be manipulated according to the desired effect.

In some embodiments, the TMT may be applied in patients with attention-deficit/hyperactivity (ADHD) as a therapy to manipulate the perception of time and reduce the frequency of attention shifts. In some embodiments, the TMT may be applied in patients with post-traumatic stress disorder (PTSD), as a therapy to manipulate the perception of time and increase the temporal distance between the present and past traumatic events. An image or digital object may be used to trigger a traumatic memory while an individual is immersed in a VR environment and individual response is recorded; the duration of the experience may be manipulated by manipulation of physics in the VR/AR environment, resulting in altered perception of a shorter or longer exposure to a stimuli and its induced physiological and psychological response. In some embodiments, the TMT may be applied in patients with anxiety disorders and related symptomatology, as a therapy to decrease time perception and project future events and planned activities further in the future. In some embodiments, the TMT may be applied in patients with mood disorders and related symptomatology, as a therapy to accelerate time perception and decrease the persistence in a subjective perceptual negative state, further leading to increased motivation and goal-oriented actions. In some embodiments, the TMT may be applied in patients with dementia and related symptomatology, where alterations of time perception can lead to confusion and disorientation.

In some embodiments, TMT may be used to manipulate the perception of time passage during the day, with the goal of modifying biological and physiological signaling related to circadian rhythms. A goal of this application is to use the system to affect circadian rhythm and related brain and cognitive functioning including, but not limited to, sleep cycles, brain oscillatory activity and attentional levels. TMT may be applied via a variety of stimuli, including the recreation of realistic 3D environments with manipulation of time reflected in changes of weather and physics conditions including, but not limited to, light levels, dawn- or dusk-like sky representation, presence of fog, animal sounds representative of different hours of the day. In an example application, an individual exposed to accelerated daytime (1.5 hours to experience 9 hours) experienced via a Virtual Reality environment in the morning experienced a significant change in alpha activity in the brain as measured via PERCEPTRON, with a decrease in spectral power similar to levels observed during late hours of the day. Manipulation of brain activity via TMT may be used to induce sleep-related processes including, but not limited to, CSF circulation and protein clearance which are otherwise normally expressed during night hours, to enhance brain plasticity, to promote memory consolidation, to decrease anxiety levels, and to reset the circadian clock in the case of time-zone induced mismatch (jetlag). TMT may be embedded in existing digital experiences including, but not limited to, videogames.

### Adaptive-audio Stimulation for Brain State Optimization, Cognitive and Performance Enhancement

In this example, optimization of brain state, as well as cognitive and performance enhancement, may be achieved via an adaptive, closed-loop manipulation of brain activity using multisensory stimuli delivered in the form of audio, video, tactile stimulation or any combination thereof. Resulting modulation of brain activity may generate modulation of cognitive and behavioral performance, allowing for dynamic assessment of brain state and on-the-fly deployment of ad-hoc stimuli able to tune brain activity towards a desired state. In some embodiments, states may be represented by cognitive states including, but not limited to, a state of high or low attention, creativity, focus, flexibility, idea generation, memory recollection, language proficiency, abstract reasoning, and perceptual awareness. In some embodiments, behavioral states include, but are not limited to, a state of high or low visuo-motor speed, visuo-motor coordination, aerobic output, motor speed, agility, pain tolerance, and/or physical strength.

In this example, BRAINPRINT AND DARWIN were used to develop an algorithm for assessment of performance and delivery of adaptive stimuli, which was tested in healthy volunteers (Adaptive Brain State Optimization-ABSO). Analysis of brain, cognitive and behavioral data via BRAINPRINT was used to generate a DMDT including information about specific brain states related to high and low cognitive and behavioral performance. The application of DARWIN metrics was used to identify a target state and create ad-hoc auditory and visual stimuli able to drive brain activity and performance towards the target state. Data were collected using noninvasive electrophysiology measures including galvanic stress response (GSR), heart rate and heart rate variability (HR/HRV), spontaneous and evoked brain activity via mobile electroencephalography (EEG). Metrics of brain efficiency were extracted using data analysis methods from PREPARE and DARWIN OPTI-COG modules. Behavioral data indexing visuo-motor reaction time and attention levels were collected during audio-visual stimulation.

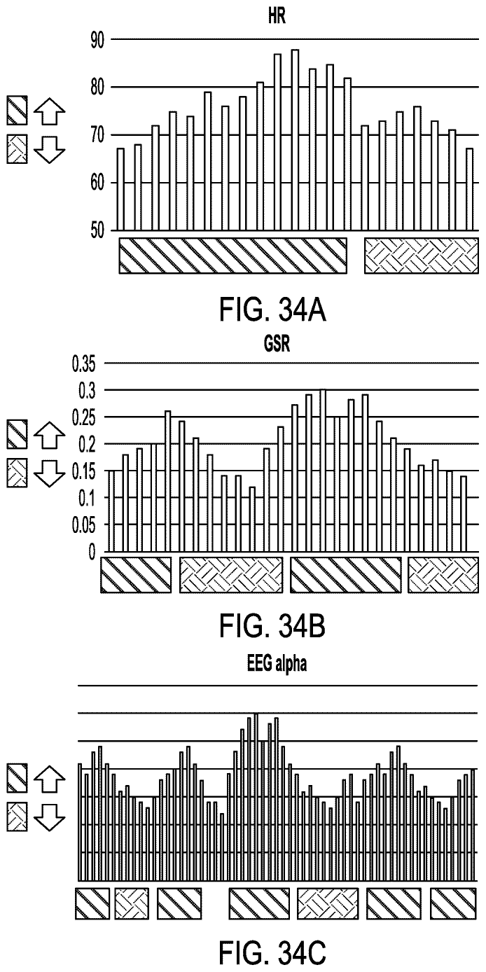

Adaptive stimulation was delivered for blocks ranging from 10 to 70 seconds, on the basis of measured changes from HR, GSR, EEG and behavioral data (state-data). Individuals were exposed to an initial baseline assessment during which HR, GRS and behavioral data were collected and used to determine a baseline brain state and corresponding activation level. Stimulation was delivered via headphones and a VR headset. Audio stimuli were selected based on their rate of “activation” determined via their beat per minute (BPM) ratio. A set of audio stimuli with an equal representation for stimuli inducing an increase or a decrease in brain and behavioral activation was identified, and stimuli were initially presented randomly in order to test their impact on brain state. Subsequently, stimuli were delivered based on online analysis of state-data, verifying the feasibility of steering brain state in real-time based on audio-guided state transition. As shown in FIG. 34A-34C, audio-guided transition was successful in modulating brain activity and physiological state as a function of BPM, with a directional shift mimicking the changes in stimuli activation level. Audio stimuli were then deconstructed to their nuclear elements including, but not limited to, base rhythm, dominant frequency, volume shifts per second, number of progressive scales. Nuclear elements were associated to levels of induced activation or deactivation, and recomposed to represent ad-hoc stimuli maximizing the induction of the target state. The new stimuli were presented to subjects using the same adaptive scheme described above, with an improvement in the rate of response to the stimuli (as measured via GSR/HR/EEG data) as compared to the original audio stimuli. In a third condition, audio stimuli were also matched with video stimuli either matching or mismatching the BPM or activation level of the audio stimuli, which were presented via a VR headset. Video stimuli were composed of moving objects of increased complexity, following the principles described in the Virtual Augmented Environment (VAE) section of the SYNAPSE section of the systems and methods. Matching stimuli were more effective in inducing a state change corresponding to the desired state direction (increased or decreased activation).

FIGS. 34A-C show responses to ABSO. Modulation of physiological and brain state as a response of activating and deactivating patterns of auditory stimuli. HR (FIG. 34A), GSR (FIG. 34B) and EEG (FIG. 34C) data were processed using PREPARE.

In this example it was demonstrated that ABSO is able to induce changes in brain state, cognitive and behavioral performance, via adaptive analysis and deployment of biosignals and behavior. Characteristics of audio, visual and other stimuli are modifiable on the basis of desired target state. The ABSO was delivered via dedicated hardware (e.g., PERCEPTRON), but its algorithms and operating principles are applicable to other mediums, such as monitors, headphones and speakers.

In some embodiments, ABSO may be used for performance enhancement related to cognitive training and cognitive rehabilitation interventions. In some embodiments, ABSO may be used to induce brain states of heightened cortical plasticity, with the goal of accelerating learning, encoding and retention of novel information. In some embodiments, ABSO may be used to induce a specific brain state previously associated with a visuo-audio pattern through conditioning. In some embodiments, ABSO may be used for performance enhancement during training applications, as a tool to boost physical performance and mental focus. In some embodiments, ABSO may be used to induce brain states related to recollection of memories and past traumas, with applications in the field of psychotherapy.

In some embodiments, music may be decomposed to the level of single notes and patterns of notes, and their association with particularly evoked brain states may be used to compose songs able to trigger a specific response in the brain in terms of spatial and temporal activation of brain regions, networks, and/or circuits. First, a test was conducted to quantify the response to specific audio stimuli while recording brain activity using, for instance, EEG. Brain activity patterns associated with specific audio stimuli were identified. The desired brain states were also identified and their corresponding audio stimuli. An audio file was then created by combining audio stimuli able to induce the desired brain state.

In some embodiments, ABSO may be embedded in existing media, including but not limited to songs, movies, podcasts, videogames and metaverse applications, to optimize cognitive state, performance and overall user experience.

### Generative Environment Creation

In this example, a tool for adaptive, generative creation of 2D and 3D environments was created and configured to extract features from brain data and/or DMDT information and generate in-game assets and properties including, but not limited to, the physics of the environment, weather condition, spatial resolution of in-game assets, avatars' characteristics. Generative creation of game properties may happen from existing data collected from an individual (DMDT, DARWIN), or real time brain data collected via a device (PERCEPTRON), and processed via PREPARE for data cleaning, B2M2C for extraction of features and logical commands, and AIG for the generation of corresponding in-game assets and game mechanics. A particular instance of B2M2C and AIG was created to enable the combination of brain data and collaborative generation of in-game assets and properties. The instance, called FUSE, performs two main tasks: (i) run a similarity analysis between the commands generated via B2M2C of two (or more) players, identifying similar outputs that are used to generate the main structure of the hybrid game environment built from players' brain activity; (ii) identify differences, which are used to generate player's specific assets and mechanics that are going to be introduced into the game upon agreement between the players. For instance, in an example application, players were asked to provide data to be included in their DMDT, including their resting-state EEG data, cognitive profile including memory, attention, abstract reasoning scores, personality information as quantified via a big-five personality trait questionnaire, their individual structural and functional MRI data. Data from their DMDT were used to extract information to build the core features of the gaming environment and mechanics, using FUSE to look for similarities. The players were asked the fusion ratio between their DMDTs, which was set at 50%-50%, resulting in a game design reflecting both players' DMDT in equal parts. The resulting gaming environment, which was set as a 3D world by the players, was composed by a large landscape [player 1-2], with mountains [player 1] and a big forest at the center [player 2], a lake [player 1-2], a village [player 2], no animals and 3 NPCs [player 1-2]. The weather was cloudy [player 1], with occasional storms [players 2]. The game mechanics included a quest involving retrieving an object [player 1], with enemies to be defeated and a monetary reward in case of success [player 1-2].

In a second application, players were asked to wear an EEG headset (e.g., PERCEPTRON) and were asked to perform specific cognitive tasks involving mental imagery, as well as to perform unconstrained mind wandering. The game design and mechanics were updated every ten minutes on the basis of both players' brain activity, with changes including, but not limited to, weather conditions (sunny vs cloudy; no storms), number of animal NPCs wandering the map, level of natural resources available to the players, number of hints and suggestions available to the players to solve the quest.

The application was used as a demo to test the interaction between players during a generative, collaborative world-building session. Further testing involved additional features available for DMDT-based and real-time manipulation. In some embodiments, players may be asked to solve minigames together and alignment of their gaming inputs constitutes a requirement to solve a task together; for instance, in a scenario, three wizards were asked to cast a spell to open a door and only a specific ratio of their brain activity was able to unlock the spell. In some embodiments, players may be able to change map size and alter the geography of the gaming world, by creating, for instance, mountains, rivers, grow forests, flatten entire areas, based on their real-time brain activity via B2M2C and AIG inputs. In some embodiments, the spatial resolution of in-game assets may be manipulated based on brain activity and DMDT information, with the on-screen resolution of objects in game shifting from low resolution (low-poly, equal to 430*250) to high resolution (1024*980) to 4K resolution, thus generating more or less detailed objects and overall experiences on the basis of brain data and DMDT. This specific application is also used to create, but not limited to, conditional sub-routines where information to solve a given minigame or a puzzle or a quest are accessible (e.g., visible) depending on brain data and its resulting quality. In some embodiments, players may be asked to train a particular ability in order to change their brain activity and match the requirement for generation of specific assets or to solve a specific task, minigame or quest.

The application described in this example has multiple applications, given the generative nature of the AIG algorithm, the complexity of individual brain data and DMDT, the exponential complexity resulting from the combination of more than one brain activity dataset or DMDT, and the flexibility of AIG in generating game elements including, but not limited to, physical in-game assets, game mechanics, game physics.

In some embodiments, FUSE may be used to generate a collaborative gaming environment with the goal of improving team dynamics and communication.

In some embodiments, FUSE may be used as part of a therapeutic process, where an individual may create a 2D or 3D environment based on their brain data and DMDT, to be shared with other individuals as a tool to concretely display one's internal psychological and mental state. In some embodiments, an individual may invite friends, other players, or mental health professionals, to visit their world and share their current experience. Improvements in mental, psychological and brain health, may automatically reflect in the quality of the generated environment, allowing to monitor progress in a non-verbal manner, which might be helpful with individuals with limited communication skills. In some embodiments, an individual's environment might benefit from inputs from other individuals (including, but not limited to, a therapist, caregiver), resulting in observable changes.

In some embodiments, FUSE may be used for game creation purposes in commercially available sandbox games, significantly expanding the limits of available in-game assets and game mechanics.

1-202. (canceled)

203. A method of creating a digital replica of a brain state of a person, the method comprising:
receiving information associated with a brain of the person, the information including structural brain information and functional brain information, the functional brain information including electrophysiological data recorded from the person;
processing the received information to determine a plurality of brain metrics that characterize a brain state of the person; and
creating a digital replica of the brain state of the person based, at least in part, on the plurality of brain metrics determined from received information.

204. The method of claim 203, further comprising:
designing a brain-inspired artificial intelligence system based on the digital replica of the brain state of the person.

205. The method of claim 204, further comprising:
updating the digital replica of the brain state of the person based, at least in part, on an updated plurality of brain metrics determined from updated information associated with the brain of the person; and
updating the brain-inspired artificial intelligence system based on the updated digital replica of the brain state of the person.

206. The method of claim 205, wherein the updated information associated with the brain of the person includes data sensed by a wearable device worn by the person.

207. The method of claim 204, wherein the brain-inspired artificial intelligence system is designed based on oscillatory generators reflecting the brain state and a brain architecture of the person.

208. The method of claim 204, wherein the brain-inspired artificial intelligence system is designed based on macroscale brain architecture data of the brain of the person.

209. The method of claim 204, wherein the brain-inspired artificial intelligence system is designed based on a multilayer hierarchical inference architecture including two or more layers representing convergent, divergent, and combined general cognition.

210. The method of claim 204, further comprising:
decomposing the digital replica of the brain state of the person into basic units of information; and
using the brain-inspired artificial intelligence system to generate code to control hardware and/or software using the basic units of information.

211. The method of claim 204, wherein the brain-inspired artificial intelligence system is designed based on an oscillatory generator having networks with activity calculated via neural mass models composed by different classes of neurons with different functions.

212. The method of claim 204, wherein the brain-inspired artificial intelligence system is designed based on an oscillatory generator having a fixed life-cycle that defines a metabolic budget for the brain-inspired artificial intelligence system.

213. The method of claim 204, wherein the brain-inspired artificial intelligence system is configured to be used as a conversational agent in a therapeutic setting.

214. The method of claim 203, wherein the received information includes data sensed by a wearable device worn by the person or via at least one neuroimaging method.

215. The method of claim 203, further comprising:
creating first videogame or metaverse content based, at least in part, on the digital replica of the brain state of the person.

216. The method of claim 215, further comprising:
receiving updated information from a network connected device;
updating the digital replica of the brain state of the person based on the received updated information; and
creating second videogame or metaverse content based, at least in part, on the updated digital replica of the brain state of the person.

217. The method of claim 215, wherein the first videogame or metaverse content comprises a multilayer avatar, in-game mechanics, equipment in a videogame, artificial intelligence used to control one or more non-playing characters in a videogame, or at least one in-game progression sequence or storyline.

218. The method of claim 203, wherein
the received information is from multiple sources, and
creating a digital replica of the brain state of the person comprises harmonizing, using an algorithm, the received information from multiple sources.

219. The method of claim 203, further comprising:
receiving additional information sensed by a wearable device or a brain activity recording device; and
updating the digital replica of the brain state of the person based, at least in part on the additional information.

220. The method of claim 203, wherein
the received information is formalized as a network, and
processing the received information to determine a plurality of brain metrics that characterize a brain state of the person comprises extracting characteristics of the network using graph theory metrics and network control theory metrics.
